(19) United States
(12) Patent Application Publication
(10) Pub. No.: US 2024/0398854 A1
(43) Pub. Date: Dec. 5, 2024

(54) OPHTHALMIC FORMULATIONS AND METHODS FOR THEIR USE

(71) Applicant: PANASEEA, LLC, Midlothian, VA (US)

(72) Inventors: Byron S. LADD, Midlothian, VA (US); Christopher K. CURRIN, Manakin Sabot, VA (US); Stephen DRUMMOND, Midlothian, VA (US)

(73) Assignee: PANASEEA, LLC, Midlothian, VA (US)

(21) Appl. No.: 18/205,844

(22) Filed: Jun. 5, 2023

Related U.S. Application Data
(63) Continuation-in-part of application No. 15/868,357, filed on Jan. 11, 2018, now abandoned.

(51) Int. Cl. A61K 31/79; A61K 9/00; A61K 9/06; A61K 47/34; A61P 27/02; A61P 31/02

(52) U.S. Cl. CPC A61K 31/79; A61K 9/0048; A61K 9/06; A61K 47/34; A61P 27/02; A61P 31/02

(57) ABSTRACT

The present invention comprises buffered ophthalmic formulations and methods for antiseptically treating or pretreating ocular surfaces for invasive surgical or treatment procedures. The buffered compositions of the present invention comprise antiseptic agents within an aqueous gel or semi-gel formulation, to provide enhanced methods of treating ocular surfaces prior to surgical or other invasive procedures. Buffered ophthalmic formulations provide additional benefits to standard non-buffered preparations including decreased toxicity to the ocular surfaces, which improves post-procedural discomfort and vision.

### TECHNICAL FIELD

This invention is related to buffered ophthalmic formulations and methods for their use in treatment and pretreatment procedures.

### BACKGROUND OF THE INVENTION

Medical and surgical procedures often involve treating an anatomic surface to achieve antiseptic and anesthetic qualities, especially as a pre-treatment for an invasive procedure on or through that anatomic surface. To achieve appropriate antisepsis, antiseptic agents must be administered in forms that are biologically and chemically tolerated by the anatomic surface and preferably not absorbed by the anatomic surface so as to remain topical and kill bacteria.

The field of ophthalmology is one area of medicine and surgery that often requires treating an anatomic surface (such as the ocular surface, cornea and ocular adnexa) as part of a procedure. Over the past decade, there has been a marked increase in the number of intravitreal injections (IVI) performed for the treatment of retinal pathology. This change has resulted from the rapid adoption of intravitreal agents including anti-vascular endothelial growth factors (vEGF) and steroids for the treatment of diabetic retinopathy, retinal vascular occlusions, and wet, age-related macular degeneration (AMD). Intravitreal injections entail inserting a needle through the sclera into the posterior segment of the eye.

IVI is not without potential complications. These include endophthalmitis, a purulent infection of the intraocular fluids (vitreous and aqueous). When used, the technical maneuvers of a subconjunctival injection can often require awkward positioning and risks needle contact with potentially unsterile lids and lashes, which may then contaminate the subconjunctival space. The rate of endophthalmitis secondary to IVI has been reported to be from 0.03% to 0.8%. To date, the only proven steps to reduce the rate of endophthalmitis are using a lid speculum and washing the eye pre-operatively with a 5% Povidone-Iodine solution.

Improvements in ocular preparation and the IVI technique are desired.

The antiseptic surface preparations presently used for IVI techniques are limited in their effectiveness in part because most of the surface preparation applied is lost due to run-off due to the contour of the eye and eyelids, thereby limiting the beneficial amount of contact time between agents within the antiseptic surface preparation and the ocular surfaces.

While the use of gel and semi-gel based compositions in response to this limitation is disclosed by the inventor in the WO 2007/025142 and by Alam et al. in U.S. Pat. No. 8,759,401, to date, the benefits of ocular surface preparation have only been utilized with analgesic agents, and no gelatinous form of antiseptic is available for preparation of the ocular surface.

Conventional antiseptic surface preparations for IVI techniques are also limited in their effectiveness as they are toxic to the cornea and irritate the eye (MacRae SM, Brown B, Edelhauser H. Am J Ophth, 97(2); 221-232), which does not favor liberal and prolonged exposure of these preparations to the eye. The buffered compositions of the present invention address this limitation and provide additional improvements for patient outcomes.

### SUMMARY

An aspect of this invention is the discovery of antiseptic surface preparations which produce less pain and surface irritation than conventional antiseptic surface preparations and remain effective. Another aspect of this invention is the discovery of effective antiseptic surface preparations with reduced toxicity to the cornea when applied to the eye. An added benefit of reduced corneal toxicity is less perioperative visual decline following the procedure. Patients receiving IVI typically have reduced vision and further impairment following the procedure may limit their ability to meet the visual requirements to safely drive. Thus, with current methods of preparation, many patients are driving home after their procedures with visual acuity below what is required to drive. The present invention comprises buffered antiseptic surface preparations and methods for treating an anatomic surface of a body to achieve effective antisepsis thereon using these buffered compositions. In one embodiment the compositions of the present invention comprise antiseptic agents in a buffered viscous gel or semi-gel formulation base that, in use, remain in contact with the anatomic surface so that the selected area is rendered sufficiently antiseptic with reduced irritation to the eye. An aspect of the present invention comprises compositions and methods to be used in preparation for intravitreal injections. Another aspect of the invention is the discovery that it is necessary to separate the buffered antiseptic surface preparation from anesthetic agents to provide shelf stability. Betadine compositions mixed with the analgesic lidocaine revealed a decay in the antiseptic properties to the extent of inactivity by 2 weeks. Thus, indicating a very limited shelf-life for such compounds. In addition, as discussed above, with the improvement in the art of IVI procedures over time, the belief that subconjunctival injections of anesthetics are necessary for patient comfort has been widely abandoned. Most procedures are now performed utilizing only a few drops of topical anesthesia (e.g. lidocaine and tetracaine).

The present invention also comprises methods for making and using the buffered antiseptic surface preparations of this invention. Methods for making the buffered compositions of this invention may comprise mixing the component antiseptic agent(s), buffering agent(s) and optionally gel or semi-gel formulation(s) together in effective amounts to provide a composition that has a pH in the range of 6.0 to 8.5, preferably a near neutral pH. Where an optional gel or semi-gel is used in the buffered antiseptic surface preparation, the composition is sufficiently viscous such that the composition is retained on the desired anatomic surface but does not tightly adhere to that surface so that it may easily be removed by irrigation or other simple mechanical cleansing procedures.

Procedures in which methods using the compositions of the present invention may be employed include, but are not limited to, ophthalmic procedures like intravitreal injections, anterior chamber paracentesis, retinal cryopexy, cataract surgery, iridotomy, trabeculotomy, trabeculoplasty, glaucoma surgery, ophthalmic implant surgery, and pars plana vitrectomy.

Other fields of medicine may utilize the compositions and methods of the present invention to achieve antiseptic effects in situations such as, but not limited to, the repair of cutaneous lacerations, procedures or treatments of mucous membrane-lined surfaces, dental procedures and treatments, and myringotomies. Additionally, surgical procedures requiring skin incision could benefit from a buffered antiseptic that would cause less skin irritation.

Also provided by this invention are kits that include a container with a buffered antiseptic surface preparation, with or without a viscous gel or semi-gel and a separate container with a composition comprising an anesthetic, optionally buffered. Preferably, the two compositions are in proportions that facilitate their combination before use and provide a pH in the range of 6.0 to 8.5 for the combined compositions, preferably a near neutral pH.

### DETAILED DESCRIPTION

The present invention may be understood more readily by reference to the following detailed description of the compositions and methods of the invention and the examples included herein. However, before the compositions and methods of the present invention are disclosed and described, it is to be understood that this invention is not limited to the exemplary embodiments described within this disclosure, and the numerous modifications and variations therein that will be apparent to those skilled in the art remain within the scope of the invention disclosed herein. It is also to be understood that the terminology used herein is for the purpose of describing specific embodiments only and is not intended to be limiting.

Unless otherwise noted, the terms used herein are to be understood according to conventional usage by those of ordinary skill in the relevant art. In addition to the definitions of terms provided below, it is to be understood that as used in the specification and in the claims, “a” or “an” can mean one or more, depending upon the context in which it is used.

As used herein, the terms “antiseptic agents” and “antiseptics,” which terms may be used interchangeably herein, are substances which may be used to reduce microbial levels and are biologically compatible enough to be applied to a particular anatomic surface. Preferably, the substance does not cause substantial irritation, inflammation, dysfunctional or other undesired reactions on or within the anatomic surface or adjacent tissues or organs in an unbuffered state. Antiseptic agents used in compositions according to the present invention may be microbicidal (bacteriocidal, fungicidal, and/or viricidal) in their actions, and are intended to provide a reduction in the ambient flora in the anatomic surface onto which they are administered. “Antiseptic agents” and “antiseptics” referred to herein are sterile.

As used herein, the term “sterile” means free from bacteria or other living microorganisms.

As used herein the term “shelf stable” means able to survive long periods of storage at ambient room temperature of from 68 to 77 degrees Fahrenheit without contents therein separating, precipitating, reacting, degrading or oxidizing.

As further used herein, the terms “anesthetic agents” and “anesthetics,” which terms may be used interchangeably herein, are substances which may be used to induce anesthesia, or reversibly depress neuronal function, producing total or partial loss of pain sensation when administered to an anatomic surface or tissue.

As further used herein, the terms “gel or semi-gel formulation base,” “gel,” and “semi-gel,” which terms may be used interchangeably herein, are viscous aqueous substances which may be used to deliver antiseptic agents and other agents to an anatomic surface in compositions and methods according to the present invention.

As used herein the term “tonicity” refers to the relative concentration of solutes dissolved in solution which determine the direction and extent of diffusion. Water always moves to areas of higher tonicity. To reduce irritation, certain solutions may be formulated to be isotonic so as to have the same osmotic pressure as the tissues of the anatomic surface (e.g. ocular tissues). An equilibrium is maintained between the such tissues and the water in the solution. Certain solutions may be hypotonic such that water enters the tissues of the anatomic surface (e.g. cornea) from the solution. If the solution is hypertonic. the opposite occurs.

Ocular discomfort from application of topical antiseptics and anesthetics results from the acidic pH and hypertonicity. The discomfort associated with application of the buffered composition is secondary to hypertonicity but does not cause damage to the corneal epithelium due to acidity. Testing revealed that discomfort from acidic toxicity lasts hours longer than the short discomfort associated with buffered solutions. Acidic toxicity is also additive when multiple drops are applied.

As used herein, the term “osmolarity” is the concentration of osmotically active components in solution, which may be quantitatively expressed in osmoles of solute per liter of solution. Osmolarity may influence clinical performance of certain embodiments and it may be desirable to provide solutions with hypo-or hyperosmolarity levels of osmotically active components.

The present invention comprises compositions and methods for medical and surgical procedures or treatments involving an anatomic surface of a body. An anatomic surface of a body may include an ocular surface, a mucous membrane surface, a dermal surface, a visceral surface, or any combination thereof on or within a mammalian body.

To minimize irritation of the anatomic surface, the pH of the buffered antiseptic surface preparation should be between 6.0 and 8.5 and preferably approaches that of fluids within the anatomic surface to be treated. For example, the pH value of the liquid layer which bathes the cornea ranges from 7.0 to 8.5. To maximize ocular comfort, the pH of the buffered antiseptic surface preparation for the eye should be near neutral.

Buffering agents can be acids or bases. Buffering agents such as citrates, phosphates, borates and trimethamines can be used to stabilize the pH level of the antiseptic surface preparations to preferably provide a pH between 7.0 and 7.4. Specific examples of suitable buffering agents include boric acid, sodium hydroxide, sodium borate, sodium chloride, sodium acetate, sodium carbonate, sodium bicarbonate, sodium bisulfite, trisodium citrate, potassium tetroxalate dihydrate (KH₃(C₂O₄)₂.2H₂O), phosphate buffered saline (PBS) or borate buffered saline (BBS), at three osmolality concentrations: 270, 310 and 414 mOsmol/kg.

The compositions of the present invention comprise combinations of antiseptic agents with buffering agents and optionally an aqueous gel or semi-gel formulation base that is at a pH in the range of 6.0 to 8.5 and retained on an anatomic surface but may also be removed when desired, for example by wiping or washing with irrigation or other fluids. The present invention further comprises methods of using compositions of the present invention for medical or surgical treatment or pretreatment to achieve antisepsis on one or more desired anatomic surfaces in or on a body.

Methods for making compositions of the present invention may comprise admixing one or more antiseptic agents with one or more buffering agents to achieve the desired pH without loss of the antiseptic properties of the antiseptic agents. Where an optional aqueous gel or semi-gel formulation base is used, it is used in amounts effective to achieve the desired viscosity. Where storage stability is not an issue, other components such as anesthetics (e.g. lidocaine, tetracaine, narcaine, mepivicaine, proparacaine, and bupivacaine) and other medicaments can be mixed therein as part of the composition of the present invention, provided the desired pH is retained.

The various embodiments according to the present invention include a complete composition in a single sterile container as well as a kit of two or more sterile containers, each containing one or more components of the composition of the present invention to be combined prior to use. Each of the antiseptic, buffering agent and optional aqueous gel or semi-gel formulation can be in separate containers or they can be combined in one container. In the kits of this invention, other components such as anesthetics and other medicaments should be stored in a separate container. Separation of the buffered antiseptic surface preparation and anesthetic prior to use is preferred to extend their shelf life. Ophthalmic formulations of the invention are improvements over existing formulations and solutions due to their buffered pH. Early testing of compounds containing povidone-iodine with lidocaine revealed, not previously known to those in the arts, that lidocaine destabilized the antiseptic properties.

Though not wishing to be bound by any particular theory, it is believed that the improved comfort provided by the buffered pH of the surface preparations of the present invention, allows for prolonged exposure of the eye to the antiseptic agents and any other medicaments present in the composition. Therefore, a much higher percentage of the dose of the antiseptic agents and any other medicaments (anesthetic agents) is maintained on the eye than with conventional ophthalmic formulations and solutions. Such exposure is further enhanced by where an optional gel or semi-gel is used to increase the viscosity of the composition and therefore improves contact time.

The compositions of the present invention allow additional time for desired amounts of pharmacologically active antiseptic to be applied such that these agents may slowly spread over the ocular surface and adnexa, including cornea, conjunctiva, eyelids and eyelashes. In addition, the kits of the present invention offer the option of combining other agents (e.g. anesthetic agents) with the antiseptic, providing a more efficient one-step delivery rather than the individual administration of these agents.

The formulation of the invention provides a safe means for time release of antiseptic agents (and optionally other agents such as anesthetic agents) into the eye. The release rate depends on the viscosity of the formulation base, i.e., higher viscosity results in slower release. However, higher viscosity results improved contact time by limiting run-off which is an inherent problem of using a liquid to coat a spherical surface. It is desirable to provide a contact time of about 15 seconds to maximize the effectiveness of the antiseptic agents.

An aspect of the invention relates to the use of gels and semi-gels which provide formulations having relatively low viscosity and correspondingly a more rapid release profile. In one embodiment of the present invention, the gel or semi-gel formulation base has a viscosity in the range of about 10,000 cps to about 50,000 cps. at about 25° C. based on Brookfield (LV) analysis, from about 10,000 cps to about 40,000 cps, 10,000 to about 30,000 cps, 20,000 cps to about 50,000 cps, from about 20,000 cps to about 40,000 cps, from about 30,000 cps to about 50,000 cps, from about 30,000 cps to about 40,000 cps, or from about 40,000 cps to about 50,000 cps, and all ranges therein between.

The formulation of compositions according to the present invention may be placed in any desired dispensing container or delivery device suitable for application to the skin, gums or eye. The present invention includes a buffered antiseptic surface preparation within a dispensing container or delivery device suitable for an ophthalmic formulation. The dispensing container or delivery device may be an ophthalmic delivery system, such as a sterile ophthalmic tube, e.g., a conventional 3.5 g tube having an ophthalmic tip and containing the ophthalmic formulation of the invention, or a sterile single use container containing 0.01-10.0 g or more of the formulation.

In various other embodiments of the present invention, the dispensing container or delivery device may be a pre-loaded syringe, packaged saturated swab, a bottle, a vial, or other container or delivery device. In yet other embodiments according to the present invention the antiseptic agent may be provided to the consumer in separate or multi-chambered containers or delivery devices for mixture of other components at the time of use.

An embodiment of a composition of the present invention comprises an effective amount of an antiseptic agent, including but not limited to, povidone-iodine, benzalkonium chloride, chlorohexidine and chlorobutanol admixed with a buffering composition to provide a pH in the range of 6.0 to 8.5, including the following pH values: 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4 and 8.5.

Optionally, a viscous aqueous gel or semi-gel formulation can be included in the formulation. In another embodiment, a kit comprises a container with an effective amount of an antiseptic agent, including but not limited to, povidone-iodine, benzalkonium chloride, and chlorobutanol admixed with a buffering composition to provide a pH in the range of 6.0 to 8.5, optionally with a viscous aqueous gel or semi-gel formulation and a second container with an effective amount of an anesthetic agent, including but not limited to, lidocaine, tetracaine, proparacaine, and bupivacaine optionally admixed with a buffering composition and further optionally admixed with a viscous aqueous gel or semi-gel formulation.

The percentages and dose ratios contemplated for the antiseptic of the composition of the present invention, such as povidone-iodine, benzalkonium chloride and chlorobutanol, include an effective concentration range of about 0.01% to about 20% by weight; including about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1.0%, about 2.0%, about 3.0%, about 4.0%, about 5.0% about, 6.0%, about 7.0%, about 8.0%, about 9.0%, about 10.0%, about 11.0%, about 12.0%, about 13.0%, about 14.0%, about 15.0%, about 16.0%, about 17.0%, about 18.0%, about 19.0% and about 20.0% by weight, where “about” is up to 10% of the value reported.

The percentages and dose ratios contemplated for the gel or semi-gel, when used in a composition of the present invention, such as hydroxymethylcellulose, methylcellulose, fall within a concentration range of about 0.1% to about 15% by weight; which includes about 0.1%, about 0.2%, about 0.3%, about 0.4%, about 0.5%, about 0.6%, about 0.7%, about 0.8%, about 0.9%, about 1.0%, about 2.0%, about 3.0%, about 4.0%, about 5.0% about, 6.0%, about 7.0%, about 8.0%, about 9.0%, about 10.0%, about 11.0%, about 12.0%, about 13.0%, about 14.0% and about 15.0% by weight, where “about” is up to 10% of the value reported.

The percentages and dose ratios contemplated for the anesthetic, when used in the composition of the present invention, such as lidocaine, tetracaine, narcaine, mepivicaine, proparacaine, and bupivacaine, include an effective concentration range of about 0.01% to about 35% by weight; which includes about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1.0%, about 2.0%, about 3.0%, about 4.0%, about 5.0% about, 6.0%, about 7.0%, about 8.0%, about 9.0%, about 10.0%, about 11.0%, about 12.0%, about 13.0%, about 14.0%, about 15.0%, about 16.0%, about 17.0%, about 18.0%, about 19.0% about 20.0%, about 21.0%, about 22.0%, about 23.0%, about 24.0%, about 25.0%, about 26.0%, about 27.0%, about 28.0%, about 29.0%, about 30.0%, about 31.0%, about 32.0%, about 33.0%, about 34.0% and about 35.0% by weight, where “about” is up to 10% of the value reported.

In the kits of the present invention, one or more antibiotics may also be present in a separate container for combination with the components of the buffered surface preparation of the present invention to provide additional bacteriostatic, fungistatic, and/or viristatic effects. The pH of the final formation upon combination of the kit components fall within the range of 6.0 to 8.5, and is preferably near a neutral pH. Suitable antibiotics may include, but are not limited to: polymyxin B sulfate (from about 1,000/to about 100,000 units/gm) neomycin sulfate (from about 0.5-to about 25 mg/gm); gramicidin (from about 0.01 to about 5.0 mg/gm), zinc bacitracin (from about 100 to about 5000 units/gm), gentamicin (from about 0.01 to about 5%); chloramphenicol (from about 0.01-to about 5%);), tobramycin (from about 0.01 to about 5%);), erythromycin, (from about 0.5 to about 25 mg/gm), and tetracycline HCl (from about 0.01 to about 25%).

The optional gels and semi-gels used in the buffered surface preparations of the present invention may comprise cellulose and its derivatives, aqueous cross-linked acrylic polymers, polyacrylic acid, pluronic polyol polymers, poloxamer, other polyols, carboxy vinyl polymers, other carbomers, or other biologically inert materials that will not contribute to irritation of the cornea or other ophthalmic structure. Suitable gels or semi-gels for use in the buffered surface preparations of the present invention are commercially available.

In a particular aspect of this invention, the optional gels and semi-gels used in the buffered surface preparations of the present invention are based on cellulose. The optional gels and semi-gels based on cellulose may comprise any substance derived from cellulose that forms an aqueous gel or semi-gel at a desired viscosity, i.e., is derived from cellulose, is soluble in water and forms a gel or semi-gel. Such cellulose derivatives are well known, as are their properties, and are described, e.g., in the U.S. Pharmacopeia 2005 (United States Pharmacopeial Convention, Inc., The United States Pharmacopeia/The National Formulary). Such cellulose derivatives include, but are not limited to, methyl cellulose, carboxymethyl cellulose salt, carboxymethyl cellulose sodium, hydroxypropyl cellulose, methyl hydroxypropyl cellulose, hydroxypropyl methyl cellulose, cellulose acetate, ethyl cellulose, methyl hydroxyethyl cellulose, hydroxyethyl cellulose, and cellulose gum.

The gels of compositions of the present invention may further comprise one or more inorganic salts or salts of organic amines or amino acids in an amount effective to provide a gel or semi-gel with the desired viscosity. Sodium acetate is an exemplary salt that may be used for this purpose. Those skilled in the art will be capable of determining the appropriate quantity of such a salt to be added to a gel composition of the present invention. By way of example, however, sodium acetate concentrations in the range of about 0.01 to about 0.5% by weight of the gel have generally been found to be appropriate for providing gels of a suitable original and residual viscosity.

Low to medium viscosity cellulose based agents may be used in compositions of the present invention. Such agents have a lower number of substituents, such as methoxy, ethoxy-, hydroxy-propyl- and carboxy-substituents, attached to the cellulose backbone than high viscosity cellulose based agents. Some examples of the optional “non-cellulose based gels or semi-gels that are suitable for use in the compositions of the present invention may comprise dextran, polyvinyl alcohol, polyvinylacyrlates, poloxamer and polymeric mixtures thereof. In some instances, a higher concentration may be used, constituting from about 5.0 to about 15 wt. % or more of the formulation.

A buffered antiseptic surface preparation according to the present invention may contain additional pharmaceutically inactive substances, such as one or more solubilizing agents, such as polysorbate 20, polysorbate 40, polysorbate 60 or polysorbate 80. A buffered antiseptic surface preparation of the present invention may also contain a dispersant, such as lecithin or glycerine. Collagen may also be added. Preservatives may also be added but care should be taken so as not to significantly reduce the pH without additional buffers. Examples include methyl paraben, propyl paraben, sorbic acid (about 0.1%- 0.25%)/edetate disodium (about 0.025%- 0.5%); polyaminopropyl biguanide (about 0.00003%)/edetate disodium (about 0.025%); edetate disodium or EDTA (about 0.1%)/Polyquad® (polyquartenium-1, about 0.001%); sorbic acid (about 0.1%)/trisodium EDTA (about 0.25%); benzalkonium chloride, SofZia™ buffer system (borate, sorbitol, propylene glycol and zinc), sodium perborate, chlorobutanol, cetrimonium chloride, Purite™ (SOC, stabilized oxychloro-complex), polyhexamethylene biguanide, sodium bisulfite (0.05%), sodium metabisulfite (0.1%), sodium thiosulfate (0.1%) and ascorbic acid (0.1%).

The buffered surface preparations for use on the eye may be isotonic. The buffered surface preparations of the present invention may be isotonized by the use of suitable nonionic agents. Commonly, sorbitol or mannitol may be used for this purpose. Glycerol may also be used. The eye tolerates osmolarities in the range of 100-450 mOsmol/L.

Embodiments of the buffered surface preparations of the present invention may also comprise cyclodextrins, vitamin E, particularly in a solubilized form, and other antioxidants. Furthermore, the buffered surface preparations of the present invention may also comprise other ingredients, including sodium carbonate (from about 0.1% to about 5.0%), potassium chloride (from about 0.01% to about 1.0%), sodium citrate (from about 0.01% to about 5.0%), sodium thiosulfate (from about 0.01% to about 5.0%), sodium bisulfite, acetic acid, dextrose, magnesium chloride, alginic acid, and sodium borate.

Yet further embodiments of the buffered surface preparations of the present invention may further comprise a steroid, including but not limited to: hydrocortisone (from about 0.1% to about 10%), prednisone (from about 0.01% to about 10%), fluorometholone acetate (from about 0.01% to about 10%), dexamethasone sodium phosphate (from about 0.001% to about 1%), dexamethasone (from about 0.001% to about 1%), suprofen (from about 0.1% to about 10%), fluorometholone (from about 0.001% to about 1%), medrysone (from 20 about 0.1% to about 10%) and difluprednate (from about 0.01% to 1.0%.

Still further buffered surface preparations of the present invention may comprise, without limitation, betaxolol hydrochloride (from about 0.1% to about 10%), cyclopentolate hydrochloride (from about 0.1% to about 10%), p-phenylephrine hydrochloride (from about 0.1% to about 30%), epinephrine (from about 0.01% to about 20%), apraclonidine hydrochloride (from about 0.1% to about 10%), atropine sulfate (from about 0.1% to about 5%), carbachol (from about 0.1% to about 5%), pilocarpine hydrochloride (about 0.25%, 0.5%, 1%, 2%, 3%, 4%, 5%, 6%, 8%, and 10%), sulfacetamide sodium (from about 0.1% to about 30%), homatropine hydrobromide (from about 0.5% to about 10%), scopolamine hydrobromide (from about 0.1% to about 5%), tropicamide (from about 0.1% to about 5%), naphazolinchydrochloride (from about 0.01% to about 5%), tetrahydrozoline hydrochloride (from about 0.001% to about 5%), oxymetazoline hydrochloride (from about 0.001% to about 5%), ketorolac tromethamine (from about 0.001% to about 5%), levobunolol hydrochloride (from about 0.001% to about 5%), idoxuridine (from about 0.01% to about 2%), trimethoprim (from about 0.1 to about 5.0 mg/gm), dipivefrin hydrochloride (from about 0.01% to about 5%.), metipranolol (from about 0.01% to about 5%), trifluridine (from about 0.01% to about 5%), diclofenac sodium (from about 0.01% to about 5%), zinc isoflurophate (from about 0.01% to about 5 3%), demecarium bromide (from about 0.01% to about 5%), timolol malcate (from about 0.01% to about 5%), carteolol hydrochloride (from about 0.5 to about 25.0 mg/gm), and vidrabine (from about 0.1% to about 15%).

The buffered surface preparations of the present invention provide a novel combination of compounds in a formulation with benefits that heretofore were not obvious. The potential benefits include better sterility during ocular surface and ocular adnexa (eyelids and eyelashes) preparation, patient comfort, improved post-operative visual acuity, and an overall easier and quicker procedure.

Combining a buffered surface preparation of the present invention with an anesthetic as part of a kit, provides a one-step application method for providing a sterile field and anesthesia. Using a buffered and gelatinous surface preparation of the present invention facilitates the thorough coating of ocular structures with prolonged and improved contact time for bacterial inhibition, compared with conventional compositions and methods. In addition, with prolonged and improved contact time, use of the gelatinous surface preparation of the present invention can further limit the growth of bacteria that might be liberated from eyelid glands during the manipulation.

Beyond the examples given above in the field of ophthalmology, the attributes of the buffered surface preparation and methods of the present invention also improve medical treatment, surgical interventions, and wound care in other medical disciplines. As an additional method of the use of the buffered surface preparation of the present invention, a gel or semi-gel composition comprising antiseptic agents may be applied to the tympanic membrane prior to myringotomy procedures. In such an example, the tympanic membrane is an anatomic surface which must be incised as part of the planned treatment procedure. Because of its adherent properties, use of an optional gel or semi-gel with the buffered antiseptic agents provides better topical activity than liquid applications of similar components. Moreover, an aqueous gel or semi-gel composition is more easily removed by irrigation when desired from the tympanic membrane surface than would be the case with a non-aqueous gel or ointment composition.

As yet another method of the use of the buffered surface preparation of the present invention is application to a laceration, abrasion, burn, or other cutaneous wound prior to treatment. The buffered attributes induce less discomfort compared to the commonly used povidone-iodine, which is acidic in nature.

In still other methods of the use of the buffered surface preparation of the present invention, with or without an optional gel or semi-gel, is application to anatomic surfaces such as mucous membranes in the mouth during dental and oral surgical procedures including dental fillings, endodontic, orthodontic, periodontal, or dental implant treatments and surgery. Oral procedures require antiseptic preparation of an irregular surface where the improved contact time of a gelatinous material would be beneficial.

The buffered antiseptic surface preparations of the present invention for treatment of an anatomic surface comprise an effective amount of an antiseptic agent and sufficient buffering agent to provide a pH in the range of 6.0 to 8.5, preferably near a neutral pH. Optionally, the buffered surface preparation of the present invention includes an aqueous gel or semi-gel formulation. In certain embodiments, an effective amount of an anesthetic agent is added prior to use. Such compositions of the present invention find particular use in preparation for surgery or other invasive procedure.

Compositions of the present invention for treatment of an anatomic surface may comprise a steroid.

Compositions of the present invention for treatment of an anatomic surface may comprise an antibiotic.

Compositions of the present invention for treatment of an anatomic surface may comprise a preservative.

Methods for a treatment of an anatomic surface of the present invention comprise contacting an anatomic surface with a composition comprising an effective amount of an antiseptic agent buffered to a pH in the range of 6.0 to 8.5, preferably a near neutral pH. The composition may optionally contain a gel or semi-gel formulation. In certain embodiments, an effective amount of an anesthetic agent is added prior to use. Allowing said composition to remain in contact with said surface for a desired period of time follows so as to achieve the desired antiseptic treatment of said surface and where present, anesthetic treatment. Preliminary testing revealed that a contact time of 15 seconds is adequate to result in 100% kill of bacteria. An anatomic surface for treatment using methods of the present invention may comprise a cornea, a globe, a tympanic membrane, a mucous membrane, or a wound, such as a laceration, burn, or abrasion.

A method of the present invention provides antisepsis of an ocular surface that comprises: a) applying in an antiseptic agent to said surface buffered to a pH in the range of 6.0 to 8.5, optionally with a gel or semi-gel formulation and b) allowing said composition to remain in contact with said ocular surface for a desired period of time. Preferably at least 15 seconds. In an embodiment of this method, an anesthetic is added to the antiseptic agent prior to application and both antisepsis and anesthesia are achieved in one application.

Although the foregoing embodiments of the present invention have been described in some detail by way of illustration and example for purposes of clarity and understanding, it will be apparent to those skilled in the art that certain changes and modifications may be practiced within the spirit and scope of the present invention. Therefore, the descriptions presented herein should not be construed to limit the scope of the present invention, the essential features of which are set forth in the appended claims. The contents of copending U.S. patent application Ser. No. 15/868,357, filed Jan. 11, 2018, are incorporated by reference herein.

### Examples

### Preparation of Buffered Anatomic Solution w/o Gel

In the following examples a concentrated solution of the antiseptic is diluted with water and neutralized with an approximately 2.5 ml of sodium hydroxide (0.1N/water) to provide a buffered anatomic solution of approximately 100 ml. An alternative procedure is to dissolve the antiseptic, e.g. about 0.5 gm, in a large volume of water such as about 90% of the final volume and adjust the volume with water to approximately 100ml after buffering. Such a solution can be directly combined with a gel when used.

In the following examples sodium borate is added to the solution with sodium bicarbonate. The sodium borate can be introduced independently of sodium bicarbonate and may be introduced to the gel, when used, to render the gel isotonic.

The pH should be monitored during the addition of sodium hydroxide or sodium bicarbonate. Once a pH of 4.5 is reached, the sodium hydroxide or sodium bicarbonate solution should be added dropwise.

Once the final volume is achieved, the solution may be optionally filtered, such as with a 0.22 micron filter.

### Example 1

To a 100ml beaker is added 87.4 ml sterile water and 10 ml of 2% Chlorhexidine which are vigorously mixed with an overhead stirrer. Sodium bicarbonate of sufficient quantity and sodium borate 2.6 gm are added while stirring until dissolved to provide a pH in the range of 6.0-7.0. The solution remains stable for over 48 hours. The sample obtained is C0.2-SBI/SBO.

### Example 2

To a 100 mL beaker is added 77.4 ml sterile water and 20 ml of 4% Chlorhexidine which are vigorously mixed with an overhead stirrer. Sodium bicarbonate of sufficient quantity and sodium borate 2.6 gm are added while stirring until dissolved to provide a pH in the range of 6.0-7.0. The solution remains stable for over 48 hours. The sample obtained is C0.8-SBI/SBO.

### Example 3

To a 100mL beaker is added 72.4 ml sterile water and 25 ml of 5% Chlorhexidine which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is C1.25-SBI/SBO.

### Example 4

To a 100 ml beaker is added 47.4 ml of sterile water and 50 ml of 5% Chlorhexidine which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is C2.5-SBI/SBO.

### Example 5

To a 100 ml beaker is added 77.4 ml sterile water and 20 ml of 2% povidone-iodine which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is PI0.4-SBI/SBO.

### Example 6

To a 100 ml beaker is added 57.4 ml of sterile water and 40 ml of 4% povidone-iodine which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is PI1.6-SBI/SBO.

### Example 7

To a 100 ml beaker is added 47.4 ml of sterile water and 50 ml of 5% povidone-iodine which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is PI2.5-SBI/SBO.

### Example 8

To a 100 ml beaker is added 17.5 ml of sterile water and 80 ml of 8% povidone-iodine which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is PI6.4-SBI/SBO.

### Example 9

To a 100 ml beaker is added 95.4 ml of sterile water and 1 ml of 1% benzalkonium chloride which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is BK0.01-SBI/SBO.

### Example 10

To a 100 ml beaker is added 96.9 ml of sterile water and 0.5 ml of 1% benzalkonium chloride which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is BK0.005-SBI/SBO.

### Example 11

To a 100 mL beaker is added 96.9 ml of sterile water and 0.5 ml of 0.5% benzalkonium chloride which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is BK0.0025-SBI/SBO.

### Example 12

To a 100 mL beaker is added 96.4 ml of sterile water and 1 ml of 0.9% benzalkonium chloride which is vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is BK0.009-SBI/SBO.

### Example 13

To a 100 mL beaker is added 77.4 ml of sterile water and 20 ml of 2% chlorobutanol which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is CB0.4-SBI/SBO.

### Example 14

To a 100 mL beaker is added 92.4 ml of sterile water and 5 ml of 4% chlorobutanol which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is CB0.2-SBI/SBO.

### Example 15

To a 100 ml beaker is added 92.4 ml of sterile water and 5 ml of 4.5% chlorobutanol which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is CB0.23-SBI/SBO.

### Example 16

To a 100 ml beaker is added 90 ml of sterile water and 10 ml of 2% Chlorhexidine which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is C0.2-SH.

### Example 17

To a 100 ml beaker is added 60 ml of sterile water and 40 ml of 4% Chlorhexidine which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is C1.6-SH.

### Example 18

To a 100 mL beaker is added 50 ml of sterile water and 50 ml of 5% Chlorhexidine which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is C2.5-SH.

### Example 19

To a 100 ml beaker is added 55 ml of sterile water and 45 ml of 5% Chlorhexidine which is vigorously mixed with an overhead stirrer. Sodium hydroxide of sufficient quantity is slowly added while stirring until dissolved to provide a pH in the range of 6.5-7.5. The solution remains stable for over 48 hours. The sample obtained is C2.25-SH.

### Example 20

To a 100 mL beaker is added 80 ml of sterile water and 20 ml of 2% povidone-iodine which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is PI0.2-SH.

### Example 21

To a 100 ml beaker is added 60 ml of sterile water and 40 ml of 4% povidone-iodine which are vigorously? mixed with an overhead stirrer. Sodium hydroxide of sufficient quantity is slowly added while stirring until dissolved to provide a pH in the range of 6.5-7.5. The solution remains stable for over 48 hours. The sample obtained is PI1.6-SH.

### Example 22

To a 100 ml beaker is added 50 ml of sterile water and 50 ml of 5% povidone-iodine which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is PI2.5-SH.

### Example 23

To a 100 ml beaker is added 10 ml of sterile water and 90 ml of 9% povidone-iodine which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is PI8.1-SH.

### Example 24

To a 100 ml beaker is added 99 ml of sterile water and 1 ml of 1% benzalkonium chloride which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is BK0.01-SH.

### Example 25

To a 100 ml beaker is added 99.5 ml of sterile water and 0.5 ml of 1% benzalkonium chloride which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is BK0.005-SH.

### Example 26

To a 100 ml beaker is added 99.6 ml of sterile water and 0.4 ml of 2% benzalkonium chloride which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is BK0.008-SH.

### Example 27

To a 100 ml beaker is added 10 ml of sterile water and 90 ml of 9% benzalkonium chloride which is vigorously mixed with an overhead stirrer. Sodium hydroxide of sufficient quantity is slowly added while stirring until dissolved to provide a pH in the range of 6.5-7.5. The solution remains stable for over 48 hours. The sample obtained is BK8.1-SH.

### Example 28

To a 100 ml beaker is added 80 ml of sterile water and 20 ml of 2% chlorobutanol which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is CB0.4-SH.

### Example 29

To a 100 ml beaker is added 92 ml of sterile water and 8 ml of 4% chlorobutanol which are vigorously mixed with an overhead stirrer. Sodium hydroxide of sufficient quantity is slowly added while stirring until dissolved to provide a pH in the range of 6.5-7.5. The solution remains stable for over 48 hours/6 months hours. The sample obtained is CB0.32-SH.

### Example 30

To a 100 ml beaker is added 90 ml of sterile water and 10 ml of 4.5% chlorobutanol which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is CB0.45-SH.

### Preparation of Buffered Anatomic Solution w/Gel

### Example 31

To a 100 ml beaker is added 37.4 ml of sterile water, 10 ml of 2% Chlorhexidine and 50 ml of 5% methylcellulose, which is vigorously stirred with an overhead stirrer. Sodium bicarbonate of sufficient quantity and sodium borate 2.6 gm are slowly added while stirring until dissolved to provide a pH in the range of 6.0-7.0. The viscosity of the solution is between 10,000-50,000 cps at 25° C. The solution remains stable for over 48 hours. The sample obtained is C0.1-MC2.5-SBI/SBO.

### Example 32

To a 100 ml beaker is added 27.4 ml g sterile water, 20 ml of 4% Chlorhexidine and 50ml of 5% methylcellulose, which is vigorously stirred with an overhead stirrer. Sodium bicarbonate of sufficient quantity and sodium borate 2.6 gm are slowly added while stirring until dissolved to provide a pH in the range of 6.0-7.0. The viscosity of the solution is between 10,000-50,000 cps at 25° C. The solution remains stable for over 48 hours. The sample obtained is C0.4-MC2.5-SBI/SBO.

### Example 33

To a 100 mL beaker is added 17.4 ml of sterile water, 20 ml of 4% Chlorhexidine and 60 ml of 6% methylcellulose, which is vigorously stirred with an overhead stirrer. Sodium bicarbonate of sufficient quantity and sodium borate 2.6 gm are slowly added while stirring until dissolved to provide a pH in the range of 6.0-7.0. The viscosity of the solution is between 10,000-50,000 cps at 25° C. The solution remains stable for over 48 hours. The sample obtained is C0.4-MC3.6-SBI/SBO

### Example 34

To a 100 ml beaker is added 17.4 ml of sterile water, 30 ml of 6% Chlorhexidine and 50 ml of 5% methylcellulose, which is vigorously stirred with an overhead stirrer. Sodium bicarbonate of sufficient quantity and sodium borate 2.6 gm are slowly added while stirring until dissolved to provide a pH in the range of 6.0-7.0. The viscosity of the solution is between 10,000-50,000 cps at 25° C. The solution remains stable for over 48 hours. The sample obtained is C1.8-MC2.5-SBI/SBO.

### Example 35

To a 100 mL beaker is added 7.4 ml of sterile water, 40 ml of 8% Chlorhexidine and 50 ml of 5% methylcellulose, which is vigorously stirred with an overhead stirrer. Sodium bicarbonate of sufficient quantity and sodium borate 2.6 gm are slowly added while stirring until dissolved to provide a pH in the range of 6.0-7.0. The viscosity of the solution is between 10,000-50,000 cps at 25° C. The solution remains stable for over 48 hours. The sample obtained is C3.2-MC2.5-SBI/SBO.

### Example 36

To a 100 mL beaker is added 0 ml sterile water, 50 ml of 5% Chlorhexidine and 50 ml of 20% methylcellulose, which is vigorously stirred with an overhead stirrer. Sodium bicarbonate of sufficient quantity and sodium borate 2.6 gm are slowly added while stirring until dissolved to provide a pH in the range of 6.0-7.0. The viscosity of the solution is between 10,000-50,000 cps at 25° C. The solution remains stable for over 48 hours. The sample obtained is C2.5-MC10-SBI/SBO.

### Example 37

To a 100 mL beaker is added 27.4 ml of sterile water, 20 ml of 2% povidone-iodine and 50 ml of 5% methylcellulose, which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is PI0.4-MC2.5-SBI/SBO.

### Example 38

To a 100 ml beaker is added 57.4 ml of sterile water, 30 ml of 4% povidone-iodine and 10 ml of 5% methylcellulose, which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is PI1.2-MC0.5-SBI/SBO.

### Example 39

To a 100 ml mL beaker is added 27.4 ml of sterile water, 60 ml of 4% povidone-iodine and 10 ml of 10% methylcellulose, which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is PI2.4-MC1.0-SBI/SBO.

### Example 40

To a 100 ml beaker is added 7.4 ml of sterile water, 40 ml of 4% povidone-iodine and 50 ml of 5% methylcellulose, which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is PI1.6-MC2.5-SBI/SBO.

### Example 41

To a 100 ml beaker is added 7.4 ml of sterile water, 80 ml of 8% povidone-iodine and 10 ml of 5% methylcellulose, which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is PI3.6-MC0.5-SBI/SBO.

### Example 42

To a 100 ml beaker is added 0 ml sterile water, 50 ml of 10% povidone-iodine and 50 ml of 20% methylcellulose, which is vigorously stirred with an overhead stirrer. Sodium bicarbonate of sufficient quantity and sodium borate 2.6 gm are slowly added while stirring until dissolved to provide a pH in the range of 6.0-7.0. The viscosity of the solution is between 10,000-50,000 cps at 25° C. The solution remains stable for over 48 hours. The sample obtained is PI5-MC10-SBI/SBO.

### Example 43

To a 100 ml beaker is added 47.2 ml of sterile water, 0.2 ml of 2% benzalkonium chloride and 50 ml of 5% methylcellulose which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is BK0.004-MC2.5-SBI/SBO.

### Example 44

To a 100 mL beaker is added 47.2 ml of sterile water, 0.2 ml of 4% benzalkonium chloride and 50 ml of 5% methylcellulose which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is BK0.008-MC2.5-SBI/SBO.

### Example 45

To a 100 ml beaker is added 87.2 ml of sterile water, 0.2 ml of 2% benzalkonium chloride and 10 ml of 10% methylcellulose gel which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is BK0.004-MC1.0-SBI/SBO.

### Example 46

To a 100 ml beaker is added 87.3 ml of sterile water, 0.1 ml of 5% benzalkonium chloride and 10 ml of 1% methylcellulose which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is BK0.005-MC0.1-SBI/SBO.

### Example 47

To a 100 ml beaker is added 87.2 ml of sterile water 0.2 ml of 2% benzalkonium chloride and 10 ml of 1% methylcellulose which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is BK0.004-MC0.1-SBI/SBO.

### Example 48

To a 100 ml beaker is added 49.75 ml sterile water, 0.25 ml of 1% benzalkonium chloride and 50 ml of 20% methylcellulose, which is vigorously stirred with an overhead stirrer. Sodium bicarbonate of sufficient quantity and sodium borate 2.6 gm are slowly added while stirring until dissolved to provide a pH in the range of 6.0-7.0. The viscosity of the solution is between 10,000-50,000 cps at 25° C. The solution remains stable for over 48 hours. The sample obtained is BK.0025-MC10-SBI/SBO.

### Example 49

To a 100 ml beaker is added 27.4 ml of sterile water, 20 ml of 2% chlorobutanol and 50 ml of 5% methylcellulose which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is CB0.4-MC2.5-SBI/SBO.

### Example 50

To a 100 mL beaker is added 79.4 ml of sterile water, 8 ml of 4% chlorobutanol and 10 ml of 1% methylcellulose which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is CB0.32-MC0.1-SBI/SBO.

### Example 51

To a 100 ml beaker is added 27.4 ml of sterile water, 60 ml of 1% chlorobutanol and 10 ml of 1% methylcellulose which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is CB0.6-MC0.1-SBI/SBO.

### Example 52

To a 100 ml beaker is added 27.4 ml of sterile water, 20 ml of 1% chlorobutanol and 50 ml of 5% methylcellulose which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is CB0.2-MC2.5-SBI/SBO.

### Example 53

To a 100 ml beaker is added 67.4 ml of sterile water, 20 ml of 1% chlorobutanol and 10 ml of 1% methylcellulose gel which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is CB0.1-MC2.5-SBI/SBO.

### Example 54

To a 100 ml beaker is added 45 ml sterile water, 5 ml of 10% chlorobutanol and 50 ml of 20% methylcellulose, which is vigorously stirred with an overhead stirrer. Sodium bicarbonate of sufficient quantity and sodium borate 2.6 gm are slowly added while stirring until dissolved to provide a pH in the range of 6.0-7.0. The viscosity of the solution is between 10,000-50,000 cps at 25° C. The solution remains stable for over 48 hours. The sample obtained is CB0.5-MC10-SBI/SBO.

### Example 55

To a 100 ml beaker is added 62.4 ml of sterile water, 10 ml of 2% Chlorhexidine and 25 ml of 5% poloxamer gel, which is vigorously stirred with an overhead stirrer. Sodium bicarbonate of sufficient quantity and sodium borate 2.6 gm are slowly added while stirring until dissolved to provide a pH in the range of 6.0-7.0. The viscosity of the solution is between 10,000-50,000 cps at 25° C. The solution remains stable for over 48 hours. The sample obtained is C0.2-P1.25-SBI/SBO.

### Example 56

To a 100 ml beaker is added 55 ml of sterile water, 20 ml of 4% Chlorhexidine and 25 ml of 5% poloxamer gel, which is vigorously stirred with an overhead stirrer. Sodium bicarbonate of sufficient quantity and sodium borate 2.6 gm are slowly added while stirring until dissolved to provide a pH in the range of 6.0-7.0. The viscosity of the solution is between 10,000-50,000 cps at 25° C. The solution remains stable for over 48 hours. The sample obtained is C0.8-P1.25-SBI/SBO.

### Example 57

To a 100 ml beaker is added 27.4 ml g sterile water, 20 ml of 4% Chlorhexidine and 50 ml of 10% poloxamer gel, which is vigorously stirred with an overhead stirrer. Sodium bicarbonate of sufficient quantity and sodium borate 2.6 gm are slowly added while stirring until dissolved to provide a pH in the range of 6.0-7.0. The viscosity of the solution is between 10,000-50,000 cps at 25° C. The solution remains stable for over 48 hours. The sample obtained is C0.8-P5-SBI/SBO.

### Example 58

To a 100 ml beaker is added 42.4 ml of sterile water, 30 ml of 6% Chlorhexidine and 25 ml of 5% poloxamer gel, which is vigorously stirred with an overhead stirrer. Sodium bicarbonate of sufficient quantity and sodium borate 2.6 gm are slowly added while stirring until dissolved to provide a pH in the range of 6.0-7.0. The viscosity of the solution is between 10,000-50,000 cps at 25° C. The solution remains stable for over 48 hours. The sample obtained is C1.8-P1.25-SBI/SBO.

### Example 59

To a 100 ml beaker is added 32.4 ml of sterile water, 40 ml of 10% Chlorhexidine and 25 ml of 5% poloxamer gel, which is vigorously stirred with an overhead stirrer. Sodium bicarbonate of sufficient quantity and sodium borate 2.6 gm are slowly added while stirring until dissolved to provide a pH in the range of 6.0-7.0. The viscosity of the solution is between 10,000-50,000 cps at 25° C. The solution remains stable for over 48 hours. The sample obtained is C4-P1.25-SBI/SBO.

### Example 60

To a 100 ml beaker is added 0 ml sterile water, 50 ml of 5% Chlorhexidine and 50 ml of 20% poloxamer gel, which is vigorously stirred with an overhead stirrer. Sodium bicarbonate of sufficient quantity and sodium borate 2.6 gm are slowly added while stirring until dissolved to provide a pH in the range of 6.0-7.0. The viscosity of the solution is between 10,000-50,000 cps at 25° C. The solution remains stable for over 48 hours. The sample obtained is C2.5-P10-SBI/SBO.

### Example 61

To a 100 ml beaker is added 52.4 ml of sterile water, 20 ml of 2% povidone-iodine and 25 ml of 5% poloxamer gel, which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is PI0.4-P1.25-SBI/SBO.

### Example 62

To a 100 ml beaker is added 12.4 ml of sterile water, 60 ml of 4% povidone-iodine and 25 ml of 5% poloxamer gel, which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is PI2.4-P1.25-SBI/SBO.

### Example 63

To a 100 ml beaker is added 32.4 ml of sterile water, 60 ml of 4% povidone-iodine and 5 ml of 1% poloxamer gel, which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours/6 months hours. The sample obtained is PI2.4-P0.05-SBI/SBO.

### Example 64

To a 100 ml beaker is added 17.4 ml of sterile water, 75 ml of 5% povidone-iodine and 5 ml of 1% poloxamer gel which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is PI3.75-P0.05-SBI/SBO.

### Example 65

To a 100 ml beaker is added 22.4 ml of sterile water, 50 ml of 5% povidone-iodine and 25 ml of 5% poloxamer gel which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours/6 months hours. The sample obtained is PI2.5-P1.25-SBI/SBO.

### Example 66

To a 100 ml beaker is added 0 ml sterile water, 50 ml of 10% povidone-iodine and 50 ml of 20% poloxamer gel, which is vigorously stirred with an overhead stirrer. Sodium bicarbonate of sufficient quantity and sodium borate 2.6 gm are slowly added while stirring until dissolved to provide a pH in the range of 6.0-7.0. The viscosity of the solution is between 10,000-50,000 cps at 25° C. The solution remains stable for over 48 hours. The sample obtained is PI5-P10-SBI/SBO.

### Example 67

To a 100 ml beaker is added 72.2 ml of sterile water, 0.2 ml of 2% benzalkonium chloride and 25 ml of 5% poloxamer gel which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is BK0.004-P1.25-SBI/SBO.

### Example 68

To a 100 ml beaker is added 72.4 ml of sterile water, 0.4 ml of 2% benzalkonium chloride and 25 ml of 5% poloxamer gel which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is BK0.008-P1.25-SBI/SBO.

### Example 69

To a 100 mL beaker is added 71.9 ml of sterile water, 0.5 ml of 2% benzalkonium chloride and 25 ml of 5% poloxamer gel which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is BK0.01-P1.25-SBI/SBO.

### Example 70

To a 100 ml beaker is added 72.3 ml of sterile water, 0.1 ml of 7% benzalkonium chloride and 25 ml of 5% poloxamer gel which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is BK0.007-P1.25-SBI/SBO.

### Example 71

To a 100 mL beaker is added 47.1 ml sterile water, 0.3 ml of 3% benzalkonium chloride and 50 ml of 20% poloxamer gel, which is vigorously stirred with an overhead stirrer. Sodium bicarbonate of sufficient quantity and sodium borate 2.6 gm are slowly added while stirring until dissolved to provide a pH in the range of 6.0-7.0. The viscosity of the solution is between 10,000-50,000 cps at 25° C. The solution remains stable for over 48 hours. The sample obtained is BK0.009-P10-SBI/SBO.

### Example 72

To a 100 ml beaker is added 52.4 ml of sterile water, 20 ml of 2% chlorobutanol and 25 ml of 5% poloxamer gel which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is CB0.4-P1.25-SBI/SBO.

### Example 73

To a 100 ml beaker is added 42.4 ml of sterile water, 30 ml of 1% chlorobutanol and 25 ml of 5% poloxamer gel which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is CB0.3-P1.25-SBI/SBO.

### Example 74

To a 100 ml beaker is added 52.4 ml of sterile water, 40 ml of 1% chlorobutanol and 5 ml of 1% poloxamer gel which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is CB0.04-P0.05-SBI/SBO.

### Example 75

To a 100 mL beaker is added 62.4 ml of sterile water, 10 ml of 5% chlorobutanol and 25 ml of 5% poloxamer gel which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is CB0.05-P1.25-SBI/SBO.

### Example 76

To a 100 ml beaker is added 72.4 ml of sterile water, 20 ml of 10% chlorobutanol and 5 ml of 1% poloxamer gel which are vigorously mixed with an overhead stirrer. Sodium bicarbonate of sufficient quantity and sodium borate 2.6 gm are slowly added while stirring until dissolved to provide a pH in the range of 6.0-7.0. The solution remains stable for over 48 hours. The sample obtained is CB0.2-P0.05-SBI/SBO.

### Example 77

To a 100 ml beaker is added 45 ml sterile water, 5 ml of 10% chlorobutanol and 50 ml of 20% poloxamer gel, which is vigorously stirred with an overhead stirrer. Sodium bicarbonate of sufficient quantity and sodium borate 2.6 gm are slowly added while stirring until dissolved to provide a pH in the range of 6.0-7.0. The viscosity of the solution is between 10,000-50,000 cps at 25° C. The solution remains stable for over 48 hours. The sample obtained is CB0.5-P10-SBI/SBO.

### Example 78

To a 100 ml beaker is added 40 ml of sterile water, 10 ml of 2% Chlorhexidine and 50 ml of 5% methylcellulose gel, which is vigorously stirred with an overhead stirrer. Sodium hydroxide of sufficient quantity is slowly added while stirring until dissolved to provide a pH in the range of 6.5-7.5. The viscosity of the solution is between 10,000-50,000 cps at 25° C. The solution remains stable for over 48 hours. The sample obtained is C0.2-MC1.25-SH.

### Example 79

To a 100 ml beaker is added 30 ml of sterile water, 20 ml of 4% Chlorhexidine and 50 ml of 5% methylcellulose gel, which is vigorously stirred with an overhead stirrer. Sodium hydroxide of sufficient quantity is slowly added while stirring until dissolved to provide a pH in the range of 6.5-7.5. The viscosity of the solution is between 10,000-50,000 cps at 25° C. The solution remains stable for over 48 hours. The sample obtained is C0.8-MC1.25-SH.

### Example 80

To a 100 ml beaker is added 70 ml of sterile water, 20 ml of 4% Chlorhexidine and 10 ml of 1% methylcellulose gel, which is vigorously stirred with an overhead stirrer. Sodium hydroxide of sufficient quantity is slowly added while stirring until dissolved to provide a pH in the range of 6.5-7.5. The viscosity of the solution is between 10,000-50,000 cps at 25° C. The solution remains stable for over 48 hours. The sample obtained is C0.8-MC0.1-SH.

### Example 81

To a 100 ml beaker is added 20 ml of sterile water, 30 ml of 6% Chlorhexidine and 50 ml of 5% methylcellulose gel, which is vigorously stirred with an overhead stirrer. Sodium hydroxide of sufficient quantity is slowly added while stirring until dissolved to provide a pH in the range of 6.5-7.5. The viscosity of the solution is between 10,000-50,000 cps at 25° C. The solution remains stable for over 48 hours. The sample obtained is C1.8-MC1.25-SH.

### Example 82

To a 100 ml beaker is added 40 ml of sterile water, 50 ml of 5% Chlorhexidine and 10 ml of 1% methylcellulose gel, which is vigorously stirred with an overhead stirrer. Sodium hydroxide of sufficient quantity is slowly added while stirring until dissolved to provide a pH in the range of 6.5-7.5. The viscosity of the solution is between 10,000-50,000 cps at 25° C. The solution remains stable for over 48 hours. The sample obtained is C2.5-MC0.1-SH.

### Example 83

To a 100 ml beaker is added 25 ml sterile water, 25 ml of 10% Chlorhexidine and 50 ml of 20% methylcellulose gel, which is vigorously stirred with an overhead stirrer. Sodium hydroxide of sufficient quantity is slowly added while stirring until dissolved to provide a pH in the range of 6.0-7.0. The viscosity of the solution is between 10,000-50,000 cps at 25° C. The solution remains stable for over 48 hours. The sample obtained is C2.5-MC10-SH.

### Example 84

To a 100 ml beaker is added 30 ml of sterile water, 20 ml of 2% povidone-iodine and 50 ml of 5% methylcellulose gel, which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is PI.4-MC1.25-SH.

### Example 85

To a 100 ml beaker is added 30 ml of sterile water, 60 ml of 4% povidone-iodine and 10 ml of 1% methylcellulose gel, which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is PI2.4-MC0.1-SH.

### Example 86

To a 100 ml beaker is added 20 ml of sterile water, 30 ml of 2% povidone-iodine and 50 ml of 5% methylcellulose gel, which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is PI.6-MC1.25-SH.

### Example 87

To a 100 ml beaker is added 0 ml sterile water, 50 ml of 5% povidone-iodine and 50 ml of 5% methylcellulose gel which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is PI2.5-MC2.5-SH.

### Example 88

To a 100 ml beaker is added 20 ml of sterile water, 60 ml of 6% povidone-iodine and 20 ml of 2% methylcellulose gel which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is PI3.6-MC0.4-SH.

### Example 89

To a 100 mL beaker is added 0 ml sterile water, 50 ml of 10% povidone-iodine and 50 ml of 20% methylcellulose gel, which is vigorously stirred with an overhead stirrer. Sodium hydroxide of sufficient quantity is slowly added while stirring until dissolved to provide a pH in the range of 6.0-7.0. The viscosity of the solution is between 10,000-50,000 cps at 25° C. The solution remains stable for over 48 hours. The sample obtained is PI5-MC10-SH.

### Example 90

To a 100 ml beaker is added 49.5 ml of sterile water, 0.5 ml of 2% benzalkonium chloride and 50 ml of 5% methylcellulose gel which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is BK0.01-MC1.25-SH.

### Example 91

To a 100 ml beaker is added 46.6 ml of sterile water, 0.4 ml of 2% benzalkonium chloride and 50 ml of 5% methylcellulose gel which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is BK0.008-MC1.25-SH.

### Example 92

To a 100 mL beaker is added 89.9 ml of sterile water, 0.1 ml of 4% benzalkonium chloride and 10 ml of 1% methylcellulose gel which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is BK0.004-MC0.1-SH.

### Example 93

To a 100 ml beaker is added 49.5 ml of sterile water, 0.2 ml of 5% benzalkonium chloride and 50 ml of 5% methylcellulose gel which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is BK0.01-MC2.5-SH.

### Example 94

To a 100 ml beaker is added 89 ml of sterile water 1 ml of 1% benzalkonium chloride and 10 ml of 1% methylcellulose gel which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is BK0.01-MC0.1-SH.

### Example 95

To a 100 ml beaker is added 49.2 ml sterile water, 0.8 ml of 1% benzalkonium chloride and 50 ml of 20% methylcellulose gel, which is vigorously stirred with an overhead stirrer. Sodium hydroxide of sufficient quantity is slowly added while stirring until dissolved to provide a pH in the range of 6.0-7.0. The viscosity of the solution is between 10,000-50,000 cps at 25° C. The solution remains stable for over 48 hours. The sample obtained is BK0.008-MC10-SH.

### Example 96

To a 100 mL beaker is added 30 ml of sterile water, 20 ml of 2% chlorobutanol and 50 ml of 5% methylcellulose gel which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is CB0.4-MC2.5-SH.

### Example 97

To a 100 ml beaker is added 82 ml of sterile water, 8 ml of 4% chlorobutanol and 10 ml of 1% methylcellulose gel which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is CB0.32-MC0.1-SH.

### Example 98

To a 100 mL beaker is added 40 ml of sterile water, 40 ml of 1% chlorobutanol and 20 ml of 2% methylcellulose gel which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is CB0.4-MC0.4-SH.

### Example 99

To a 100 ml beaker is added 60 ml of sterile water, 20 ml of 1% chlorobutanol and 20 ml of 2% methylcellulose gel, which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is CB0.2-MC0.4-SH.

### Example 100

To a 100 ml beaker is added 40 ml of sterile water, 30 ml of 1% chlorobutanol and 30 ml of 3% methylcellulose gel which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is CB0.3-MC0.9-SH.

### Example 101

To a 100 mL beaker is added 45 ml sterile water, 5 ml of 10% chlorobutanol and 50 ml of 20% methylcellulose gel, which is vigorously stirred with an overhead stirrer. Sodium hydroxide of sufficient quantity is slowly added while stirring until dissolved to provide a pH in the range of 6.0-7.0. The viscosity of the solution is between 10,000-50,000 cps at 25° C. The solution remains stable for over 48 hours. The sample obtained is CB0.5-MC10-SH.

### Example 102

To a 100 ml beaker is added 65 ml of sterile water, 10 ml of 2% Chlorhexidine and 25 ml of 5% poloxamer gel, which is vigorously stirred with an overhead stirrer. Sodium hydroxide of sufficient quantity is slowly added while stirring until dissolved to provide a pH in the range of 6.5-7.5. The viscosity of the solution is between 10,000-50,000 cps at 25° C. The solution remains stable for over 48 hours. The sample obtained is C0.2-P1.25-SH.

### Example 103

To a 100 mL beaker is added 55 ml of sterile water, 20 ml of 4% Chlorhexidine and 25 ml of 5% poloxamer gel, which is vigorously stirred with an overhead stirrer. Sodium hydroxide of sufficient quantity is slowly added while stirring until dissolved to provide a pH in the range of 6.5-7.5. The viscosity of the solution is between 10,000-50,000 cps at 25° C. The solution remains stable for over 48 hours. The sample obtained is C0.8-P1.25-SH.

### Example 104

To a 100 ml beaker is added 30 ml of sterile water, 20 ml of 4% Chlorhexidine and 50 ml of 10% poloxamer gel, which is vigorously stirred with an overhead stirrer. Sodium hydroxide of sufficient quantity is slowly added while stirring until dissolved to provide a pH in the range of 6.5-7.5. The viscosity of the solution is between 10,000-50,000 cps at 25° C. The solution remains stable for over 48 hours. The sample obtained is C0.8-P5-SH.

### Example 105

To a 100 mL beaker is added 45 ml of sterile water, 30 ml of 6% Chlorhexidine and 25 ml of 5% poloxamer gel, which is vigorously stirred with an overhead stirrer. Sodium hydroxide of sufficient quantity is slowly added while stirring until dissolved to provide a pH in the range of 6.5-7.5. The viscosity of the solution is between 10,000-50,000 cps at 25° C. The solution remains stable for over 48 hours. The sample obtained is C1.8-P1.25-SH.

### Example 106

To a 100 ml beaker is added 25 ml of sterile water, 50 ml of 10% Chlorhexidine and 25 ml of 5% poloxamer gel, which is vigorously stirred with an overhead stirrer. Sodium hydroxide of sufficient quantity is slowly added while stirring until dissolved to provide a pH in the range of 6.5-7.5. The viscosity of the solution is between 10,000-50,000 cps at 25° C. The solution remains stable for over 48 hours. The sample obtained is C1-P1.25-SH.

### Example 107

To a 100 ml beaker is added 0 ml sterile water, 50 ml of 5% Chlorhexidine and 50 ml of 20% poloxamer gel, which is vigorously stirred with an overhead stirrer. Sodium hydroxide of sufficient quantity is slowly added while stirring until dissolved to provide a pH in the range of 6.0-7.0. The viscosity of the solution is between 10,000-50,000 cps at 25° C. The solution remains stable for over 48 hours. The sample obtained is C2.5-P10-SH.

### Example 108

To a 100 mL beaker is added 55 ml of sterile water, 20 ml of 2% povidone-iodine and 25 ml of 5% poloxamer gel, which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is PI0.4-P1.25-SH.

### Example 109

To a 100 ml beaker is added 15 ml of sterile water, 60 ml of 4% povidone-iodine and 25 ml of 5% poloxamer gel, which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is PI2.4-P1.25-SH.

### Example 110

To a 100 mL beaker is added 35 ml of sterile water, 60 ml of 4% povidone-iodine and 5 ml of 1% poloxamer gel, which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is PI2.4-P0.05-SH.

### Example 111

To a 100 ml beaker is added 25 ml of sterile water, 50 ml of 5% povidone-iodine and 25 ml of 5% poloxamer gel which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is PI2.5-P1.25-SH.

### Example 112

To a 100 mL beaker is added 15 ml of sterile water, 60 ml of 6% povidone-iodine and 25 ml of 5% poloxamer gel which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is PI3.6-P1.25-SH.

### Example 113

To a 100 ml beaker is added 0 ml sterile water, 50 ml of 10% % povidone-iodine and 50 ml of 20% poloxamer gel, which is vigorously stirred with an overhead stirrer. Sodium hydroxide of sufficient quantity is slowly added while stirring until dissolved to provide a pH in the range of 6.0-7.0. The viscosity of the solution is between 10,000-50,000 cps at 25° C. The solution remains stable for over 48 hours. The sample obtained is PI5-P10-SH.

### Example 114

To a 100 ml beaker is added 74.5 ml of sterile water, 0.5 ml of 2% benzalkonium chloride and 25 ml of 5% poloxamer gel which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is BK0.01-P1.25-SH.

### Example 115

To a 100 ml beaker is added 74.6 ml of sterile water, 0.4 ml of 1% benzalkonium chloride and 25 ml of 5% poloxamer gel which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is BK0.004-P1.25-SH.

### Example 116

To a 100 ml beaker is added 49 ml of sterile water, 1 ml of 1% benzalkonium chloride and 50 ml of 10% poloxamer gel which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is BK0.01-P5-SH.

### Example 117

To a 100 mL beaker is added 74.5 ml of sterile water, 0.5 ml of 2% benzalkonium chloride and 25 ml of 5% poloxamer gel which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is BK0.01-P1.25-SH.

### Example 118

To a 100 mL beaker is added 74.5 ml of sterile water, 0.5 ml of 1% benzalkonium and 25 ml of 5% poloxamer gel which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is BK0.005-P1.25-SH.

### Example 119

To a 100 ml beaker is added 49 ml sterile water, 1 ml of 1% benzalkonium and 50 ml of 20% poloxamer gel, which is vigorously stirred with an overhead stirrer. Sodium hydroxide of sufficient quantity is slowly added while stirring until dissolved to provide a pH in the range of 6.0-7.0. The viscosity of the solution is between 10,000-50,000 cps at 25° C. The solution remains stable for over 48 hours. The sample obtained is BK0.01-P10-SH.

### Example 120

To a 100 ml beaker is added 55 ml of sterile water, 20 ml of 2% chlorobutanol and 25 ml of 5% poloxamer gel which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is CB0.4-P1.25-SH.

### Example 121

To a 100 ml beaker is added 45 ml of sterile water, 30 ml of 1% chlorobutanol and 25 ml of 5% poloxamer gel which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is CB0.3-P1.25-SH.

### Example 122

To a 100 ml beaker is added 87 ml of sterile water, 8 ml of 4% chlorobutanol and 5 ml of 1% poloxamer gel which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is CB0.32-P0.05-SH.

### Example 124

To a 100 ml beaker is added 55 ml of sterile water, 20 ml of 1% chlorobutanol and 25 ml of 5% poloxamer gel which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is CB0.2-P1.25-SH.

### Example 125

To a 100 ml beaker is added 75 ml of sterile water, 20 ml of 1% chlorobutanol and 5 ml of 1% poloxamer gel which are vigorously mixed with an overhead stirrer. Sodium hydroxide of sufficient quantity is slowly added while stirring until dissolved to provide a pH in the range of 6.5-7.5. The solution remains stable for over 48 hours. The sample obtained is CB0.5-P0.05-SH.

### Example 126

To a 100 mL beaker is added 45 ml sterile water, 5 ml of 10% chlorobutanol and 50 ml of 20% poloxamer gel, which is vigorously stirred with an overhead stirrer. Sodium hydroxide of sufficient quantity is slowly added while stirring until dissolved to provide a pH in the range of 6.0-7.0. The viscosity of the solution is between 10,000-50,000 cps at 25° C. The solution remains stable for over 48 hours. The sample obtained is CB0.5-P10-SH.

In a similar manner to the methods described above, the following samples are prepared using the components indicated:
C0.1-SBI/SBO, C0.3-SBI/SBO, C0.4-SBI/SBO, C0.5-SBI/SBO, C0.6-SBI/SBO, C0.7-SBI/SBO, C0.9-SBI/SBO, C1-SBI/SBO, C1.5-SBI/SBO, C2-SBI/SBO, C2.5-SBI/SBO, C3-SBI/SBO, C3.5-SBI/SBO, C4-SBI/SBO, C4.5-SBI/SBO, C6-SBI/SBO, C7-SBI/SBO, C8-SBI/SBO, C9-SBI/SBO, C10-SBI/SBO;
PI0.1-SBI/SBO, PI0.2-SBI/SBO, PI0.3-SBI/SBO, PI0.5-SBI/SBO, PI0.6-SBI/SBO, PI0.7-SBI/SBO, PI0.8-SBI/SBO, PI0.9-SBI/SBO, PI1-SBI/SBO, PI1.25-SBI/SBO, PI1.5-SBI/SBO, PI1.75-SBI/SBO, PI2-SBI/SBO, PI2.25-SBI/SBO, PI2.75-SBI/SBO, PI3-SBI/SBO, PI3.25-SBI/SBO, PI3.5-SBI/SBO, PI4-SBI/SBO, PI4.5-SBI/SBO, PI5-SBI/SBO, PI5.5-SBI/SBO, PI6-SBI/SBO, P7-SBI/SBO, PI8-SBI/SBO, PI9-SBI/SBO, PI10-SBI/SBO;
BK0.1-SBI/SBO, BK0.2-SBI/SBO, BK0.3-SBI/SBO, BK0.5-SBI/SBO, BK0.6-SBI/SBO, BK0.7-SBI/SBO, BK0.8-SBI/SBO, BK0.9-SBI/SBO, BK1-SBI/SBO, BK1.25-SBI/SBO, BK1.75-SBI/SBO, BK2-SBI/SBO, BK2.75-SBI/SBO, BK3-SBI/SBO, BK3.5-SBI/SBO, BK4-SBI/SBO, BK5-SBI/SBO, BK6-SBI/SBO, BK7-SBI/SBO, BK8-SBI/SBO, BK9-SBI/SBO, BK10-SBI/SBO;
CB0.1-SBI/SBO, CB0.2-SBI/SBO, CB0.3-SBI/SBO, CB0.4-SBI/SBO, CB0.5-SBI/SBO, CB0.6-SBI/SBO, CB0.7-SBI/SBO, CB0.9-SBI/SBO, CB1-SBI/SBO, CB1.25-SBI/SBO, CB1.5-SBI/SBO, CB1.75-SBI/SBO, CB2-SBI/SBO, CB3-SBI/SBO, CB5-SBI/SBO, CB6-SBI/SBO, CB7-SBI/SBO, CB8-SBI/SBO, CB9-SBI/SBO, CB10;
C0.-SH, C0.3-SH, C0.4-SH, C0.5-SH, C0.6-SH, C0.7-SH, C0.9-SH, C1-SH, C1.25-SH, C1.75-SH, C2-SH, C3-SH, C4-SH, C5-SH, C6-SH, C7-SH, C9-SH, C10-SH;
PI0.1-SH, PI0.3-SH, PI0.4-SH, PI0.5-SH, PI0.6-SH, PI0.7-SH, PI0.9-SH, PI1-SH, PI1.25-SH, PI1.75-SH, PI2-SH, PI3-SH, PI4-SH, PIS-SH PI6-SH, PI7-SH, PI8-SH, PI9-SH, PI10-SH;
BK0.1-SH, BK0.3-SH, BK0.4-SH, BK0.5-SH, BK0.6-SH, BK0.7-SH, BK0.9-SH, BK1-SH, BK1.25-SH, BK1.75-SH, BK2-SH, BK3-SH, BK4-SH, BK5-SH, BK6-SH, BK7-SH, BK9-SH, BK10-SH;
CB0.1-SH, CB0.2-SH, CB0.3-S-SH, CB0.4SH, CB0.5-SH, CB0.6-SH, CB0.7-SH, CB0.9-SH, CB1-SH, CB2-SH, CB3-SH, CB4-SH, CB5-SH 8, CB6-SH, CB7-SH, CB8-SH, CB9-SH CN10-SH;
C1-MC2.5-SBI/SBO, C3-MC2.5-SBI/SBO, C6-MC2.5-SBI/SBO, C7-MC2.5-SBI/SBO,C9-MC2.5-SBI/SBO, C10-MC2.5-SBI/SBO, C1-MC5-SBI/SBO, C3-MC5-SBI/SBO, C6-MC5-SBI/SBO, C7-MC5-SBI/SBO, C9-MC5-SBI/SBO, C10-MC5-SBI/SBO C1-MC10-SBI/SBO, C2-MC10-SBI/SBO, C3-MC10-SBI/SBO, C6-MC10-SBI/SBO, C7-MC10-SBI/SBO,C9-MC10-SBI/SBO, C10-MC10-SBI/SBO;
PI1-MC2.5-SBI/SBO, PI3-MC2.5-SBI/SBO, PI6-MC2.5-SBI/SBO, PI7-MC2.5-SBI/SBO, PI9-MC2.5-SBI/SBO, PI1-MC5-SBI/SBO, PI3-MC5-SBI/SBO, PI6-MC5-SBI/SBO, PI7-MC5-SBI/SBO, PI9-MC5-SBI/SBO, PI 10-MC5-SBI/SBO, PI1-MC10-SBI/SBO, PI2-MC10-SBI/SBO, PI3-MC10-SBI/SBO, PI5-MC10-SBI/SBO, PI7-MC10-SBI/SBO,PI9-MC10-SBI/SBO, PI10-MC10-SBI/SBO;
BK1-MC2.5-SBI/SBO, BK3-MC2.5-SBI/SBO, BK6-MC2.5-SBI/SBO, BK7-MC2.5-SBI/SBO, BK9-MC2.5-SBI/SBO, BK10-MC2.5-SBI/SBO, BK1-MC5-SBI/SBO, BK3-MC5-SBI/SBO, BK6-MC5-SBI/SBO, BK7-MC5-SBI/SBO, BK9-MC5-SBI/SBO, BK10-MC5-SBI/SBO, BK1-MC10-SBI/SBO, BK2-MC10-SBI/SBO, BK3-MC10-SBI/SBO, BK5-MC10-SBI/SBO, BK7-MC10-SBI/SBO, BK9-MC10-SBI/SBO;
CB1-MC2.5-SBI/SBO, CB3-MC2.5-SBI/SBO, CB6-MC2.5-SBI/SBO, CB7-MC2.5-SBI/SBO, CB9-MC2.5-SBI/SBO, CB10-MC2.5-SBI/SBO, CB1-MC5-SBI/SBO, CB3-MC5-SBI/SBO, CB6-MC5-SBI/SBO, CB7-MC5-SBI/SBO, CB9-MC5-SBI/SBO, CB10-MC5-SBI/SBO, CB1-MC10-SBI/SBO, CB2-MC10-SBI/SBO, CB3-MC10-SBI/SBO, CB5-MC10-SBI/SBO, CB7-MC10-SBI/SBO,CB9-MC10-SBI/SBO, CB10-MC10-SBI/SBO;
C1-MC2.5-SH, C3-MC2.5-SH, C6-MC2.5-SH, C7-MC2.5-SH,C9-MC5-SH, C10-MC2.5-SH, C1-MC5-SH, C3-MC5-SH, C6-MC5-SH, C7-MC5-SH, C9-MC5-SH, C10-MC5-SH, C1-MC10-SH, C2-MC10-SH, C3-MC10-SH, C5-MC10-SH, C7-MC10-SH, C9-MC10-SH, C10-MC10-SH;
PI1-MC2.5-SH, PI3-MC2.5-SH, PI6-MC2.5-SH, PI7-MC2.5-SH, PI9-MC2.5-SH, PI10-MC2.5-SH, PI1-MC5-SH, PI3-MC5-SH, PI6-MC5-SH, PI7-MC5-SH, PI9-MC5-SH, PI10-MC5-SH, PI1-MC10-SH, PI2-MC10-SH, PI3-MC10-SH, PI5-MC10-SH, PI7-MC10-SH,PI9-MC10-SH, PI10-MC10-SH;
BK1-MC2.5-SH, BK3-MC2.5-SH, BK6-MC2.5-SH, BK7-MC2.5-SH, BK9-MC2.5-SH, BK10-MC2.5-SH, BK1-MC5-SH, BK3-MC5-SH, BK6-MC5-SH, BK7-MC5-SH, BK9-MC5-SH, BK10-MC5-SH, BK1-MC10-SH, BK2-MC10-SH, BK3-MC10-SH, BK5-MC10-SH, BK7-MC10-SH, BK9-MC10-SH, BK10-MC10-SH;
CB1-MC2.5-SH, CB3-MC2.5-SH, CB6-MC2.5-SH, CB7-MC2.5-SH, CB9-MC2.5-SH, CB10-MC2.5-SH, CB1-MC5-SH, CB3-MC5-SH, CB6-MC5-SH, CB7-MC5-SH, CB9-MC5-SH, CB10-MC5-SH, CB1-MC10-SH, CB2-MC10-SH, CB3-MC10-SH, CB5-MC10-SH, CB7-MC10-SH, CB9-MC10-SH, CB10-MC10-SH;
C1-P2.5-SBI/SBO, C3-P2.5-SBI/SBO, C6-P2.5-SBI/SBO, C7-P2.5-SBI/SBO, C9-P2.5-SBI/SBO, C10-P2.5-SBI/SBO, C1-P5-SBI/SBO, C3-P5-SBI/SBO, C6-P5-SBI/SBO, C7-P5-SBI/SBO, C9-P5-SBI/SBO, C10-P5-SBI/SBO, C1-P10-SBI/SBO, C2-P10-SBI/SBO, C3-P10-SBI/SBO, C5-P10-SBI/SBO, C7-P10-SBI/SBO, C9-P10-SBI/SBO, C10-P10-SBI/SBO;
PI1-P2.5-SBI/SBO, PI3-P2.5-SBI/SBO, PI6-P2.5-SBI/SBO, PI7-P2.5-SBI/SBO, PI9-P2.5-SBI/SBO, PI10-P2.5-SBI/SBO, PI1-P5-SBI/SBO, PI3-P5-SBI/SBO, PI6-P5-SBI/SBO, PI7-P5-SBI/SBO, PI9-P5-SBI/SBO, PI10-P5-SBI/SBO, PI1-P10-SBI/SBO, PI2-P10-SBI/SBO, PI3-P10-SBI/SBO, PI5-P10-SBI/SBO, PI7-P10-SBI/SBO, PI9-P10-SBI/SBO, PI10-P10-SBI/SBO;
BK1-P2.5-SBI/SBO, BK3-P2.5-SBI/SBO, BK6-P2.5-SBI/SBO, BK7-P2.5-SBI/SBO, BK9-P2.5-SBI/SBO, BK10-P2.5-SBI/SBO, BK1-P5-SBI/SBO, BK3-P5-SBI/SBO, BK6-P5-SBI/SBO, BK7-P5-SBI/SBO, BK9-P5-SBI/SBO, BK10-P5-SBI/SBO, BK1-P10-SBI/SBO, BK2-P10-SBI/SBO, BK3-P10-SBI/SBO, BK5-P10-SBI/SBO, BK7-P10-SBI/SBO, BK9-P10-SBI/SBO, BK10-P10-SBI/SBO;
CB1-P2.5-SBI/SBO, CB3-P2.5-SBI/SBO, CB6-P2.5-SBI/SBO, CB7-P2.5-SBI/SBO, CB9-P2.5-SBI/SBO, CB10-P2.5-SBI/SBO, CB1-P5-SBI/SBO, CB3-P5-SBI/SBO, CB6-P5-SBI/SBO, CB7-P5-SBI/SBO, CB9-P5-SBI/SBO, CB10-P5-SBI/SBO, CB1-P10-SBI/SBO, CB2-P10-SBI/SBO, CB3-P10-SBI/SBO, CB5-P10-SBI/SBO, CB7-P10-SBI/SBO, CB9-P10-SBI/SBO, CB10-P10-SBI/SBO;
C1-P2.5-SH, C3-P2.5-SH, C6-P2.5-SH, C7-P2.5-SH, C9-P2.5-SH, C10-P2.5-SH, C1-P5-SH, C3-P5-SH, C6-P5-SH, C7-P5-SH, C9-P5-SH, C10-P5-SH, C1-P10-SH, C2-P10-SH, C3-P10-SH, C5-P10-SH, C7-P10-SH, C9-P10-SH, C10-P10-SH;
PI1-P2.5-SH, PI3-P2.5-SH, PI6-P2.5-SH, PI7-P2.5-SH, PI9-P2.5-SH, PI10-P2.5-SH, PI1-P5-SH, PI3-P5-SH, PI6-P5-SH, PI7-P5-SH, PI9-P5-SH, PI10-P5-SH, PI1-P10-SH, PI2-P10-SH, PI3-P10-SH, PI5-P10-SH, PI7-P10-SH, PI9-P10-SH, PI10-P10-SH;
BK1-P2.5-SH, BK3-P2.5-SH, BK6-P2.5-SH, BK7-P2.5-SH, BK9-P2.5-SH, BK10-P2.5-SH, BK1-P5-SH, BK3-P5-SH, BK6-P5-SH, BK7-P5-SH, BK9-P5-SH, BK10-P5-SH, BK1-P10-SH, BK2-P10-SH, BK3-P10-SH, BK5-P10-SH, BK7-P10-SH, BK9-P10-SH, BK10-P10-SH;
CB1-P2.5-SH, CB3-P2.5-SH, CB6-P2.5-SH, CB7-P2.5-SH, CB9-P2.5-SH, CB10-P2.5-SH, CB1-P5-SH, CB3-P5-SH, CB6-P5-SH, CB7-P5-SH, CB9-P5-SH, CB10-P5-SH, CB1-P10-SH, CB2-P10-SH, CB3-P10-SH, CB5-P10-SH, CB7-P10-SH, CB9-P10-SH, CB10-P10-SH;

### Example 127

To a 100 ml beaker is added 40 ml of sterile water, 10 ml of 2% Chlorhexidine and 50 ml of 5% hydoxypropyl cellulose gel, which is vigorously stirred with an overhead stirrer. Sodium hydroxide of sufficient quantity is slowly added while stirring until dissolved to provide a pH in the range of 6.5-7.5. The viscosity of the solution is between 10,000-50,000 cps at 25° C. The solution remains stable for over 48 hours. The sample obtained is C0.2-HPC2.5-SH.

In a similar manner, the following samples are prepared using the components indicated:
C0.1-HPC2.5-SBI/SBO, C0.3-HPC2.5-SBI/SBO, C0.5-HPC2.5-SBI/SBO, C0.6-HPC2.5-SBI/SBO, C0.9-HPC2.5-SBI/SBO, C1-HPC2.5-SBI/SBO, C2-HPC2.5-SBI/SBO, C3-HPC2.5-SBI/SBO, C4-HPC2.5-SBI/SBO, C5-HPC2.5-SBI/SBO, C6-HPC2.5-SBI/SBO, C7-HPC2.5-SBI/SBO, C8-HPC2.5-SBI/SBO, C9-HPC2.5 C10-HPC2.5-SBI/SBO, C1-HPC5-SBI/SBO, C2-HPC5-SBI/SBO, C3-HPC5-SBI/SBO, C4-HPC5SBI/SBO, C7-HPC5-SBI/SBO, C9-HPC5-SBI/SBO, C10-HPC5-SBI/SBO, C1-HPC10-SBI/SBO, C2-HPC10-SBI/SBO, C3-HPC10-SBI/SBO, C4-HPC10-SBI/SBO, C5-HPC10-SBI/SBO, C6-HPC10-SBI/SBO, C7-HPC10-SBI/SBO, C8-HPC10-SBI/SBO, C9-HPC10-SBI/SBO, C10-HPC10-SBI/SBO;
PI1-HPC2.5-SBI/SBO, PI3-HPC2.5-SBI/SBO, PI6-HPC2.5-SBI/SBO, PI7-HPC2.5-SBI/SBO, PI9-HPC2.5-SBI/SBO, PI10-HPC2.5-SBI/SBO, PI1-HPC5-SBI/SBO, PI3-HPC5-SBI/SBO, PI6-HPC5-SBI/SBO, PI7-HPC5-SBI/SBO, PI9-HPC5-SBI/SBO, PI10-HPC5-SBI/SBO, PI1-HPC10-SBI/SBO, PI2-HPC10-SBI/SBO, PI3-HPC10-SBI/SBO, PI5-HPC10-SBI/SBO, PI7-HPC10-SBI/SBO, PI9-HPC10-SBI/SBO, PI10-HPC10-SBI/SBO;
BK1-HPC2.5-SBI/SBO, BK3-HPC2.5-SBI/SBO, BK6-HPC2.5-SBI/SBO, BK7-HPC2.5-SBI/SBO, BK9-HPC2.5-SBI/SBO, BK10-HPC2.5-SBI/SBO, BK1-HPC5-SBI/SBO, BK3-HPC5-SBI/SBO, BK6-HPC5-SBI/SBO, BK7-HPC5-SBI/SBO, BK9-HPC5-SBI/SBO, BK10-HPC5-SBI/SBO, BK1-HPC10-SBI/SBO, BK2-HPC10-SBI/SBO, BK3-HPC10-SBI/SBO, BK5-HPC10-SBI/SBO, BK7-HPC10-SBI/SBO, BK9-HPC10-SBI/SBO, BK10-HPC10-SBI/SBO;
CB1-HPC2.5-SBI/SBO, CB3-HPC2.5-SBI/SBO, CB6-HPC2.5-SBI/SBO, CB7-HPC2.5-SBI/SBO, CB9-HPC2.5-SBI/SBO, CB10-HPC2.5-SBI/SBO, CB1-HPC5-SBI/SBO, CB3-HPC5-SBI/SBO, CB6-HPC5-SBI/SBO, CB7-HPC5-SBI/SBO, CB9-HPC5-SBI/SBO, CB10-HPC5-SBI/SBO, CB1-HPC10-SBI/SBO, CB2-HPC10-SBI/SBO, CB3-HPC10-SBI/SBO, CB5-HPC10-SBI/SBO, CB7-HPC10-SBI/SBO, CB9-HPC10-SBI/SBO, CB10-HPC10-SBI/SBO;
C1-HPC2.5-SH, C3-HPC2.5-SH, C6-HPC2.5-SH, C7-HPC2.5-SH, C9-HPC2.5-SH, C10-HPC2.5-SH, C1-HPC5-SH, C3-HPC5-SH, C6-HPC5-SH, C7-HPC5-SH, C9-HPC5-SH, C10-HPC5-SH, C1-HPC10-SH, C2-HPC10-SH, C3-HPC10-SH, C5-HPC10-SH, C7-HPC10-SH, C9-HPC10-SH, C10-HPC10-SH;
PI1-HPC2.5-SH, PI3-HPC2.5-SH, PI6-HPC2.5-SH, PI7-HPC2.5-SH, PI9-HPC2.5-SH, PI10-HPC2.5-SH, PI1-HPC5-SH, PI3-HPC5-SH, PI6-HPC5-SH, PI7-HPC5-SH, PI9-HPC5-SH, PI10-HPC5-SH, PI1-HPC10-SH, PI2-HPC10-SH, PI3-HPC10-SH, PI5-HPC10-SH, PI7-HPC10-SH, PI9-HPC10-SH, PI10-HPC10-SH;
BK1-HPC2.5-SH, BK3-HPC2.5-SH, BK6-HPC2.5-SH, BK7-HPC2.5-SH, BK9-HPC2.5-SH, BK10-HPC2.5-SH, BK1-HPC5-SH, BK3-HPC5-SH, BK6-HPC5-SH, BK7-HPC5-SH, BK9-HPC5-SH, BK10-HPC5-SH, BK1-HPC10-SH, BK2-HPC10-SH, BK3-HPC10-SH, BK5-HPC10-SH, BK7-HPC10-SH, BK9-HPC10-SH, BK10-HPC10-SH;
CB1-HPC2.5-SH, CB3-HPC2.5-SH, CB6-HPC2.5-SH, CB7-HPC2.5-SH, CB9-HPC2.5-SH, CB10-HPC2.5-SH, CB1-HPC5-SH, CB3-HPC5-SH, CB6-HPC5-SH, CB7-HPC5-SH, CB9-HPC5-SH, CB10-HPC5-SH, CB1-HPC10-SH, CB2-HPC10-SH, CB3-HPC10-SH, CB5-HPC10-SH, CB7-HPC10-SH, CB9-HPC10-SH, CB10-HPC10-SH.

Example 128

To a 100 ml beaker is added 40 ml of sterile water, 10 ml of 2% Chlorhexidine and 50 ml of 5% methyl hydroxypropyl cellulose gel, which is vigorously stirred with an overhead stirrer. Sodium hydroxide of sufficient quantity is slowly added while stirring until dissolved to provide a pH in the range of 6.5-7.5. The viscosity of the solution is between 10,000-50,000 cps at 25° C. The solution remains stable for over 48 hours. The sample obtained is C0.2-MHPC2.5-SH.

In a similar manner, the following samples are prepared using the components indicated:
C1-MHPC2.5-SBI/SBO, C3-MHPC2.5-SBI/SBO, C6-MHPC2.5-SBI/SBO, C7-MHPC2.5-SBI/SBO, C9-MHPC2.5-SBI/SBO, C10-MHPC2.5-SBI/SBO, C1-MHPC5-SBI/SBO, C3-MHPC5-SBI/SBO, C6-MHPC5-SBI/SBO, C7-MHPC5-SBI/SBO, C9-MHPC5-SBI/SBO, C10-MHPC5-SBI/SBO, C1-MHPC10-SBI/SBO, C2-MHPC10-SBI/SBO, C3-MHPC10-SBI/SBO, C5-MHPC10-SBI/SBO, C7-MHPC10-SBI/SBO, C9-MHPC10-SBI/SBO, C10-MHPC10-SBI/SBO;
PI1-MHPC2.5-SBI/SBO, PI3-MHPC2.5-SBI/SBO, PI6-MHPC2.5-SBI/SBO, PI7-MHPC2.5-SBI/SBO, PI9-MHPC2.5-SBI/SBO, PI10-MHPC2.5-SBI/SBO, PI1-MHPC5-SBI/SBO, PI3-MHPC5-SBI/SBO, PI6-MHPC5-SBI/SBO, PI7-MHPC5-SBI/SBO, PI9-MHPC5-SBI/SBO, PI10-MHPC5-SBI/SBO, PI1-MHPC10-SBI/SBO, PI2-MHPC10-SBI/SBO, PI3-MHPC10-SBI/SBO, PI5-MHPC10-SBI/SBO, PI7-MHPC10-SBI/SBO, PI9-MHPC10-SBI/SBO, PI10-MHPC10-SBI/SBO;
BK1-MHPC2.5-SBI/SBO, BK3-MHPC2.5-SBI/SBO, BK6-MHPC2.5-SBI/SBO, BK7-MHPC2.5-SBI/SBO, BK9-MHPC2.5-SBI/SBO, BK10-MHPC2.5-SBI/SBO, BK1-MHPC5-SBI/SBO, BK3-MHPC5-SBI/SBO, BK6-MHPC5-SBI/SBO, BK7-MHPC5-SBI/SBO, BK9-MHPC5-SBI/SBO, BK10-MHPC5-SBI/SBO, BK1-MHPC10-SBI/SBO, BK2-MHPC10-SBI/SBO, BK3-MHPC10-SBI/SBO, BK5-MHPC10-SBI/SBO, BK7-MHPC10-SBI/SBO, BK9-MMHPC10-SBI/SBO, BK10-MMHPC10-SBI/SBO;
CB1-MHPC2.5-SBI/SBO, CB3-MHPC2.5-SBI/SBO, CB6-MHPC2.5-SBI/SBO, CB7-MHPC2.5-SBI/SBO, CB9-MHPC2.5-SBI/SBO, CB10-MHPC2.5-SBI/SBO, CB1-MHPC5-SBI/SBO, CB3-MHPC5-SBI/SBO, CB6-MHPC5-SBI/SBO, CB7-MHPC5-SBI/SBO, CB9-MHPC5-SBI/SBO, CB10-MHPC5-SBI/SBO, CB1-MHPC10-SBI/SBO, CB2-MHPC10-SBI/SBO, CB3-MHPC10-SBI/SBO, CB5-MHPC10-SBI/SBO, CB7-MHPC10-SBI/SBO, CB9-MHPC10-SBI/SBO, CB10-MHPC10-SBI/SBO;
C1-MHPC2.5-SH, C3-MHPC2.5-SH, C6-MHPC2.5-SH, C7-MHPC2.5-SH, C9-MHPC2.5-SH, C10-MHPC2.5-SH, C1-MHPC5-SH, C3-MHPC5-SH, C6-MHPC5-SH, C7-MHPC5-SH, C9-MHPC5-SH, C10-MHPC5-SH, C1-MHPC10-SH, C2-MHPC10-SH, C3-MHPC10-SH, C5-MHPC10-SH, C7-MHPC10-SH, C9-MHPC10-SH, C10-MHPC10-SH;
PI1-MHPC2.5-SH, PI3-MHPC2.5-SH, PI6-MHPC2.5-SH, PI7-MHPC2.5-SH, PI9-MHPC2.5-SH, PI10-MHPC2.5-SH, PI1-MHPC5-SH, PI3-MHPC5-SH, PI6-MHPC5-SH, PI7-MHPC5-SH, PI9-MHPC5-SH, PI10-MHPC5-SH, PI1-MHPC10-SH, PI2-MHPC10-SH, PI3-MHPC10-SH, PI5-MHPC10-SH, PI7-MHPC10-SH, PI9-MHPC10-SH, PI10-MHPC10-SH;
BK1-MHPC2.5-SH, BK3-MHPC2.5-SH, BK6-MHPC2.5-SH, BK7-MHPC2.5-SH, BK9-MHPC2.5-SH, BK10-MHPC2.5-SH, BK1-MHPC5-SH, BK3-MHPC5-SH, BK6-MHPC5-SH, BK7-MHPC5-SH, BK9-MHPC5-SH, BK10-MHPC5-SH, BK1-MHPC10-SH, BK2-MHPC10-SH, BK3-MHPC10-SH, BK5-MHPC10-SH, BK7-MHPC10-SH, BK9-MHPC10-SH, BK10-MHPC10-SH;
CB1-MHPC2.5-SH, CB3-MHPC2.5-SH, CB6-MHPC2.5-SH, CB7-MHPC2.5-SH, CB9-MHPC2.5-SH, CB10-MHPC2.5-SH, CB1-MHPC5-SH, CB3-MHPC5-SH, CB6-MHPC5-SH, CB7-MHPC5-SH, CB9-MHPC5-SH, CB10-MHPC5-SH, CB1-MHPC10-SH, CB2-MHPC10-SH, CB3-MHPC10-SH, CB5-MHPC10-SH, CB7-MHPC10-SH, CB9-MHPC10-SH, CB10-MHPC10-SH;

### Example 129

To a 100 ml beaker is added 40 ml of sterile water, 10 ml of 2% Chlorhexidine and 50 ml of 5% hydoxypropyl methyl cellulose gel, which is vigorously stirred with an overhead stirrer. Sodium hydroxide of sufficient quantity is slowly added while stirring until dissolved to provide a pH in the range of 6.5-7.5. The viscosity of the solution is between 10,000-50,000 cps at 25° C. The solution remains stable for over 48 hours. The sample obtained is C0.2-HPMC-SH.

In a similar manner, the following samples are prepared using the components indicated:
C1-HPMC2.5-SBI/SBO, C3-HPMC2.5-SBI/SBO, C6-HPMC2.5-SBI/SBO, C7-HPMC2.5-SBI/SBO, C9-HPMC2.5-SBI/SBO, C10-HPMC2.5-SBI/SBO, C1-HPMC5-SBI/SBO, C3-HPMC5-SBI/SBO, C6-HPMC5-SBI/SBO, C7-HPMC5-SBI/SBO, C9-HPMC5-SBI/SBO, C11-HPMC5-SBI/SBO, C12-HPMC5-SBI/SBO, C13-HPMC5-SBI/SBO, C14-HPMC5-SBI/SBO, C15-HPMC5-SBI/SBO, C16-HPMC5-SBI/SBO, C17-HPMC5-SBI/SBO, C18-HPMC5-SBI/SBO, C19-HPMC5 C20-HPMC5-SBI/SBO, C1-HPMC10-SBI/SBO, C2-HPMC10-SBI/SBO, C3-HPMC10-SBI/SBO, C5-HPMC10-SBI/SBO, C7-HPMC10-SBI/SBO, C9-HPMC10-SBI/SBO, C11-HPMC10-SBI/SBO, C12-HPMC10-SBI/SBO, C13-HPMC10-SBI/SBO, C14-HPMC10-SBI/SBO, C15-HPMC10-SBI/SBO, C16-HPMC10-SBI/SBO, C17-HPMC10-SBI/SBO, C18-HPMC10-SBI/SBO, C19-HPMC10-SBI/SBO, C20-HPMC10-SBI/SBO,
PI1-HPMC2.5-SBI/SBO, PI3-HPMC2.5-SBI/SBO, PI6-HPMC2.5-SBI/SBO, PI7-HPMC2.5-SBI/SBO, PI9-HPMC2.5-SBI/SBO, PI10-HPMC2.5-SBI/SBO, PI1-HPMC5-SBI/SBO, PI3-HPMC5-SBI/SBO, PI6-HPMC5-SBI/SBO, PI7-HPMC5-SBI/SBO, PI9-HPMC5-SBI/SBO, PI11-HPMC5-SBI/SBO, PI12-HPMC5-SBI/SBO, PI13-HPMC5-SBI/SBO, PI14-HPMC5-SBI/SBO, PI15-HPMC5-SBI/SBO, PI16-HPMC5-SBI/SBO, PI17-HPMC5-SBI/SBO, PI18-HPMC5-SBI/SBO, PI19-HPMC5 PI20-HPMC5-SBI/SBO, PI1-HPMC10-SBI/SBO, PI2-HPMC10-SBI/SBO, PI3-HPMC10-SBI/SBO, PI5-HPMC10-SBI/SBO, PI7-HPMC10-SBI/SBO, PI9-HPMC10-SBI/SBO, PI11-HPMC10-SBI/SBO, PI12-HPMC10-SBI/SBO, PI13-HPMC10-SBI/SBO, PI14-HPMC10-SBI/SBO, PI15-HPMC10-SBI/SBO, PI16-HPMC10-SBI/SBO, PI17-HPMC10-SBI/SBO, PI18-HPMC10-SBI/SBO, PI19-HPMC10-SBI/SBO, PI20-HPMC10-SBI/SBO,
BK1-HPMC2.5-SBI/SBO, BK3-HPMC2.5-SBI/SBO, BK6-HPMC2.5-SBI/SBO, BK7-HPMC2.5-SBI/SBO, BK9-HPMC2.5-SBI/SBO, BK10-HPMC2.5-SBI/SBO, BK1-HPMC5-SBI/SBO, BK3-HPMC5-SBI/SBO, BK6-HPMC5-SBI/SBO, BK7-HPMC5-SBI/SBO, BK9-HPMC5-SBI/SBO, BK11-HPMC5-SBI/SBO, BK12-HPMC5-SBI/SBO, BK13-HPMC5-SBI/SBO, BK14-HPMC5-SBI/SBO, BK15-HPMC5-SBI/SBO, BK16-HPMC5-SBI/SBO, BK17-HPMC5-SBI/SBO, BK18-HPMC5-SBI/SBO, BK19-HPMC5 BK20-HPMC5-SBI/SBO, BK1-HPMC10-SBI/SBO, BK2-HPMC10-SBI/SBO, BK3-HPMC10-SBI/SBO, BK5-HPMC10-SBI/SBO, BK7-HPMC10-SBI/SBO, BK9-MHPMC10-SBI/SBO, BK11-MHPMC10-SBI/SBO, BK12-MHPMC10-SBI/SBO, BK13-MHPMC10-SBI/SBO, BK14-MHPMC10-SBI/SBO, BK15-HPMC10-SBI/SBO, BK16-HPMC10-SBI/SBO, BK17-HPMC10-SBI/SBO, BK18-HPMC10-SBI/SBO, BK19-HPMC10-SBI/SBO, BK20-HPMC10-SBI/SBO,
CB1-HPMC2.5-SBI/SBO, CB3-HPMC2.5-SBI/SBO, CB6-HPMC2.5-SBI/SBO, CB7-HPMC2.5-SBI/SBO, CB9-HPMC2.5-SBI/SBO, CB10-HPMC2.5-SBI/SBO, CB1-HPMC5-SBI/SBO, CB3-HPMC5-SBI/SBO, CB6-HPMC5-SBI/SBO, CB7-HPMC5-SBI/SBO, CB9-HPMC5-SBI/SBO, CB11-HPMC5-SBI/SBO, CB12-HPMC5-SBI/SBO, CB13-HPMC5-SBI/SBO, CB14-HPMC5-SBI/SBO, CB15-HPMC5-SBI/SBO, CB 16-HPMC5-SBI/SBO, CB17-HPMC5-SBI/SBO, CB18-HPMC5-SBI/SBO, CB19-HPMC5 CB20-HPMC5-SBI/SBO, CB1-HPMC10-SBI/SBO, CB2-HPMC10-SBI/SBO, CB3-HPMC10-SBI/SBO, CB5-HPMC10-SBI/SBO, CB7-HPMC10-SBI/SBO, CB9-HPMC10-SBI/SBO, CB11-HPMC10-SBI/SBO, CB12-HPMC10-SBI/SBO, CB13-HPMC10-SBI/SBO, CB14-HPMC10-SBI/SBO, CB15-HPMC10-SBI/SBO, CB16-HPMC10-SBI/SBO, CB17-HPMC10-SBI/SBO, CB18-HPMC10-SBI/SBO, CB19-HPMC10-SBI/SBO, CB20-HPMC10-SBI/SBO,
C1-HPMC2.5-SH, C3-HPMC2.5-SH, C6-HPMC2.5-SH, C7-HPMC2.5-SH, C9-HPMC2.5-SH, C10-HPMC2.5-SH, C1-HPMC5-SH, C3-HPMC5-SH, C6-HPMC5-SH, C7-HPMC5-SH, C9-HPMC5-SH, C11-HPMC5-SH, C12-HPMC5-SH, C13-HPMC5-SH, C14-HPMC5-SH, C15-HPMC5-SH, C16-HPMC5-SH, C17-HPMC5-SH, C18-HPMC5-SH, C19-HPMC5-SH, C20-HPMC5-SH, C1-HPMC10-SH, C2-HPMC10-SH, C3-HPMC10-SH, C5-HPMC10-SH, C7-HPMC10-SH, C9-HPMC10-SH, C11-HPMC10-SH, C12-HPMC10-SH, C13-HPMC10-SH, C14-HPMC10-SH, C15-HPMC10-SH, C16-HPMC10-SH, C17-HPMC10-SH, C18-HPMC10-SH, C19-HPMC10-SH, C20-HPMC10-SH,
PI1-HPMC2.5-SH, PI3-HPMC2.5-SH, PI6-HPMC2.5-SH, PI7-HPMC2.5-SH, PI9-HPMC2.5-SH, PI10-HPMC2.5-SH, PI1-HPMC5-SH, PI3-HPMC5-SH, PI6-HPMC5-SH, PI7-HPMC5-SH, PI9-HPMC5-SH, PI11-HPMC5-SH, PI12-HPMC5-SH, PI13-HPMC5-SH, PI14-HPMC5-SH, PI15-HPMC5-SH, PI16-HPMC5-SH, PI17-HPMC5-SH, PI18-HPMC5-SH, PI19-HPMC5 PI20-HPMC5-SH, PI1-HPMC10-SH, PI2-HPMC10-SH, PI3-HPMC10-SH, PI5-HPMC10-SH, PI7-HPMC10-SH, PI9-HPMC10-SH, PI11-HPMC10-SH, PI12-HPMC10-SH, PI13-HPMC10-SH, PI14-HPMC10-SH, PI15-HPMC10-SH, PI16-HPMC10-SH, PI17-HPMC10-SH, PI18-HPMC10-SH, PI19-HPMC10-SH, PI20-HPMC10-SH,
BK1-HPMC2.5-SH, BK3-HPMC2.5-SH, BK6-HPMC2.5-SH, BK7-HPMC2.5-SH, BK9-HPMC2.5-SH, BK10-HPMC2.5-SH, BK1-HPMC5-SH, BK3-HPMC5-SH, BK6-HPMC5-SH, BK7-HPMC5-SH, BK9-HPMC5-SH, BK11-HPMC5-SH, BK12-HPMC5-SH, BK13-HPMC5-SH, BK14-HPMC5-SH, BK15-HPMC5-SH, BK16-HPMC5-SH, BK17-HPMC5-SH, BK18-HPMC5-SH, BK19-HPMC5 BK20-HPMC5-SH, BK1-HPMC10-SH, BK2-HPMC10-SH, BK3-HPMC10-SH, BK5-HPMC10-SH, BK7-HPMC10-SH, BK9-HPMC10-SH, BK11-HPMC10-SH, BK12-HPMC10-SH, BK13-HPMC10-SH, BK14-HPMC10-SH, BK15-HPMC10-SH, BK16-HPMC10-SH, BK17-HPMC10-SH, BK18-HPMC10-SH, BK19-HPMC10-SH, BK20-HPMC10-SH,
CB1-HPMC2.5-SH, CB3-HPMC2.5-SH, CB6-HPMC2.5-SH, CB7-HPMC2.5-SH, CB9-HPMC2.5-SH, CB10-HPMC2.5-SH, CB1-HPMC5-SH, CB3-HPMC5-SH, CB6-HPMC5-SH, CB7-HPMC5-SH, CB9-HPMC5-SH, CB11-HPMC5-SH, CB12-HPMC5-SH, CB13-HPMC5-SH, CB14-HPMC5-SH, CB15-HPMC5-SH, CB16-HPMC5-SH, CB17-HPMC5-SH, CB18-HPMC5-SH, CB19-HPMC5 CB20-HPMC5-SH, CB1-HPMC10-SH, CB2-HPMC10-SH, CB3-HPMC10-SH, CB5-HPMC10-SH, CB7-HPMC10-SH, CB9-HPMC10-SH, CB11-HPMC10-SH, CB12-HPMC10-SH, CB13-HPMC10-SH, CB14-HPMC10-SH, CB15-HPMC10-SH, CB16-HPMC10-SH, CB17-HPMC10-SH, CB18-HPMC10-SH, CB19-HPMC10-SH, CB20-HPMC10-SH,

### Example 130

To a 100 ml beaker is added 40 ml of sterile water, 10 ml of 2% Chlorhexidine and 50 ml of 5% cellulose acetate gel, which is vigorously stirred with an overhead stirrer. Sodium hydroxide of sufficient quantity is slowly added while stirring until dissolved to provide a pH in the range of 6.5-7.5. The viscosity of the solution is between 10,000-50,000 cps at 25° C. The solution remains stable for over 48 hours. The sample obtained is C0.2-CA2.5-SH,

In a similar manner, the following samples are prepared using the components indicated:
C1-CA5-SBI/SBO, C3-CA5-SBI/SBO, C6-CA5-SBI/SBO, C7-CA5-SBI/SBO, C9-CA5-SBI/SBO, C11-CA5-SBI/SBO, C12-CA5-SBI/SBO, C13-CA5-SBI/SBO, C14-CA5-SBI/SBO, C15-CA5-SBI/SBO, C16-CA5-SBI/SBO, C17-CA5-SBI/SBO, C18-CA5-SBI/SBO, C19-CA5 C20-CA5-SBI/SBO, C1-CA10-SBI/SBO, C2-CA10-SBI/SBO, C3-CA10-SBI/SBO, C5-CA10-SBI/SBO, C7-CA10-SBI/SBO, C9-CA10-SBI/SBO, C11-CA10-SBI/SBO, C12-CA10-SBI/SBO, C13-CA10-SBI/SBO, C14-CA10-SBI/SBO, C15-CA10-SBI/SBO, C16-CA10-SBI/SBO, C17-CA10-SBI/SBO, C18-CA10-SBI/SBO, C19-CA10-SBI/SBO, C20-CA10-SBI/SBO,
PI1-CA5-SBI/SBO, PI3-CA5-SBI/SBO, PI6-CA5-SBI/SBO, PI7-CA5-SBI/SBO, PI9-CA5-SBI/SBO, PI11-CA5-SBI/SBO, PI12-CA5-SBI/SBO, PI13-CA5-SBI/SBO, PI14-CA5-SBI/SBO, PI15-CA5-SBI/SBO, PI16-CA5-SBI/SBO, PI17-CA5-SBI/SBO, PI18-CA5-SBI/SBO, PI19-CA5 PI20-CA5-SBI/SBO, PI1-CA10-SBI/SBO, PI2-CA10-SBI/SBO, PI3-CA10-SBI/SBO, PI5-CA10-SBI/SBO, PI7-CA10-SBI/SBO, PI9-CA10-SBI/SBO, PI11-CA10-SBI/SBO, PI12-CA10-SBI/SBO, PI13-CA10-SBI/SBO, PI14-CA10-SBI/SBO, PI15-CA10-SBI/SBO, PI16-CA10-SBI/SBO, PI17-CA10-SBI/SBO, PI18-CA10-SBI/SBO, PI19-CA10-SBI/SBO, PI20-CA10-SBI/SBO,
BK1-CA5-SBI/SBO, BK3-CA5-SBI/SBO, BK6-CA5-SBI/SBO, BK7-CA5-SBI/SBO, BK9-CA5-SBI/SBO, BK11-CA5-SBI/SBO, BK12-CA5-SBI/SBO, BK13-CA5-SBI/SBO, BK14-CA5-SBI/SBO, BK15-CA5-SBI/SBO, BK16-CA5-SBI/SBO, BK17-CA5-SBI/SBO, BK18-CA5-SBI/SBO, BK19-CA5 BK20-CA5-SBI/SBO, BK1-CA10-SBI/SBO, BK2-CA10-SBI/SBO, BK3-CA10-SBI/SBO, BK5-CA10-SBI/SBO, BK7-CA10-SBI/SBO, BK9-CA10-SBI/SBO, BK11-CA10-SBI/SBO, BK12-CA10-SBI/SBO, BK13-CA10-SBI/SBO, BK14-CA10-SBI/SBO, BK15-CA10-SBI/SBO, BK16-CA10-SBI/SBO, BK17-CA10-SBI/SBO, BK18-CA10-SBI/SBO, BK19-CA10-SBI/SBO, BK20-CA10-SBI/SBO,
CB1-CA5-SBI/SBO, CB3-CA5-SBI/SBO, CB6-CA5-SBI/SBO, CB7-CA5-SBI/SBO, CB9-CA5-SBI/SBO, CB11-CA5-SBI/SBO, CB12-CA5-SBI/SBO, CB13-CA5-SBI/SBO, CB14-CA5-SBI/SBO, CB15-CA5-SBI/SBO, CB16-CA5-SBI/SBO, CB17-CA5-SBI/SBO, CB18-CA5-SBI/SBO, CB19-CA5 CB20-CA5-SBI/SBO, CB1-CA10-SBI/SBO, CB2-CA10-SBI/SBO, CB3-CA10-SBI/SBO, CB5-CA10-SBI/SBO, CB7-CA10-SBI/SBO, CB9-CA10-SBI/SBO, CB11-CA10-SBI/SBO, CB12-CA10-SBI/SBO, CB13-CA10-SBI/SBO, CB14-CA10-SBI/SBO, CB15-CA10-SBI/SBO, CB16-CA10-SBI/SBO, CB17-CA10-SBI/SBO, CB18-CA10-SBI/SBO, CB19-CA10-SBI/SBO, CB20-CA10-SBI/SBO,
C1-CA5-SH, C3-CA5-SH, C6-CA5-SH, C7-CA5-SH, C9-CA5-SH, C11-CA5-SH, C12-CA5-SH, C13-CA5-SH, C14-CA5-SH, C15-CA5-SH, C16-CA5-SH, C17-CA5-SH, C18-CA5-SH, C19-CA5-SH, C20-CA5-SH, C1-CA10-SH, C2-CA10-SH, C3-CA10-SH, C5-CA10-SH, C7-CA10-SH, C9-CA10-SH, C11-CA10-SH, C12-CA10-SH, C13-CA10-SH, C14-CA10-SH, C15-CA10-SH, C16-CA10-SH, C17-CA10-SH, C18-CA10-SH, C19-CA10-SH, C20-CA10-SH,
PI1-CA5-SH, PI3-CA5-SH, PI6-CA5-SH, PI7-CA5-SH, PI9-CA5-SH, PI11-CA5-SH, PI12-CA5-SH, PI13-CA5-SH, PI14-CA5-SH, PI15-CA5-SH, PI16-CA5-SH, PI17-CA5-SH, PI18-CA5-SH, PI19-CA5 PI20-CA5-SH, PI1-CA10-SH, PI2-CA10-SH, PI3-CA10-SH, PI5-CA10-SH, PI7-CA10-SH, PI9-CA10-SH, PI11-CA10-SH, PI12-CA10-SH, PI13-CA10-SH, PI14-CA10-SH, PI15-CA10-SH, PI16-CA10-SH, PI17-CA10-SH, PI18-CA10-SH, PI19-CA10-SH, PI20-CA10-SH,
BK1-CA5-SH, BK3-CA5-SH, BK6-CA5-SH, BK7-CA5-SH, BK9-CA5-SH, BK11-CA5-SH, BK12-CA5-SH, BK13-CA5-SH, BK14-CA5-SH, BK15-CA5-SH, BK16-CA5-SH, BK17-CA5-SH, BK18-CA5-SH, BK19-CA5 BK20-CA5-SH, BK1-CA10-SH, BK2-CA10-SH, BK3-CA10-SH, BK5-CA10-SH, BK7-CA10-SH, BK9-CA10-SH, BK11-CA10-SH, BK12-CA10-SH, BK13-CA10-SH, BK14-CA10-SH, BK15-CA10-SH, BK16-CA10-SH, BK17-CA10-SH, BK18-CA10-SH, BK19-CA10-SH, BK20-CA10-SH,
CB1-CA5-SH, CB3-CA5-SH, CB6-CA5-SH, CB7-CA5-SH, CB9-CA5-SH, CB11-CA5-SH, CB12-CA5-SH, CB13-CA5-SH, CB14-CA5-SH, CB15-CA5-SH, CB16-CA5-SH, CB17-CA5-SH, CB18-CA5-SH, CB19-CA5 CB20-CA5-SH, CB1-CA10-SH, CB2-CA10-SH, CB3-CA10-SH, CB5-CA10-SH, CB7-CA10-SH, CB9-CA10-SH, CB11-CA10-SH, CB12-CA10-SH, CB13-CA10-SH, CB14-CA10-SH, CB15-CA10-SH, CB16-CA10-SH, CB17-CA10-SH, CB18-CA10-SH, CB19-CA10-SH, CB20-CA10-SH,

### Example 131

To a 100 ml beaker is added 40 ml of sterile water, 10 ml of 2% Chlorhexidine and 50 ml of 5% ethyl cellulose gel, which is vigorously stirred with an overhead stirrer. Sodium hydroxide of sufficient quantity is slowly added while stirring until dissolved to provide a pH in the range of 6.5-7.5. The viscosity of the solution is between 10,000-50,000 cps at 25° C. The solution remains stable for over 48 hours. The sample obtained is C0.2-EC2.5-SH,

In a similar manner, the following samples are prepared using the components indicated:
C1-EC5-SBI/SBO, C3-EC5-SBI/SBO, C6-EC5-SBI/SBO, C7-EC5-SBI/SBO, C9-EC5-SBI/SBO, C11-EC5-SBI/SBO, C12-EC5-SBI/SBO, C13-EC5-SBI/SBO, C14-EC5-SBI/SBO, C15-EC5-SBI/SBO, C16-EC5-SBI/SBO, C17-EC5-SBI/SBO, C18-EC5-SBI/SBO, C19-EC5 C20-EC5-SBI/SBO, C1-EC10-SBI/SBO, C2-EC10-SBI/SBO, C3-EC10-SBI/SBO, C5-EC10-SBI/SBO, C7-EC10-SBI/SBO, C9-EC10-SBI/SBO, C11-EC10-SBI/SBO, C12-EC10-SBI/SBO, C13-EC10-SBI/SBO, C14-EC10-SBI/SBO, C15-EC10-SBI/SBO, C16-EC10-SBI/SBO, C17-EC10-SBI/SBO, C18-EC10-SBI/SBO, C19-EC10-SBI/SBO, C20-EC10-SBI/SBO,
PI1-EC5-SBI/SBO, PI3-EC5-SBI/SBO, PI6-EC5-SBI/SBO, PI7-EC5-SBI/SBO, PI9-EC5-SBI/SBO, PI11-EC5-SBI/SBO, PI12-EC5-SBI/SBO, PI13-EC5-SBI/SBO, PI14-EC5-SBI/SBO, PI15-EC5-SBI/SBO, PI16-EC5-SBI/SBO, PI17-EC5-SBI/SBO, PI18-EC5-SBI/SBO, PI19-EC5 PI20-EC5-SBI/SBO, PI1-EC10-SBI/SBO, PI2-EC10-SBI/SBO, PI3-EC10-SBI/SBO, PI5-EC10-SBI/SBO, PI7-EC10-SBI/SBO, PI9-EC10-SBI/SBO, PI11-EC10-SBI/SBO, PI12-EC10-SBI/SBO, PI13-EC10-SBI/SBO, PI14-EC10-SBI/SBO, PI15-EC10-SBI/SBO, PI16-EC10-SBI/SBO, PI17-EC10-SBI/SBO, PI18-EC10-SBI/SBO, PI19-EC10-SBI/SBO, PI20-EC10-SBI/SBO,
BK1-EC5-SBI/SBO, BK3-EC5-SBI/SBO, BK6-EC5-SBI/SBO, BK7-EC5-SBI/SBO, BK9-EC5-SBI/SBO, BK11-EC5-SBI/SBO, BK12-EC5-SBI/SBO, BK13-EC5-SBI/SBO, BK14-EC5-SBI/SBO, BK15-EC5-SBI/SBO, BK16-EC5-SBI/SBO, BK17-EC5-SBI/SBO, BK18-EC5-SBI/SBO, BK19-EC5 BK20-EC5-SBI/SBO, BK1-EC10-SBI/SBO, BK2-EC10-SBI/SBO, BK3-EC10-SBI/SBO, BK5-EC10-SBI/SBO, BK7-EC10-SBI/SBO, BK9-EC10-SBI/SBO, BK11-EC10-SBI/SBO, BK12-EC10-SBI/SBO, BK13-EC10-SBI/SBO, BK14-EC10-SBI/SBO, BK15-EC10-SBI/SBO, BK16-EC10-SBI/SBO, BK17-EC10-SBI/SBO, BK18-EC10-SBI/SBO, BK19-EC10-SBI/SBO, BK20-EC10-SBI/SBO,
CB1-EC5-SBI/SBO, CB3-EC5-SBI/SBO, CB6-EC5-SBI/SBO, CB7-EC5-SBI/SBO, CB9-EC5-SBI/SBO, CB11-EC5-SBI/SBO, CB12-EC5-SBI/SBO, CB13-EC5-SBI/SBO, CB14-EC5-SBI/SBO, CB15-EC5-SBI/SBO, CB16-EC5-SBI/SBO, CB17-EC5-SBI/SBO, CB18-EC5-SBI/SBO, CB19-EC5 CB20-EC5-SBI/SBO, CB1-EC10-SBI/SBO, CB2-EC10-SBI/SBO, CB3-EC10-SBI/SBO, CB5-EC10-SBI/SBO, CB7-EC10-SBI/SBO, CB9-EC10-SBI/SBO, CB11-EC10-SBI/SBO, CB12-EC10-SBI/SBO, CB13-EC10-SBI/SBO, CB14-EC10-SBI/SBO, CB15-EC10-SBI/SBO, CB16-EC10-SBI/SBO, CB17-EC10-SBI/SBO, CB18-EC10-SBI/SBO, CB19-EC10-SBI/SBO, CB20-EC10-SBI/SBO,
C1-EC5-SH, C3-EC5-SH, C6-EC5-SH, C7-EC5-SH, C9-EC5-SH, C11-EC5-SH, C12-EC5-SH, C13-EC5-SH, C14-EC5-SH, C15-EC5-SH, C16-EC5-SH, C17-EC5-SH, C18-EC5-SH, C19-EC5-SH, C20-EC5-SH, C1-EC10-SH, C2-EC10-SH, C3-EC10-SH, C5-EC10-SH, C7-EC10-SH, C9-EC10-SH, C11-EC10-SH, C12-EC10-SH, C13-EC10-SH, C14-EC10-SH, C15-EC10-SH, C16-EC10-SH, C17-EC10-SH, C18-EC10-SH, C19-EC10-SH, C20-EC10-SH,
PI1-EC5-SH, PI3-EC5-SH, PI6-EC5-SH, PI7-EC5-SH, PI9-EC5-SH, PI11-EC5-SH, PI12-EC5-SH, PI13-EC5-SH, PI14-EC5-SH, PI15-EC5-SH, PI16-EC5-SH, PI17-EC5-SH, PI18-EC5-SH, PI19-EC5 PI20-EC5-SH, PI1-EC10-SH, PI2-EC10-SH, PI3-EC10-SH, PI5-EC10-SH, PI7-EC10-SH, PI9-EC10-SH, PI11-EC10-SH, PI12-EC10-SH, PI13-EC10-SH, PI14-EC10-SH, PI15-EC10-SH, PI16-EC10-SH, PI17-EC10-SH, PI18-EC10-SH, PI19-EC10-SH, PI20-EC10-SH,
BK1-EC5-SH, BK3-EC5-SH, BK6-EC5-SH, BK7-EC5-SH, BK9-EC5-SH, BK11-EC5-SH, BK12-EC5-SH, BK13-EC5-SH, BK14-EC5-SH, BK15-EC5-SH, BK16-EC5-SH, BK17-EC5-SH, BK18-EC5-SH, BK19-EC5 BK20-EC5-SH, BK1-EC10-SH, BK2-EC10-SH, BK3-EC10-SH, BK5-EC10-SH, BK7-EC10-SH, BK9-EC10-SH, BK11-EC10-SH, BK12-EC10-SH, BK13-EC10-SH, BK14-EC10-SH, BK15-EC10-SH, BK16-EC10-SH, BK17-EC10-SH, BK18-EC10-SH, BK19-EC10-SH, BK20-EC10-SH,
CB1-EC5-SH, CB3-EC5-SH, CB6-EC5-SH, CB7-EC5-SH, CB9-EC5-SH, CB11-EC5-SH, CB12-EC5-SH, CB13-EC5-SH, CB14-EC5-SH, CB15-EC5-SH, CB16-EC5-SH, CB17-EC5-SH, CB18-EC5-SH, CB19-EC5 CB20-EC5-SH, CB1-EC10-SH, CB2-EC10-SH, CB3-EC10-SH, CB5-EC10-SH, CB7-EC10-SH, CB9-EC10-SH, CB11-EC10-SH, CB12-EC10-SH, CB13-EC10-SH, CB14-EC10-SH, CB15-EC10-SH, CB16-EC10-SH, CB17-EC10-SH, CB18-EC10-SH, CB19-EC10-SH, CB20-EC10-SH,

### Example 132

To a 100 ml beaker is added 40 ml of sterile water, 10 ml of 2% Chlorhexidine and 50 ml of 5% carboxymethyl cellulose salt gel, which is vigorously stirred with an overhead stirrer. Sodium hydroxide of sufficient quantity is slowly added while stirring until dissolved to provide a pH in the range of 6.5-7.5. The viscosity of the solution is between 10,000-50,000 cps at 25° C. The solution remains stable for over 48 hours. The sample obtained is C0.2-CMC2.5-SH,

In a similar manner, the following samples are prepared using the components indicated:
C1-CMC5-SBI/SBO, C3-CMC5-SBI/SBO, C6-CMC5-SBI/SBO, C7-CMC5-SBI/SBO, C9-CMC5-SBI/SBO, C11-CMC5-SBI/SBO, C12-CMC5-SBI/SBO, C13-CMC5-SBI/SBO, C14-CMC5-SBI/SBO, C15-CMC5-SBI/SBO, C16-CMC5-SBI/SBO, C17-CMC5-SBI/SBO, C18-CMC5-SBI/SBO, C19-CMC5 C20-CMC5-SBI/SBO, C1-CMC10-SBI/SBO, C2-CMC10-SBI/SBO, C3-CMC10-SBI/SBO, C5-CMC10-SBI/SBO, C7-CMC10-SBI/SBO, C9-CMC10-SBI/SBO, C11-CMC10-SBI/SBO, C12-CMC10-SBI/SBO, C13-CMC10-SBI/SBO, C14-CMC10-SBI/SBO, C15-CMC10-SBI/SBO, C16-CMC10-SBI/SBO, C17-CMC10-SBI/SBO, C18-CMC10-SBI/SBO, C19-CMC10-SBI/SBO, C20-CMC10-SBI/SBO,
PI1-CMC5-SBI/SBO, PI3-CMC5-SBI/SBO, PI6-CMC5-SBI/SBO, PI7-CMC5-SBI/SBO, PI9-CMC5-SBI/SBO, PI11-CMC5-SBI/SBO, PI12-CMC5-SBI/SBO, PI13-CMC5-SBI/SBO, PI14-CMC5-SBI/SBO, PI15-CMC5-SBI/SBO, PI16-CMC5-SBI/SBO, PI17-CMC5-SBI/SBO, PI18-CMC5-SBI/SBO, PI19-CMC5 PI20-CMC5-SBI/SBO, PI1-CMC10-SBI/SBO, PI2-CMC10-SBI/SBO, PI3-CMC10-SBI/SBO, PI5-CMC10-SBI/SBO, PI7-CMC10-SBI/SBO, PI9-CMC10-SBI/SBO, PI11-CMC10-SBI/SBO, PI12-CMC10-SBI/SBO, PI13-CMC10-SBI/SBO, PI14-CMC10-SBI/SBO, PI15-CMC10-SBI/SBO, PI16-CMC10-SBI/SBO, PI17-CMC10-SBI/SBO, PI18-CMC10-SBI/SBO, PI19-CMC10-SBI/SBO, PI20-CMC10-SBI/SBO,
BK1-CMC5-SBI/SBO, BK3-CMC5-SBI/SBO, BK6-CMC5-SBI/SBO, BK7-CMC5-SBI/SBO, BK9-CMC5-SBI/SBO, BK11-CMC5-SBI/SBO, BK12-CMC5-SBI/SBO, BK13-CMC5-SBI/SBO, BK14-CMC5-SBI/SBO, BK15-CMC5-SBI/SBO, BK16-CMC5-SBI/SBO, BK17-CMC5-SBI/SBO, BK18-CMC5-SBI/SBO, BK19-CMC5 BK20-CMC5-SBI/SBO, BK1-CMC10-SBI/SBO, BK2-CMC10-SBI/SBO, BK3-CMC10-SBI/SBO, BK5-CMC10-SBI/SBO, BK7-CMC10-SBI/SBO, BK9-CMC10-SBI/SBO, BK11-CMC10-SBI/SBO, BK12-CMC10-SBI/SBO, BK13-CMC10-SBI/SBO, BK14-CMC10-SBI/SBO, BK15-CMC10-SBI/SBO, BK16-CMC10-SBI/SBO, BK17-CMC10-SBI/SBO, BK18-CMC10-SBI/SBO, BK19-CMC10-SBI/SBO, BK20-CMC10-SBI/SBO,
CB1-CMC5-SBI/SBO, CB3-CMC5-SBI/SBO, CB6-CMC5-SBI/SBO, CB7-CMC5-SBI/SBO, CB9-CMC5-SBI/SBO, CB11-CMC5-SBI/SBO, CB12-CMC5-SBI/SBO, CB13-CMC5-SBI/SBO, CB14-CMC5-SBI/SBO, CB15-CMC5-SBI/SBO, CB16-CMC5-SBI/SBO, CB17-CMC5-SBI/SBO, CB18-CMC5-SBI/SBO, CB19-CMC5 CB20-CMC5-SBI/SBO, CB1-CMC10-SBI/SBO, CB2-CMC10-SBI/SBO, CB3-CMC10-SBI/SBO, CB5-CMC10-SBI/SBO, CB7-CMC10-SBI/SBO, CB9-CMC10-SBI/SBO, CB11-CMC10-SBI/SBO, CB12-CMC10-SBI/SBO, CB13-CMC10-SBI/SBO, CB14-CMC10-SBI/SBO, CB15-CMC10-SBI/SBO, CB16-CMC10-SBI/SBO, CB17-CMC10-SBI/SBO, CB18-CMC10-SBI/SBO, CB19-CMC10-SBI/SBO, CB20-CMC10-SBI/SBO,
C1-CMC5-SH, C3-CMC5-SH, C6-CMC5-SH, C7-CMC5-SH, C9-CMC5-SH, C11-CMC5-SH, C12-CMC5-SH, C13-CMC5-SH, C14-CMC5-SH, C15-CMC5-SH, C16-CMC5-SH, C17-CMC5-SH, C18-CMC5-SH, C19-CMC5-SH, C20-CMC5-SH, C1-CMC10-SH, C2-CMC10-SH, C3-CMC10-SH, C5-CMC10-SH, C7-CMC10-SH, C9-CMC10-SH, C11-CMC10-SH, C12-CMC10-SH, C13-CMC10-SH, C14-CMC10-SH, C15-CMC10-SH, C16-CMC10-SH, C17-CMC10-SH, C18-CMC10-SH, C19-CMC10-SH, C20-CMC10-SH,
PI1-CMC5-SH, PI3-CMC5-SH, PI6-CMC5-SH, PI7-CMC5-SH, PI9-CMC5-SH, PI11-CMC5-SH, PI12-CMC5-SH, PI13-CMC5-SH, PI14-CMC5-SH, PI15-CMC5-SH, PI16-CMC5-SH, PI17-CMC5-SH, PI18-CMC5-SH, PI19-CMC5 PI20-CMC5-SH, PI1-CMC10-SH, PI2-CMC10-SH, PI3-CMC10-SH, PI5-CMC10-SH, PI7-CMC10-SH, PI9-CMC10-SH, PI11-CMC10-SH, PI12-CMC10-SH, PI13-CMC10-SH, PI14-CMC10-SH, PI15-CMC10-SH, PI16-CMC10-SH, PI17-CMC10-SH, PI18-CMC10-SH, PI19-CMC10-SH, PI20-CMC10-SH,
BK1-CMC5-SH, BK3-CMC5-SH, BK6-CMC5-SH, BK7-CMC5-SH, BK9-CMC5-SH, BK11-CMC5-SH, BK12-CMC5-SH, BK13-CMC5-SH, BK14-CMC5-SH, BK15-CMC5-SH, BK16-CMC5-SH, BK17-CMC5-SH, BK18-CMC5-SH, BK19-CMC5 BK20-CMC5-SH, BK1-CMC10-SH, BK2-CMC10-SH, BK3-CMC10-SH, BK5-CMC10-SH, BK7-CMC10-SH, BK9-CMC10-SH, BK11-CMC10-SH, BK12-CMC10-SH, BK13-CMC10-SH, BK14-CMC10-SH, BK15-CMC10-SH, BK16-CMC10-SH, BK17-CMC10-SH, BK18-CMC10-SH, BK19-CMC10-SH, BK20-CMC10-SH,
CB1-CMC5-SH, CB3-CMC5-SH, CB6-CMC5-SH, CB7-CMC5-SH, CB9-CMC5-SH, CB11-CMC5-SH, CB12-CMC5-SH, CB13-CMC5-SH, CB14-CMC5-SH, CB15-CMC5-SH, CB16-CMC5-SH, CB17-CMC5-SH, CB18-CMC5-SH, CB19-CMC5 CB20-CMC5-SH, CB1-CMC10-SH, CB2-CMC10-SH, CB3-CMC10-SH, CB5-CMC10-SH, CB7-CMC10-SH, CB9-CMC10-SH, CB11-CMC10-SH, CB12-CMC10-SH, CB13-CMC10-SH, CB14-CMC10-SH, CB15-CMC10-SH, CB16-CMC10-SH, CB17-CMC10-SH, CB18-CMC10-SH, CB19-CMC10-SH, CB20-CMC10-SH,

### Example 133

To a 100 ml beaker is added 40 ml of sterile water, 10 ml of 2% Chlorhexidine and 50 ml of 5% methyl hydroxyethyl cellulose gel, which is vigorously stirred with an overhead stirrer. Sodium hydroxide of sufficient quantity is slowly added while stirring until dissolved to provide a pH in the range of 6.5-7.5. The viscosity of the solution is between 10,000-50,000 cps at 25° C. The solution remains stable for over 48 hours. The sample obtained is C0.2-MHEC2.5-SH,

In a similar manner, the following samples are prepared using the components indicated:
C1-MHEC5-SBI/SBO, C3-MHEC5-SBI/SBO, C6-MHEC5-SBI/SBO, C7-MHEC5-SBI/SBO, C9-MHEC5-SBI/SBO, C11-MHEC5-SBI/SBO, C12-MHEC5-SBI/SBO, C13-MHEC5-SBI/SBO, C14-MHEC5-SBI/SBO, C15-MHEC5-SBI/SBO, C16-MHEC5-SBI/SBO, C17-MHEC5-SBI/SBO, C18-MHEC5-SBI/SBO, C19-MHEC5 C20-MHEC5-SBI/SBO, C1-MHEC10-SBI/SBO, C2-MHEC10-SBI/SBO, C3-MHEC10-SBI/SBO, C5-MHEC10-SBI/SBO, C7-MHEC10-SBI/SBO, C9-MHEC10-SBI/SBO, C11-MHEC10-SBI/SBO, C12-MHEC10-SBI/SBO, C13-MHEC10-SBI/SBO, C14-MHEC10-SBI/SBO, C15-MHEC10-SBI/SBO, C16-MHEC10-SBI/SBO, C17-MHEC10-SBI/SBO, C18-MHEC10-SBI/SBO, C19-MHEC10-SBI/SBO, C20-MHEC10-SBI/SBO,
PI1-MHEC5-SBI/SBO, PI3-MHEC5-SBI/SBO, PI6-MHEC5-SBI/SBO, PI7-MHEC5-SBI/SBO, PI9-MHEC5-SBI/SBO, PI11-MHEC5-SBI/SBO, PI12-MHEC5-SBI/SBO, PI13-MHEC5-SBI/SBO, PI14-MHEC5-SBI/SBO, PI15-MHEC5-SBI/SBO, PI16-MHEC5-SBI/SBO, PI17-MHEC5-SBI/SBO, PI18-MHEC5-SBI/SBO, PI19-MHEC5 PI20-MHEC5-SBI/SBO, PI1-MHEC10-SBI/SBO, PI2-MHEC10-SBI/SBO, PI3-MHEC10-SBI/SBO, PI5-MHEC10-SBI/SBO, PI7-MHEC10-SBI/SBO, PI9-MHEC10-SBI/SBO, PI11-MHEC10-SBI/SBO, PI12-MHEC10-SBI/SBO, PI13-MHEC10-SBI/SBO, PI14-MHEC10-SBI/SBO, PI15-MHEC10-SBI/SBO, PI16-MHEC10-SBI/SBO, PI17-MHEC10-SBI/SBO, PI18-MHEC10-SBI/SBO, PI19-MHEC10-SBI/SBO, PI20-MHEC10-SBI/SBO,
BK1-MHEC5-SBI/SBO, BK3-MHEC5-SBI/SBO, BK6-MHEC5-SBI/SBO, BK7-MHEC5-SBI/SBO, BK9-MHEC5-SBI/SBO, BK11-MHEC5-SBI/SBO, BK12-MHEC5-SBI/SBO, BK13-MHEC5-SBI/SBO, BK14-MHEC5-SBI/SBO, BK15-MHEC5-SBI/SBO, BK16-MHEC5-SBI/SBO, BK17-MHEC5-SBI/SBO, BK18-MHEC5-SBI/SBO, BK19-MHEC5 BK20-MHEC5-SBI/SBO, BK1-MHEC10-SBI/SBO, BK2-MHEC10-SBI/SBO, BK3-MHEC10-SBI/SBO, BK5-MHEC10-SBI/SBO, BK7-MHEC10-SBI/SBO, BK9-MHEC10-SBI/SBO, BK11-MHEC10-SBI/SBO, BK12-MHEC10-SBI/SBO, BK13-MHEC10-SBI/SBO, BK14-MHEC10-SBI/SBO, BK15-MHEC10-SBI/SBO, BK16-MHEC10-SBI/SBO, BK17-MHEC10-SBI/SBO, BK18-MHEC10-SBI/SBO, BK19-MHEC10-SBI/SBO, BK20-MHEC10-SBI/SBO,
CB1-MHEC5-SBI/SBO, CB3-MHEC5-SBI/SBO, CB6-MHEC5-SBI/SBO, CB7-MHEC5-SBI/SBO, CB9-MHEC5-SBI/SBO, CB11-MHEC5-SBI/SBO, CB12-MHEC5-SBI/SBO, CB13-MHEC5-SBI/SBO, CB14-MHEC5-SBI/SBO, CB15-MHEC5-SBI/SBO, CB16-MHEC5-SBI/SBO, CB17-MHEC5-SBI/SBO, CB18-MHEC5-SBI/SBO, CB19-MHEC5 CB20-MHEC5-SBI/SBO, CB1-MHEC10-SBI/SBO, CB2-MHEC10-SBI/SBO, CB3-MHEC10-SBI/SBO, CB5-MHEC10-SBI/SBO, CB7-MHEC10-SBI/SBO, CB9-MHEC10-SBI/SBO, CB11-MHEC10-SBI/SBO, CB12-MHEC10-SBI/SBO, CB13-MHEC10-SBI/SBO, CB14-MHEC10-SBI/SBO, CB15-MHEC10-SBI/SBO, CB16-MHEC10-SBI/SBO, CB17-MHEC10-SBI/SBO, CB18-MHEC10-SBI/SBO, CB19-MHEC10-SBI/SBO, CB20-MHEC10-SBI/SBO,
C1-MHEC5-SH, C3-MHEC5-SH, C6-MHEC5-SH, C7-MHEC5-SH, C9-MHEC5-SH, C11-MHEC5-SH, C12-MHEC5-SH, C13-MHEC5-SH, C14-MHEC5-SH, C15-MHEC5-SH, C16-MHEC5-SH, C17-MHEC5-SH, C18-MHEC5-SH, C19-MHEC5-SH, C20-MHEC5-SH, C1-MHEC10-SH, C2-MHEC10-SH, C3-MHEC10-SH, C5-MHEC10-SH, C7-MHEC10-SH, C9-MHEC10-SH, C11-MHEC10-SH, C12-MHEC10-SH, C13-MHEC10-SH, C14-MHEC10-SH, C15-MHEC10-SH, C16-MHEC10-SH, C17-MHEC10-SH, C18-MHEC10-SH, C19-MHEC10-SH, C20-MHEC10-SH,
PI1-MHEC5-SH, PI3-MHEC5-SH, PI6-MHEC5-SH, PI7-MHEC5-SH, PI9-MHEC5-SH, PI11-MHEC5-SH, PI12-MHEC5-SH, PI13-MHEC5-SH, PI14-MHEC5-SH, PI15-MHEC5-SH, PI16-MHEC5-SH, PI17-MHEC5-SH, PI18-MHEC5-SH, PI19-MHEC5 PI20-MHEC5-SH, PI1-MHEC10-SH, PI2-MHEC10-SH, PI3-MHEC10-SH, PI5-MHEC10-SH, PI7-MHEC10-SH, PI9-MHEC10-SH, PI11-MHEC10-SH, PI12-MHEC10-SH, PI13-MHEC10-SH, PI14-MHEC10-SH, PI15-MHEC10-SH, PI16-MHEC10-SH, PI17-MHEC10-SH, PI18-MHEC10-SH, PI19-MHEC10-SH, PI20-MHEC10-SH,
BK1-MHEC5-SH, BK3-MHEC5-SH, BK6-MHEC5-SH, BK7-MHEC5-SH, BK9-MHEC5-SH, BK11-MHEC5-SH, BK12-MHEC5-SH, BK13-MHEC5-SH, BK14-MHEC5-SH, BK15-MHEC5-SH, BK16-MHEC5-SH, BK17-MHEC5-SH, BK18-MHEC5-SH, BK19-MHEC5 BK20-MHEC5-SH, BK1-MHEC10-SH, BK2-MHEC10-SH, BK3-MHEC10-SH, BK5MHEC10-SH, BK7-MHEC10-SH, BK9-MHEC10-SH, BK11-MHEC10-SH, BK12-MHEC10-SH, BK13-MHEC10-SH, BK14-MHEC10-SH, BK15-MHEC10-SH, BK16-MHEC10-SH, BK17-MHEC10-SH, BK18-MHEC10-SH, BK19-MHEC10-SH, BK20-MHEC10-SH,
CB1-MHEC5-SH, CB3-MHEC5-SH, CB6-MHEC5-SH, CB7-MHEC5-SH, CB9-MHEC5-SH, CB11-MHEC5-SH, CB12-MHEC5-SH, CB13-MHEC5-SH, CB14-MHEC5-SH, CB15-MHEC5-SH, CB16-MHEC5-SH, CB17-MHEC5-SH, CB18-MHEC5-SH, CB19-MHEC5 CB20-MHEC5-SH, CB1-MHEC10-SH, CB2-MHEC10-SH, CB3-MHEC10-SH, CB5-MHEC10-SH, CB7-MHEC10-SH, CB9-MHEC10-SH, CB11-MHEC10-SH, CB12-MHEC10-SH, CB13-MHEC10-SH, CB14-MHEC10-SH, CB15-MHEC10-SH, CB16-MHEC10-SH, CB17-MHEC10-SH, CB18-MHEC10-SH, CB19-MHEC10-SH, CB20-MHEC10-SH,

### Example 134

To a 100 ml beaker is added 40 ml of sterile water, 10 ml of 2% Chlorhexidine and 50 ml of 5% methyl hydroxyethyl cellulose gel, which is vigorously stirred with an overhead stirrer. Sodium hydroxide of sufficient quantity is slowly added while stirring until dissolved to provide a pH in the range of 6.5-7.5. The viscosity of the solution is between 10,000-50,000 cps at 25° C. The solution remains stable for over 48 hours. The sample obtained is C0.2-MHEC2.5-SH,

In a similar manner, the following samples are prepared using the components indicated:
C1-MHEC5-SBI/SBO, C3-MHEC5-SBI/SBO, C6-MHEC5-SBI/SBO, C7-MHEC5-SBI/SBO, C9-MHEC5-SBI/SBO, C11-MHEC5-SBI/SBO, C12-MHEC5-SBI/SBO, C13-MHEC5-SBI/SBO, C14-MHEC5-SBI/SBO, C15-MHEC5-SBI/SBO, C16-MHEC5-SBI/SBO,

C17-MHEC5-SBI/SBO, C18-MHEC5-SBI/SBO, C19-MHEC5 C20-MHEC5-SBI/SBO, C1-MHEC10-SBI/SBO, C2-MHEC10-SBI/SBO, C3-MHEC10-SBI/SBO, C5-MHEC10-SBI/SBO, C7-MHEC10-SBI/SBO, C9-MHEC10-SBI/SBO, C11-MHEC10-SBI/SBO, C12-MHEC10-SBI/SBO, C13-MHEC10-SBI/SBO, C14-MHEC10-SBI/SBO, C15-MHEC10-SBI/SBO, C16-MHEC10-SBI/SBO, C17-MHEC10-SBI/SBO, C18-MHEC10-SBI/SBO, C19-MHEC10-SBI/SBO, C20-MHEC10-SBI/SBO,
PI1-MHEC5-SBI/SBO, PI3-MHEC5-SBI/SBO, PI6-MHEC5-SBI/SBO, PI7-MHEC5-SBI/SBO, PI9-MHEC5-SBI/SBO, PI11-MHEC5-SBI/SBO, PI12-MHEC5-SBI/SBO, PI13-MHEC5-SBI/SBO, PI14-MHEC5-SBI/SBO, PI15-MHEC5-SBI/SBO, PI16-MHEC5-SBI/SBO, PI17-MHEC5-SBI/SBO, PI18-MHEC5-SBI/SBO, PI19-MHEC5 PI20-MHEC5-SBI/SBO, PI1-MHEC10-SBI/SBO, PI2-MHEC10-SBI/SBO, PI3-MHEC10-SBI/SBO, PI5-MHEC10-SBI/SBO, PI7-MHEC10-SBI/SBO, PI9-MHEC10-SBI/SBO, PI11-MHEC10-SBI/SBO, PI12-MHEC10-SBI/SBO, PI13-MHEC10-SBI/SBO, PI14-MHEC10-SBI/SBO, PI15-MHEC10-SBI/SBO, PI16-MHEC10-SBI/SBO, PI17-MHEC10-SBI/SBO, PI18-MHEC10-SBI/SBO, PI19-MHEC10-SBI/SBO, PI20-MHEC10-SBI/SBO,
BK1-MHEC5-SBI/SBO, BK3-MHEC5-SBI/SBO, BK6-MHEC5-SBI/SBO, BK7-MHEC5-SBI/SBO, BK9-MHEC5-SBI/SBO, BK11-MHEC5-SBI/SBO, BK12-MHEC5-SBI/SBO, BK13-MHEC5-SBI/SBO, BK14-MHEC5-SBI/SBO, BK15-MHEC5-SBI/SBO, BK16-MHEC5-SBI/SBO, BK17-MHEC5-SBI/SBO, BK18-MHEC5-SBI/SBO, BK19-MHEC5 BK20-MHEC5-SBI/SBO, BK1-MHEC10-SBI/SBO, BK2-MHEC10-SBI/SBO, BK3-MHEC10-SBI/SBO, BK5-MHEC10-SBI/SBO, BK7-MHEC10-SBI/SBO, BK9-MHEC10-SBI/SBO, BK11-MHEC10-SBI/SBO, BK12-MHEC10-SBI/SBO, BK13-MHEC10-SBI/SBO, BK14-MHEC10-SBI/SBO, BK15-MHEC10-SBI/SBO, BK16-MHEC10-SBI/SBO, BK17-MHEC10-SBI/SBO, BK18-MHEC10-SBI/SBO, BK19-MHEC10-SBI/SBO, BK20-MHEC10-SBI/SBO,
CB1-MHEC5-SBI/SBO, CB3-MHEC5-SBI/SBO, CB6-MHEC5-SBI/SBO, CB7-MHEC5-SBI/SBO, CB9-MHEC5-SBI/SBO, CB11-MHEC5-SBI/SBO, CB12-MHEC5-SBI/SBO, CB13-MHEC5-SBI/SBO, CB14-MHEC5-SBI/SBO, CB15-MHEC5-SBI/SBO, CB16-MHEC5-SBI/SBO, CB17-MHEC5-SBI/SBO, CB18-MHEC5-SBI/SBO, CB19-MHEC5 CB20-MHEC5-SBI/SBO, CB1-MHEC10-SBI/SBO, CB2-MHEC10-SBI/SBO, CB3-MHEC10-SBI/SBO, CB5-MHEC10-SBI/SBO, CB7-MHEC10-SBI/SBO, CB9-MHEC10-SBI/SBO, CB11-MHEC10-SBI/SBO, CB12-MHEC10-SBI/SBO, CB13-MHEC10-SBI/SBO, CB14-MHEC10-SBI/SBO, CB15-MHEC10-SBI/SBO, CB16-MHEC10-SBI/SBO, CB17-MHEC10-SBI/SBO, CB18-MHEC10-SBI/SBO, CB19-MHEC10-SBI/SBO, CB20-MHEC10-SBI/SBO,
C1-MHEC5-SH, C3-MHEC5-SH, C6-MHEC5-SH, C7-MHEC5-SH, C9-MHEC5-SH, C11-MHEC5-SH, C12-MHEC5-SH, C13-MHEC5-SH, C14-MHEC5-SH, C15-MHEC5-SH, C16-MHEC5-SH, C17-MHEC5-SH, C18-MHEC5-SH, C19-MHEC5-SH, C20-MHEC5-SH, C1-MHEC10-SH, C2-MHEC10-SH, C3-MHEC10-SH, C5-MHEC10-SH, C7-MHEC10-SH, C9-MHEC10-SH, C11-MHEC10-SH, C12-MHEC10-SH, C13-MHEC10-SH, C14-MHEC10-SH, C15-MHEC10-SH, C16-MHEC10-SH, C17-MHEC10-SH, C18-MHEC10-SH, C19-MHEC10-SH, C20-MHEC10-SH,
PI1-MHEC5-SH, PI3-MHEC5-SH, PI6-MHEC5-SH, PI7-MHEC5-SH, PI9-MHEC5-SH, PI11-MHEC5-SH, PI12-MHEC5-SH, PI13-MHEC5-SH, PI14-MHEC5-SH, PI15-MHEC5-SH, PI16-MHEC5-SH, PI17-MHEC5-SH, PI18-MHEC5-SH, PI19-MHEC5 PI20-MHEC5-SH, PI1-MHEC10-SH, PI2-MHEC10-SH, PI3-MHEC10-SH, PI5-MHEC10-SH, PI7-MHEC10-SH, PI9-MHEC10-SH, PI11-MHEC10-SH, PI12-MHEC10-SH, PI13-MHEC10-SH, PI14-MHEC10-SH, PI15-MHEC10-SH, PI16-MHEC10-SH, PI17-MHEC10-SH, PI18-MHEC10-SH, PI19-MHEC10-SH, PI20-MHEC10-SH,
BK1-MHEC5-SH, BK3-MHEC5-SH, BK6-MHEC5-SH, BK7-MHEC5-SH, BK9-MHEC5-SH, BK11-MHEC5-SH, BK12-MHEC5-SH, BK13-MHEC5-SH, BK14-MHEC5-SH, BK15-MHEC5-SH, BK16-MHEC5-SH, BK17-MHEC5-SH, BK18-MHEC5-SH, BK19-MHEC5 BK20-MHEC5-SH, BK1-MHEC10-SH, BK2-MHEC10-SH, BK3-MHEC10-SH, BK5-MHEC10-SH, BK7-MHEC10-SH, BK9-MHEC10-SH, BK11-MHEC10-SH, BK12-MHEC10-SH, BK13-MHEC10-SH, BK14-MHEC10-SH, BK15-MHEC10-SH, BK16-MHEC10-SH, BK17-MHEC10-SH, BK18-MHEC10-SH, BK19-MHEC10-SH, BK20-MHEC10-SH,
CB1-MHEC5-SH, CB3-MHEC5-SH, CB6-MHEC5-SH, CB7-MHEC5-SH, CB9-MHEC5-SH, CB11-MHEC5-SH, CB12-MHEC5-SH, CB13-MHEC5-SH, CB14-MHEC5-SH, CB15-MHEC5-SH, CB16-MHEC5-SH, CB17-MHEC5-SH, CB18-MHEC5-SH, CB19-MHEC5 CB20-MHEC5-SH, CB1-MHEC10-SH, CB2-MHEC10-SH, CB3-MHEC10-SH, CB5-MHEC10-SH, CB7-MHEC10-SH, CB9-MHEC10-SH, CB11-MHEC10-SH, CB12-MHEC10-SH, CB13-MHEC10-SH, CB14-MHEC10-SH, CB15-MHEC10-SH, CB16-MHEC10-SH, CB17-MHEC10-SH, CB18-MHEC10-SH, CB19-MHEC10-SH, CB20-MHEC10-SH,

### Example 135

To a 100 ml beaker is added 40 ml of sterile water, 10 ml of 2% Chlorhexidine and 50 ml of 5% hydroxyethyl cellulose gel, which is vigorously stirred with an overhead stirrer. Sodium hydroxide of sufficient quantity is slowly added while stirring until dissolved to provide a pH in the range of 6.5-7.5. The viscosity of the solution is between 10,000-50,000 cps at 25° C. The solution remains stable for over 48 hours. The sample obtained is C0.2-HEC2.5-SH,

In a similar manner, the following samples are prepared using the components indicated:
C1-HEC5-SBI/SBO, C3-HEC5-SBI/SBO, C6-HEC5-SBI/SBO, C7-HEC5-SBI/SBO, C9-HEC5-SBI/SBO, C11-HEC5-SBI/SBO, C12-HEC5-SBI/SBO, C13-HEC5-SBI/SBO, C14-HEC5-SBI/SBO, C15-HEC5-SBI/SBO, C16-HEC5-SBI/SBO, C17-HEC5-SBI/SBO, C18-HEC5-SBI/SBO, C19-HEC5 C20-HEC5-SBI/SBO, C1-HEC10-SBI/SBO, C2-HEC10-SBI/SBO, C3-HEC10-SBI/SBO, C5-HEC10-SBI/SBO, C7-HEC10-SBI/SBO, C9-HEC10-SBI/SBO, C11-HEC10-SBI/SBO, C12-HEC10-SBI/SBO, C13-HEC10-SBI/SBO, C14-HEC10-SBI/SBO, C15-HEC10-SBI/SBO, C16-HEC10-SBI/SBO, C17-HEC10-SBI/SBO, C18-HEC10-SBI/SBO, C19-HEC10-SBI/SBO, C20-HEC10-SBI/SBO,
PI1-HEC5-SBI/SBO, PI3-HEC5-SBI/SBO, PI6-HEC5-SBI/SBO, PI7-HEC5-SBI/SBO, PI9-HEC5-SBI/SBO, PI11-HEC5-SBI/SBO, PI12-HEC5-SBI/SBO, PI13-HEC5-SBI/SBO, PI14-HEC5-SBI/SBO, PI15-HEC5-SBI/SBO, PI16-HEC5-SBI/SBO, PI17-HEC5-SBI/SBO, PI18-HEC5-SBI/SBO, PI19-HEC5 PI20-HEC5-SBI/SBO, PI1-HEC10-SBI/SBO, PI2-HEC10-SBI/SBO, PI3-HEC10-SBI/SBO, PI5-HEC10-SBI/SBO, PI7-HEC10-SBI/SBO, PI9-HEC10-SBI/SBO, PI11-HEC10-SBI/SBO, PI12-HEC10-SBI/SBO, PI13-HEC10-SBI/SBO, PI14-HEC10-SBI/SBO, PI15-HEC10-SBI/SBO, PI16-HEC10-SBI/SBO, PI17-HEC10-SBI/SBO, PI18-HEC10-SBI/SBO, PI19-HEC10-SBI/SBO, PI20-HEC10-SBI/SBO,
BK1-HEC5-SBI/SBO, BK3-HEC5-SBI/SBO, BK6-HEC5-SBI/SBO, BK7-HEC5-SBI/SBO, BK9-HEC5-SBI/SBO, BK11-HEC5-SBI/SBO, BK12-HEC5-SBI/SBO, BK13-HEC5-SBI/SBO, BK14-HEC5-SBI/SBO, BK15-HEC5-SBI/SBO, BK16-HEC5-SBI/SBO, BK17-HEC5-SBI/SBO, BK18-HEC5-SBI/SBO, BK19-HEC5 BK20-HEC5-SBI/SBO, BK1-HEC10-SBI/SBO, BK2-HEC10-SBI/SBO, BK3-HEC10-SBI/SBO, BK5-HEC10-SBI/SBO, BK7-HEC10-SBI/SBO, BK9-HEC10-SBI/SBO, BK11-HEC10-SBI/SBO, BK12-HEC10-SBI/SBO, BK13-HEC10-SBI/SBO, BK14-HEC10-SBI/SBO, BK15-HEC10-SBI/SBO, BK16-HEC10-SBI/SBO, BK17-HEC10-SBI/SBO, BK18-HEC10-SBI/SBO, BK19-HEC10-SBI/SBO, BK20-HEC10-SBI/SBO,
CB1-HEC5-SBI/SBO, CB3-HEC5-SBI/SBO, CB6-HEC5-SBI/SBO, CB7-HEC5-SBI/SBO, CB9-HEC5-SBI/SBO, CB11-HEC5-SBI/SBO, CB12-HEC5-SBI/SBO, CB13-HEC5-SBI/SBO, CB14-HEC5-SBI/SBO, CB15-HEC5-SBI/SBO, CB16-HEC5-SBI/SBO, CB17-HEC5-SBI/SBO, CB18-HEC5-SBI/SBO, CB19-HEC5 CB20-HEC5-SBI/SBO, CB1-HEC10-SBI/SBO, CB2-HEC10-SBI/SBO, CB3-HEC10-SBI/SBO, CB5-HEC10-SBI/SBO, CB7-HEC10-SBI/SBO, CB9-HEC10-SBI/SBO, CB11-HEC10-SBI/SBO, CB12-HEC10-SBI/SBO, CB13-HEC10-SBI/SBO, CB14-HEC10-SBI/SBO, CB15-HEC10-SBI/SBO, CB16-HEC10-SBI/SBO, CB17-HEC10-SBI/SBO, CB18-HEC10-SBI/SBO, CB19-HEC10-SBI/SBO, CB20-HEC10-SBI/SBO,
C1-HEC5-SH, C3-HEC5-SH, C6-HEC5-SH, C7-HEC5-SH, C9-HEC5-SH, C11-HEC5-SH, C12-HEC5-SH, C13-HEC5-SH, C14-HEC5-SH, C15-HEC5-SH, C16-HEC5-SH, C17-HEC5-SH, C18-HEC5-SH, C19-HEC5-SH, C20-HEC5-SH, C1-HEC10-SH, C2-HEC10-SH, C3-HEC10-SH, C5-HEC10-SH, C7-HEC10-SH, C9-HEC10-SH, C11-HEC10-SH, C12-HEC10-SH, C13-HEC10-SH, C14-HEC10-SH, C15-HEC10-SH, C16-HEC10-SH, C17-HEC10-SH, C18-HEC10-SH, C19-HEC10-SH, C20-HEC10-SH,
PI1-HEC5-SH, PI3-HEC5-SH, PI6-HEC5-SH, PI7-HEC5-SH, PI9-HEC5-SH, PI11-HEC5-SH, PI12-HEC5-SH, PI13-HEC5-SH, PI14-HEC5-SH, PI15-HEC5-SH, PI16-HEC5-SH, PI17-HEC5-SH, PI18-HEC5-SH, PI19-HEC5 PI20-HEC5-SH, PI1-HEC10-SH, PI2-HEC10-SH, PI3-HEC10-SH, PI5-HEC10-SH, PI7-HEC10-SH, PI9-HEC10-SH, PI11-HEC10-SH, PI12-HEC10-SH, PI13-HEC10-SH, PI14-HEC10-SH, PI15-HEC10-SH, PI16-HEC10-SH, PI17-HEC10-SH, PI18-HEC10-SH, PI19-HEC10-SH, PI20-HEC10-SH,
BK1-HEC5-SH, BK3-HEC5-SH, BK6-HEC5-SH, BK7-HEC5-SH, BK9-HEC5-SH, BK11-HEC5-SH, BK12-HEC5-SH, BK13-HEC5-SH, BK14-HEC5-SH, BK15-HEC5-SH, BK16-HEC5-SH, BK17-HEC5-SH, BK18-HEC5-SH, BK19-HEC5 BK20-HEC5-SH, BK1-HEC10-SH, BK2-HEC10-SH, BK3-HEC10-SH, BK5-HEC10-SH, BK7-HEC10-SH, BK9-HEC10-SH, BK11-HEC10-SH, BK12-HEC10-SH, BK13-HEC10-SH, BK14-HEC10-SH, BK15-HEC10-SH, BK16-HEC10-SH, BK17-HEC10-SH, BK18-HEC10-SH, BK19-HEC10-SH, BK20-HEC10-SH,
CB1-HEC5-SH, CB3-HEC5-SH, CB6-HEC5-SH, CB7-HEC5-SH, CB9-HEC5-SH, CB11-HEC5-SH, CB12-HEC5-SH, CB13-HEC5-SH, CB14-HEC5-SH, CB15-HEC5-SH, CB16-HEC5-SH, CB17-HEC5-SH, CB18-HEC5-SH, CB19-HEC5 CB20-HEC5-SH, CB1-HEC10-SH, CB2-HEC10-SH, CB3-HEC10-SH, CB5-HEC10-SH, CB7-HEC10-SH, CB9-HEC10-SH, CB11-HEC10-SH, CB12-HEC10-SH, CB13-HEC10-SH, CB14-HEC10-SH, CB15-HEC10-SH, CB16-HEC10-SH, CB17-HEC10-SH, CB18-HEC10-SH, CB19-HEC10-SH, CB20-HEC10-SH,

### Example 136

To a 100 ml beaker is added 40 ml of sterile water, 10 ml of 2% Chlorhexidine and 50 ml of 5% polyvinyl alcohol gel, which is vigorously stirred with an overhead stirrer. Sodium hydroxide of sufficient quantity is slowly added while stirring until dissolved to provide a pH in the range of 6.5-7.5. The viscosity of the solution is between 10,000-50,000 cps at 25° C. The solution remains stable for over 48 hours. The sample obtained is C0.2-PVA2.5-SH,

In a similar manner, the following samples are prepared using the components indicated:
C1-PVA5-SBI/SBO, C3-PVA5-SBI/SBO, C6-PVA5-SBI/SBO, C7-PVA5-SBI/SBO, C9-PVA5-SBI/SBO, C11-PVA5-SBI/SBO, C12-PVA5-SBI/SBO, C13-PVA5-SBI/SBO, C14-PVA5-SBI/SBO, C15-PVA5-SBI/SBO, C16-PVA5-SBI/SBO, C17-PVA5-SBI/SBO, C18-PVA5-SBI/SBO, C19-PVA5 C20-PVA5-SBI/SBO, C1-PVA10-SBI/SBO, C2-PVA10-SBI/SBO, C3-PVA10-SBI/SBO, C5-PVA10-SBI/SBO, C7-PVA10-SBI/SBO, C9-PVA10-SBI/SBO, C11-PVA10-SBI/SBO, C12-PVA10-SBI/SBO, C13-PVA10-SBI/SBO, C14-PVA10-SBI/SBO, C15-PVA10-SBI/SBO, C16-PVA10-SBI/SBO, C17-PVA10-SBI/SBO, C18-PVA10-SBI/SBO, C19-PVA10-SBI/SBO, C20-PVA10-SBI/SBO,
PI1-PVA5-SBI/SBO, PI3-PVA5-SBI/SBO, PI6-PVA5-SBI/SBO, PI7-PVA5-SBI/SBO, PI9-PVA5-SBI/SBO, PI11-PVA5-SBI/SBO, PI12-PVA5-SBI/SBO, PI13-PVA5-SBI/SBO, PI14-PVA5-SBI/SBO, PI15-PVA5-SBI/SBO, PI16-PVA5-SBI/SBO, PI17-PVA5-SBI/SBO, PI18-PVA5-SBI/SBO, PI19-PVA5 PI20-PVA5-SBI/SBO, PI1-PVA10-SBI/SBO, PI2-PVA10SBI/SBO, PI3-PVA10-SBI/SBO, PI5-PVA10-SBI/SBO, PI7-PVA10-SBI/SBO, PI9-PVA10-SBI/SBO, PI11-PVA10-SBI/SBO, PI12-PVA10-SBI/SBO, PI13-PVA10-SBI/SBO, PI14-PVA10-SBI/SBO, PI15-PVA10-SBI/SBO, PI16-PVA10-SBI/SBO, PI17-PVA10-SBI/SBO, PI18-PVA10-SBI/SBO, PI19-PVA10-SBI/SBO, PI20-PVA10-SBI/SBO,
BK1-PVA5-SBI/SBO, BK3-PVA5-SBI/SBO, BK6-PVA5-SBI/SBO, BK7-PVA5-SBI/SBO, BK9-PVA5-SBI/SBO, BK11-PVA5-SBI/SBO, BK12-PVA5-SBI/SBO, BK13-PVA5-SBI/SBO, BK14-PVA5-SBI/SBO, BK15-PVA5-SBI/SBO, BK16-PVA5-SBI/SBO, BK17-PVA5-SBI/SBO, BK18-PVA5-SBI/SBO, BK19-PVA5 BK20-PVA5-SBI/SBO, BK1-PVA10-SBI/SBO, BK2-PVA10-SBI/SBO, BK3-PVA10-SBI/SBO, BK5-PVA10-SBI/SBO, BK7-PVA10-SBI/SBO, BK9-PVA10-SBI/SBO, BK11-PVA10-SBI/SBO, BK12-PVA10-SBI/SBO, BK13-PVA10-SBI/SBO, BK14-PVA10-SBI/SBO, BK15-PVA10-SBI/SBO, BK16-PVA10-SBI/SBO, BK17-PVA10-SBI/SBO, BK18-PVA10-SBI/SBO, BK19-PVA10-SBI/SBO, BK20-PVA10-SBI/SBO,
CB1-PVA5-SBI/SBO, CB3-PVA5-SBI/SBO, CB6-PVA5-SBI/SBO, CB7-PVA5-SBI/SBO, CB9-PVA5-SBI/SBO, CB11-PVA5-SBI/SBO, CB12-PVA5-SBI/SBO, CB13-PVA5-SBI/SBO, CB14-PVA5-SBI/SBO, CB15-PVA5-SBI/SBO, CB16-PVA5-SBI/SBO, CB17-PVA5-SBI/SBO, CB18-PVA5-SBI/SBO, CB19-PVA5 CB20-PVA5-SBI/SBO, CB1-PVA10-SBI/SBO, CB2-PVA10-SBI/SBO, CB3-PVA10-SBI/SBO, CB5-PVA10-SBI/SBO, CB7-PVA10-SBI/SBO, CB9-PVA10-SBI/SBO, CB11-PVA10-SBI/SBO, CB12-PVA10-SBI/SBO, CB13-PVA10-SBI/SBO, CB14-PVA10-SBI/SBO, CB15-PVA10-SBI/SBO, CB16-PVA10-SBI/SBO, CB17-PVA10-SBI/SBO, CB18-PVA10-SBI/SBO, CB19-PVA10-SBI/SBO, CB20-PVA10-SBI/SBO,
C1-PVA5-SH, C3-PVA5-SH, C6-PVA5-SH, C7-PVA5-SH, C9-PVA5-SH, C11-PVA5-SH, C12-PVA5-SH, C13-PVA5-SH, C14-PVA5-SH, C15-PVA5-SH, C16-PVA5-SH, C17-PVA5-SH, C18-PVA5-SH, C19-PVA5-SH, C20-PVA5-SH, C1-PVA10-SH, C2-PVA10-SH, C3-PVA10-SH, C5-PVA10-SH, C7-PVA10-SH, C9-PVA10-SH, C11-PVA10-SH, C12-PVA10-SH, C13-PVA10-SH, C14-PVA10-SH, C15-PVA10-SH, C16-PVA10-SH, C17-PVA10-SH, C18-PVA10-SH, C19-PVA10-SH, C20-PVA10-SH,
PI1-PVA5-SH, PI3-PVA5-SH, PI6-PVA5-SH, PI7-PVA5-SH, PI9-PVA5-SH, PI11-PVA5-SH, PI12-PVA5-SH, PI13-PVA5-SH, PI14-PVA5-SH, PI15-PVA5-SH, PI16-PVA5-SH, PI17-PVA5-SH, PI18-PVA5-SH, PI19-PVA5 PI20-PVA5-SH, PI1-PVA10-SH, PI2-PVA10-SH, PI3-PVA10-SH, PI5-PVA10-SH, PI7-PVA10-SH, PI9-PVA10-SH, PI11-PVA10-SH, PI12-PVA10-SH, PI13-PVA10-SH, PI14-PVA10-SH, PI15-PVA10-SH, PI16-PVA10-SH, PI17-PVA10-SH, PI18-PVA10-SH, PI19-PVA10-SH, PI20-PVA10-SH,
BK1-PVA5-SH, BK3-PVA5-SH, BK6-PVA5-SH, BK7-PVA5-SH, BK9-PVA5-SH, BK11-PVA5-SH, BK12-PVA5-SH, BK13-PVA5-SH, BK14-PVA5-SH, BK15-PVA5-SH, BK16-PVA5-SH, BK17-PVA5-SH, BK18-PVA5-SH, BK19-PVA5 BK20-PVA5-SH, BK1-PVA10-SH, BK2-PVA10-SH, BK3-PVA10-SH, BK5-PVA10-SH, BK7-PVA10-SH, BK9-PVA10-SH, BK11-PVA10-SH, BK12-PVA10-SH, BK13-PVA10-SH, BK14-PVA10-SH, BK15-PVA10-SH, BK16-PVA10-SH, BK17-PVA10-SH, BK18-PVA10-SH, BK19-PVA10-SH, BK20-PVA10-SH,
CB1-PVA5-SH, CB3-PVA5-SH, CB6-PVA5-SH, CB7-PVA5-SH, CB9-PVA5-SH, CB11-PVA5-SH, CB12-PVA5-SH, CB13-PVA5-SH, CB14-PVA5-SH, CB15-PVA5-SH, CB16-PVA5-SH, CB17-PVA5-SH, CB18-PVA5-SH, CB19-PVA5 CB20-PVA5-SH, CB1-PVA10-SH, CB2-PVA10-SH, CB3-PVA10-SH, CB5-PVA10-SH, CB7-PVA10-SH, CB9-PVA10-SH, CB11-PVA10-SH, CB12-PVA10-SH, CB13-PVA10-SH, CB14-PVA10-SH, CB15-PVA10-SH, CB16-PVA10-SH, CB17-PVA10-SH, CB18-PVA10-SH, CB19-PVA10-SH, CB20-PVA 10-SH,

Each sample will have a viscosity between 10,000-50,000 cps at 25° C. and a pH in the range of 6.0-7.0. These solutions will remain stable for over 14 days. All sample solutions will be isotonic having an osmolarity in the range of 100-450 mOsmol/L.

Preferably the components of the preparations are sterile and the sample solutions prepared are packaged and stored to maintain sterility.

It may be desirable to adjust the osmolarity of a sample solution to approximate that of tears, which is typically about 300 mOsmol/L. Water is added to the solution or its components to reduce osmolarity when desired and sodium chloride is preferably added to the solution or its components to increase osmolarity when desired. Where the use of sodium chloride is necessary to increase the osmolarity of the 100 ml solutions described above, about 0.9 gm (about 0.9%) of sodium chloride will typically be sufficient to do so. The salt can be added to the sample solution before or after the other components of the buffered antiseptic solution.

For the samples above, the abbreviations represent the following compounds:
C=Chlorhexidine,
PI=povidone-iodine,
BK=benzalkonium chloride,
CB-Chlorobutanol,
SH=sodium hydroxide
SBI=sodium bicarbonate
SBO=sodium borate
MC=methylcellulose gel
P=poloxamer gel,
HPC=hydroxypropyl cellulose gel,
MHPC=methyl hydroxypropyl cellulose gel,
HPMC=hydroxypropyl methyl cellulose gel,
CA=cellulose acetate gel,
EC=ethyl cellulose gel,
CMC=carboxymethyl cellulose salt gel,
MHEC=methyl hydroxyethyl cellulose gel,
HEC=hydroxyethyl cellulose gel, and
PVA=polyvinyl alcohol gel.

The number following each abbreviation is its weight percent (wt %) in solution.

Additives that can be optionally introduced to these sample solutions in effective amounts include steroids, antibiotics, antioxidants, preservatives, solubilizing agents, dispersants and acetic acid.

### Preparation of Buffered Anatomic Solution w/Gel and Anesthetic Agent

The following solutions are preferably prepared just prior to use because of stability issues. Preferably these solutions are prepared from kits where the anesthetic can be added to the buffered anatomic solution just before use.

### Example 137a and 137b

a) To a 100 ml beaker is added 35.4 ml of sterile water, 10 ml of 2% Chlorhexidine and 50 ml of 5% methylcellulose gel, which is vigorously stirred with an overhead stirrer. Sodium bicarbonate of sufficient quantity and sodium borate 2.6 gm are slowly added while stirring until dissolved to provide a pH in the range of 6.0-7.0. Two gram of 2% Lidocaine is added following the addition of buffer. The viscosity of the solution is between 10,000-50,000 cps at 25° C. The solution remains stable for over 48 hours. The sample obtained is C0.2-MC2.5-SBI/SBO-L.
b) To a 100 ml beaker is added 35.4 ml of sterile water, 10 ml of 2% Chlorhexidine and 50 ml of 5% methylcellulose gel, which is vigorously stirred with an overhead stirrer. Sodium bicarbonate of sufficient quantity and sodium borate 2.6 gm are slowly added while stirring until dissolved to provide a pH in the range of 6.0-7.0. Two gram of 2% tetracaine is added following the addition of buffer. The viscosity of the solution is between 10,000-50,000 cps at 25° C. The solution remains stable for over 48 hours. The sample obtained is C0.2-MC2.5-SBI/SBO-T.

### Examples 138a and 138b

a) To a 100 ml beaker is added 25.4 ml of sterile water, 20 ml of 4 wt % Chlorhexidine and 50 ml of 5 5% methylcellulose gel, which is vigorously stirred with an overhead stirrer. Sodium bicarbonate of sufficient quantity and sodium borate 2.6 gm are slowly added while stirring until dissolved to provide a pH in the range of 6.0-7.0. Two gram of 2% Lidocaine is added following the addition of buffer. The viscosity of the solution is between 10,000-50,000 cps at 25° C. The solution remains stable for over 48 hours. The sample obtained is C0.8-MC2.5-SBI/SBO-L.
b) To a 100 ml beaker is added 25.4 ml of sterile water, 20 ml of 4 wt % Chlorhexidine and 50 ml of 5 5% methylcellulose gel, which is vigorously stirred with an overhead stirrer. Sodium bicarbonate of sufficient quantity and sodium borate 2.6 gm are slowly added while stirring until dissolved to provide a pH in the range of 6.0-7.0. Two gram of 2% narcaine is added following the addition of buffer. The viscosity of the solution is between 10,000-50,000 cps at 25° C. The solution remains stable for over 48 hours. The sample obtained is C0.8-MC2.5-SBI/SBO-N.

### Example 139a and 139b

a) To a 100 ml beaker is added 45.4 ml of sterile water, 40 ml of 4% Chlorhexidine and 10 ml of 1% methylcellulose gel, which is vigorously stirred with an overhead stirrer. Sodium bicarbonate of sufficient quantity and sodium borate 2.6 gm are slowly added while stirring until dissolved to provide a pH in the range of 6.0-7.0. Two gram of 2% Lidocaine is added following the addition of buffer. The viscosity of the solution is between 10,000-50,000 cps at 25° C. The solution remains stable for over 48 hours. The sample obtained is C1.6-MC0.1-SBI/SBO-L.
b) To a 100 ml beaker is added 45.4 ml of sterile water, 40 ml of 4% Chlorhexidine and 10 ml of 1% methylcellulose gel, which is vigorously stirred with an overhead stirrer. Sodium bicarbonate of sufficient quantity and sodium borate 2.6 gm are slowly added while stirring until dissolved to provide a pH in the range of 6.0-7.0. Two gram of 2% mepivicaine is added following the addition of buffer. The viscosity of the solution is between 10,000-50,000 cps at 25° C. The solution remains stable for over 48 hours. The sample obtained is C1.6-MC0.1-SBI/SBO-M.

### Example 140a and 140b

a) To a 100 ml beaker is added 15.4 ml of sterile water, 30 ml of 6% Chlorhexidine and 50 ml of 5% methylcellulose gel, which is vigorously stirred with an overhead stirrer. Sodium bicarbonate of sufficient quantity and sodium borate 2.6 gm are slowly added while stirring until dissolved to provide a pH in the range of 6.0-7.0. Two gram of 2% Lidocaine is added following the addition of buffer. The viscosity of the solution is between 10,000-50,000 cps at 25° C. The solution remains stable for over 48 hours. The sample obtained is C1.8-MC2.5-SBI/SBO-L.
b) To a 100 ml beaker is added 15.4 ml of sterile water, 30 ml of 6% Chlorhexidine and 50 ml of 5% methylcellulose gel, which is vigorously stirred with an overhead stirrer. Sodium bicarbonate of sufficient quantity and sodium borate 2.6 gm are slowly added while stirring until dissolved to provide a pH in the range of 6.0-7.0. Two gram of 2% proparacaine is added following the addition of buffer. The viscosity of the solution is between 10,000-50,000 cps at 25° C. The solution remains stable for over 48 hours. The sample obtained is C1.8-MC2.5-SBI/SBO-PRO.

### Example 141a and 141b

a) To a 100 ml beaker is added 25.4 ml of sterile water, 50 ml of 10% Chlorhexidine and 20 ml of 2% methylcellulose gel, which is vigorously stirred with an overhead stirrer. Sodium bicarbonate of sufficient quantity and sodium borate 2.6 gm are slowly added while stirring until dissolved to provide a pH in the range of 6.0-7.0. Two gram of 2% Lidocaine is added following the addition of buffer. The viscosity of the solution is between 10,000-50,000 cps at 25° C. The solution remains stable for over 48 hours. The sample obtained is C5-MC0.4-SBI/SBO-L.
b) To a 100 ml beaker is added 25.4 ml of sterile water, 50 ml of 10% Chlorhexidine and 20 ml of 2% methylcellulose gel, which is vigorously stirred with an overhead stirrer. Sodium bicarbonate of sufficient quantity and sodium borate 2.6 gm are slowly added while stirring until dissolved to provide a pH in the range of 6.0-7.0. Two gram of 2% bupivacaine is added following the addition of buffer. The viscosity of the solution is between 10,000-50,000 cps at 25° C. The solution remains stable for over 48 hours. The sample obtained is C5-MC0.4-SBI/SBO-BUP.

### Example 142a and 142b

a) To a 100 ml beaker is added 25.4 ml of sterile water, 20 ml of 2% povidone-iodine and 50 ml of 5% methylcellulose gel, which are vigorously mixed with an overhead stirrer. Sodium bicarbonate of sufficient quantity and sodium borate 2.6 gm are slowly added while stirring until dissolved to provide a pH in the range of 6.0-7.0. Two gram of 2% Lidocaine is added following the addition of buffer. The solution remains stable for over 48 hours. The sample obtained is PI0.4-MC2.5-SBI/SBO-L.
b) To a 100 ml beaker is added 25.4 ml of sterile water, 20 ml of 2% povidone-iodine and 50 ml of 5% methylcellulose gel, which are vigorously mixed with an overhead stirrer. Sodium bicarbonate of sufficient quantity and sodium borate 2.6 gm are slowly added while stirring until dissolved to provide a pH in the range of 6.0-7.0. Two gram of 2% polymyxin B sulfate is added following the addition of buffer. The solution remains stable for over 48 hours. The sample obtained is PI0.4-MC2.5-SBI/SBO-POLY-B.

### Example 143a and 143b

a) To a 100 mlmL beaker is added 15.4 ml of sterile water, 60 ml of 4% povidone-iodine and 20 ml of 2% methylcellulose gel, which are vigorously mixed with an overhead stirrer. Sodium bicarbonate of sufficient quantity and sodium borate 2.6 gm are slowly added while stirring until dissolved to provide a pH in the range of 6.0-7.0. Two gram of 2% Lidocaine is added following the addition of buffer. The solution remains stable for over 48 hours. The sample obtained is PI2.4-MC0.4-SBI/SBO-L.
b) To a 100 mlmL beaker is added 15.4 ml of sterile water, 60 ml of 4% povidone-iodine and 20 ml of 2% methylcellulose gel, which are vigorously mixed with an overhead stirrer. Sodium bicarbonate of sufficient quantity and sodium borate 2.6 gm are slowly added while stirring until dissolved to provide a pH in the range of 6.0-7.0. Two grams of 2% neomycin sulfate is added following the addition of buffer. The solution remains stable for over 48 hours. The sample obtained is PI2.4-MC0.4-SBI/SBO-NS.

### Example 144a and 144b

a) To a 100 mL beaker is added 25.4 ml of sterile water, 60 ml of 4% povidone-iodine and 10 ml of 1% methylcellulose gel, which are vigorously mixed with an overhead stirrer. Sodium bicarbonate of sufficient quantity and sodium borate 2.6 gm are slowly added while stirring until dissolved to provide a pH in the range of 6.0-7.0. Two gram of 2% Lidocaine is added following the addition of buffer. The solution remains stable for over 48 hours. The sample obtained is PI2.4-MC0.1-SBI/SBO-L.
b) To a 100 ml beaker is added 25.4 ml of sterile water, 60 ml of 4% povidone-iodine and 10 ml of 1% methylcellulose gel, which are vigorously mixed with an overhead stirrer. Sodium bicarbonate of sufficient quantity and sodium borate 2.6 gm are slowly added while stirring until dissolved to provide a pH in the range of 6.0-7.0. Two gram of 2% gramicidin is added following the addition of buffer. The solution remains stable for over 48 hours. The sample obtained is PI2.4-MC0.1-SBI/SBO-GRA.

### Example 145a and 145b

a) To a 100 ml beaker is added 5.4 ml of sterile water, 50 ml of 5% povidone-iodine and 40 ml of 4% methylcellulose gel which are vigorously mixed with an overhead stirrer. Sodium bicarbonate of sufficient quantity and sodium borate 2.6 gm are slowly added while stirring until dissolved to provide a pH in the range of 6.0-7.0. Two gram of 2% Lidocaine is added following the addition of buffer. The solution remains stable for over 48 hours. The sample obtained is PI2.5-MC1.6-SBI/SBO-L.
b) To a 100 ml beaker is added 5.4 ml of sterile water, 50 ml of 5% povidone-iodine and 40 ml of 4% methylcellulose gel which are vigorously mixed with an overhead stirrer. Sodium bicarbonate of sufficient quantity and sodium borate 2.6 gm are slowly added while stirring until dissolved to provide a pH in the range of 6.0-7.0. Two gram of 2% zinc bacitracin is added following the addition of buffer. The solution remains stable for over 48 hours. The sample obtained is PI2.5-MC1.6-SBI/SBO-ZB.

### Example 146a and 146b

a) To a 100 ml beaker is added 5.4 ml of sterile water, 60 ml of 6% povidone-iodine and 30 ml of 3% methylcellulose gel which are vigorously mixed with an overhead stirrer. Sodium bicarbonate of sufficient quantity and sodium borate 2.6 gm are slowly added while stirring until dissolved to provide a pH in the range of 6.0-7.0. Two gram of 2% Lidocaine is added following the addition of buffer. The solution remains stable for over 48 hours. The sample obtained is PI3.6-MC0.9-SBI/SBO-L.
b) To a 100 ml beaker is added 5.4 ml of sterile water, 60 ml of 6% povidone-iodine and 30 ml of 3% methylcellulose gel which are vigorously mixed with an overhead stirrer. Sodium bicarbonate of sufficient quantity and sodium borate 2.6 gm are slowly added while stirring until dissolved to provide a pH in the range of 6.0-7.0. Two gram of 2% gentamicin is added following the addition of buffer. The solution remains stable for over 48 hours. The sample obtained is PI3.6-MC0.9-SBI/SBO-GEN.

### Example 147a and 147b

a) To a 100 ml beaker is added 44.4 ml of sterile water, 1 ml of 1% benzalkonium chloride and 50 ml of 5% methylcellulose gel which are vigorously mixed with an overhead stirrer. Sodium bicarbonate of sufficient quantity and sodium borate 2.6 gm are slowly added while stirring until dissolved to provide a pH in the range of 6.0-7.0. Two gram of 2% Lidocaine is added following the addition of buffer. The solution remains stable for over 48 hours. The sample obtained is BK0.01-MC2.5-SBI/SBO-L.
b) To a 100 ml beaker is added 45.2 ml of sterile water, 0.2 ml of 2% benzalkonium chloride and 50 ml of 5% methylcellulose gel which are vigorously mixed with an overhead stirrer. Sodium bicarbonate of sufficient quantity and sodium borate 2.6 gm are slowly added while stirring until dissolved to provide a pH in the range of 6.0-7.0. Two gram of 2% chloramphenicol is added following the addition of buffer. The solution remains stable for over 48 hours. The sample obtained is BK0.004-MC2.5-SBI/SBO-CHL.

### Example 148a and 148b

a) To a 100 ml beaker is added 44.7 ml of sterile water, 0.7 ml of 1% benzalkonium chloride and 50 ml of 5% methylcellulose gel which are vigorously mixed with an overhead stirrer. Sodium bicarbonate of sufficient quantity and sodium borate 2.6 gm are slowly added while stirring until dissolved to provide a pH in the range of 6.0-7.0. Two gram of 2% Lidocaine is added following the addition of buffer. The solution remains stable for over 48 hours. The sample obtained is BK0.007-MC2.5-SBI/SBO-L.
b) To a 100 ml beaker is added 44.4 ml of sterile water, 1 ml of 1% benzalkonium chloride and 50 ml of 5% methylcellulose gel which are vigorously mixed with an overhead stirrer. Sodium bicarbonate of sufficient quantity and sodium borate 2.6 gm are slowly added while stirring until dissolved to provide a pH in the range of 6.0-7.0. Two gram of 2% tobramycin is added following the addition of buffer. The solution remains stable for over 48 hours. The sample obtained is BK0.01-MC2.5-SBI/SBO-TOB.

### Example 149a and 149b

a) To a 100 mL beaker is added 84.4 ml of sterile water, 1 ml of 1% benzalkonium chloride and 10 ml of 1% methylcellulose gel which are vigorously mixed with an overhead stirrer. Sodium bicarbonate of sufficient quantity and sodium borate 2.6 gm are slowly added while stirring until dissolved to provide a pH in the range of 6.0-7.0. Two gram of 2% Lidocaine is added following the addition of buffer. The solution remains stable for over 48 hours. The sample obtained is BK0.01-MC0.1-SBI/SBO-L.
b) To a 100 ml beaker is added 85.1 ml of sterile water, 0.3 ml of 1% benzalkonium chloride and 10 ml of 1% methylcellulose gel which are vigorously mixed with an overhead stirrer. Sodium bicarbonate of sufficient quantity and sodium borate 2.6 gm are slowly added while stirring until dissolved to provide a pH in the range of 6.0-7.0. Two gram of 2% erythromycin is added following the addition of buffer. The solution remains stable for over 48 hours. The sample obtained is BK0.003-MC0.1-SBI/SBO-ERY.

### Example 150a and 150b

a) To a 100 ml beaker is added 54.4 ml of sterile water, 1 ml of 1% benzalkonium chloride and 30 ml of 3% methylcellulose gel which are vigorously mixed with an overhead stirrer. Sodium bicarbonate of sufficient quantity and sodium borate 2.6 gm are slowly added while stirring until dissolved to provide a pH in the range of 6.0-7.0. Two gram of 2% Lidocaine is added following the addition of buffer. The solution remains stable for over 48 hours. The sample obtained is BK0.01-MC0.9-SBI/SBO-L.
b) To a 100 ml beaker is added 65.3 ml of sterile water, 0.1 ml of 5% benzalkonium chloride and 30 ml of 3% methylcellulose gel which are vigorously mixed with an overhead stirrer. Sodium bicarbonate of sufficient quantity and sodium borate 2.6 gm are slowly added while stirring until dissolved to provide a pH in the range of 6.0-7.0. Two gram of 2% tetracycline HCl is added following the addition of buffer. The solution remains stable for over 48 hours. The sample obtained is BK0.005-MC0.9-SBI/SBO-TET.

### Example 151

To a 100 ml beaker is added 44.4 ml of sterile water 1 ml of 1% benzalkonium and 50 ml of 5% methylcellulose gel which are vigorously mixed with an overhead stirrer. Sodium bicarbonate of sufficient quantity and sodium borate 2.6 gm are slowly added while stirring until dissolved to provide a pH in the range of 6.0-7.0. Two gram of 2% Lidocaine is added following the addition of buffer. The solution remains stable for over 48 hours. The sample obtained is BK0.01-MC2.5-SBI/SBO-L.

### Example 152

To a 100 mlmL beaker is added 25.4 ml of sterile water, 20 ml of 2% chlorobutanol and 50 ml of 5% methylcellulose gel which are vigorously mixed with an overhead stirrer. Sodium bicarbonate of sufficient quantity and sodium borate 2.6 gm are slowly added while stirring until dissolved to provide a pH in the range of 6.0-7.0. Two gram of 2% Lidocaine is added following the addition of buffer. The solution remains stable for over 48 hours. The sample obtained is CB0.4-MC2.5-SBI/SBO-L.

### Example 153

To a 100 mL beaker is added 77.4 ml of sterile water, 8 ml of 4% chlorobutanol and 10 ml of 1% methylcellulose gel which are vigorously mixed with an overhead stirrer. Sodium bicarbonate of sufficient quantity and sodium borate 2.6 gm are slowly added while stirring until dissolved to provide a pH in the range of 6.0-7.0. Two gram of 2% Lidocaine is added following the addition of buffer. The solution remains stable for over 48 hours. The sample obtained is CB0.32-MC0.1-SBI/SBO-L.

### Example 154

To a 100 mL beaker is added 25.4 ml of sterile water, 20 ml of 1% chlorobutanol and 50 ml of 5% methylcellulose gel which are vigorously mixed with an overhead stirrer. Sodium bicarbonate of sufficient quantity and sodium borate 2.6 gm are slowly added while stirring until dissolved to provide a pH in the range of 6.0-7.0. Two gram of 2% Lidocaine is added following the addition of buffer. The solution remains stable for over 48 hours. The sample obtained is CB0.2-MC2.510-SBI/SBO-L.

### Example 155

To a 100 ml beaker is added 45.4 ml of sterile water, 20 ml of 2% chlorobutanol and 30 ml of 3% methylcellulose gel which are vigorously mixed with an overhead stirrer. Sodium bicarbonate of sufficient quantity and sodium borate 2.6 gm are slowly added while stirring until dissolved to provide a pH in the range of 6.0-7.0. Two gram of 2% Lidocaine is added following the addition of buffer. The solution remains stable for over 48 hours. The sample obtained is CB0.4-MC0.9-SBI/SBO-L.

### Example 156

To a 100 ml beaker is added 55.4 ml of sterile water, 20 ml of 2% chlorobutanol and 20 ml of 2% methylcellulose gel which are vigorously mixed with an overhead stirrer. Sodium bicarbonate of sufficient quantity and sodium borate 2.6 gm are slowly added while stirring until dissolved to provide a pH in the range of 6.0-7.0. Two gram of 2% Lidocaine is added following the addition of buffer. The solution remains stable for over 48 hours. The sample obtained is CB0.4-MC0.4-SBI/SBO-L.

The abbreviations above have the following meanings:
lidocaine=L,
tetracaine=T,
narcaine=N,
mepivicaine=M,
proparacaine=PRO,
bupivacaine=BUP,
polymyxin B sulfate=POLY-B,
neomycin sulfate=NS,
gramicidin=GRA,
zinc bacitracin=ZB,
gentamicin=GEN,
chloramphenicol=CHL,
tobramycin=TOB
erythromycin=ERY, and
tetracycline HCl=TET.

### Antimicrobial Assays

The buffered solutions of this invention are tested for antimicrobial activity against 3 key bacteria causing endophthalmitis (Staphylococcus aureus ATCC 29213; Staphylococcus epidermidis ATTC 12228; and Streptococcus oralis ATCC) using the following protocol.

EXPERIMENT 1: In vitro experiments are performed using the following sample solutions:
1) a negative control comprising a sodium chloride solution with pH 4,
2) A solution of the antiseptic without buffer at a pH<6,
3) A solution of the antiseptic buffered to pH between 6-7,
4) A solution of the antiseptic+gel without buffer at a pH<6, s
5) A solution of the antiseptic+gel buffered to pH between 6-7.

Three, well characterized bacteria (Staphylococcus aureus ATCC 29213; Staphylococcus epidermidis ATTC 12228; and Streptococcus oralis ATCC 9811) are used to assess killing by the sample solutions above using the standardized antimicrobial drug susceptibility testing protocols described in Clinical Laboratory Standards Institute (CLSI): M02-A12, “Performance Standards for Antimicrobial Disk Susceptibility Tests; Approved Standard-12 edition, 2015. CLSI M02-A12 delineates the standardized inoculum and bacterial lawn preparation, specifying the use of Mueller Hinton agar plates, the incubation temperature and length of incubation.

Following the CLSI protocol above, a standardized suspension of each organism is made and a lawn of the prepared inoculum for each bacteria is made on to standardized drug susceptibility testing media. In lieu of the antimicrobial disks mentioned in the CLSI protocol, each sample solution is spotted/dropped (50 microliters) onto the lawn prepared for each organism. Plates are incubated for 24 hours and examined for any zone of inhibition. All organisms will show no inhibition by the negative control (sodium chloride solution at pH 4). All 4 sample solutions should show equivalent inhibition for each bacteria tested.

EXPERIMENT 2—A time-kill experiment is performed to assess the effectiveness of the sample solutions of this invention in killing of Streptococcus oralis (an organism with a thick cell wall). In this experiment, S. oralis (1×10⁶ cfu/ml) is exposed to a sample solution for 15 seconds. An equal to or greater than a 5 log drop in viability is achieved with this 15 second exposure to the sample solutions of this invention.

EXPERIMENT 3: An experiment for assessing the stability of the sample solutions is performed by testing for antimicrobial activity against 4 key bacteria causing endophthalmitis after refrigerated storage. Four well characterized bacteria (Pseudomonas aeruginosa 27853; Staphylococcus aureus 29213; Staphylococcus epidermidis 12228; and Klebsiella pneumoniae 13883) are used to assess killing by the sample solutions of this invention. The standardized CLSI antimicrobial drug susceptibility testing protocols discussed above are used. A standardized suspension of each organism is made and a lawn of the prepared inoculum for each bacteria (again using a standardized method) is made on to standardized drug susceptibility testing media. Each sample solution is then spotted/dropped (0.01 milliliters) onto the lawn prepared for each organism. Plates are incubated for 24 hours and examined for any zone of inhibition. The sample solutions should show complete inhibition of bacteria.

This same experiment is performed using the same sample solutions after refrigeration at 4° C. after 6 and 14 days, respectively. The sample solutions of this invention should show complete inhibition of bacteria after refrigerated storage for 6 days and 14 days.

Sample solutions of this invention are effective in killing on contact: Pseudomonas aeruginosa; Staphylococcus aureus; Staphylococcus epidermidis; Streptococcus oralis and Klebsiella pneumoniae.

### Application to the Skin

The buffered antiseptic solutions of this invention can be applied directly to a laceration, burn or abrasion or other cutaneous wound in the skin. Preferably they are applied with the use of a swab saturated with the buffered antiseptic solutions. The buffered antiseptic solutions will reduce the stinging sensation once applied as compared to an unbuffered antiseptic solution. The use of a gel will reduce run-off of the buffered antiseptic solution.

### Application to the Gums

The buffered antiseptic solutions of this invention can be applied directly to a cut or wound in the gums. The solutions can be applied by syringe or dropper or with a small swab saturated with the buffered antiseptic solution. The buffered antiseptic solutions will reduce the stinging sensation once applied to the cut or wound in the gums as compared to an unbuffered antiseptic solution. The use of a gel will reduce will enhance adherence to the cut or wound in the gums.

### Application to the Eye

The buffered antiseptic solutions of this invention can be applied directly to the eye, preferably with a dropper or disposable vials of small volume (about 0.3 ml). The use of a gel reduces run-off of the buffered antiseptic solution and enhances the exposure of the eye to the antiseptic. A typical procedure is to apply 2-3 drops of the buffered antiseptic solution and have the patient blink so as to coat the eyelashes. Where the procedure is to precede an intravitreal injection (IVI), a speculum may be applied once the eyelashes are coated.

### Human Eye Irritation Studies

A volunteer with healthy eyes and no signs of disease applies a buffered antiseptic solution of this invention to one eye and the same antiseptic solution, unbuffered, to the other eye. The volunteer will experience irritation vision loss in the eye with the unbuffered antiseptic solution for a much longer period (possibly hours) than the eye which received the buffered antiseptic solution.

The following experiments illustrate the improvements in patient comfort and levels of antiseptic retained on treated surfaces obtained with the compositions of the present invention.

Six subjects for a total of 12 eyes were tested. Each subject received a standard 5% Betadine solution in a randomly selected eye and the other eye received 1 of two buffered betadine solutions (pH 6.8) with added (Poloxamer) gel. The Poloxamer gel was added to the two buffered betadine solutions at 5% and 10 wt. %, based on the total weight of the composition, respectively. Vision was tested using a near card prior to application and then at 30 min, 1 hr, 2 hr, and 3 hr intervals after treatment. The following scale was used to record Visual Acuity in the tables below, 0=20/20, 1=20/25, 2=20/30, 3=20/40, and 4=20/50.

### Composition: 5% Betadine

### Visual Acuity (VA)

| Subject | Eye | Init | ½ Hr | 1 Hr | 2 Hr | 3 Hr |
|---|---|---|---|---|---|---|
| 1 | Right | 0 | 2 | 3 | 1 | 1 |
| 2 | Left | 0 | 0 | 0 | 1 | 0 |
| 3 | Left | 0 | 1 | 2 | 2 | 1 |
| 4 | Left | 0 | 2 | 2 | 1 | 2 |
| 5 | Left | 0 | 0 | 0 | 1 | 0 |
| 6 | Right | 0 | 1 | 2 | 3 | 3 |

### Composition: 5% Betadine/5% Poloxamer Gel

### Visual Acuity (VA)

| Subject | Eye | Init | ½ Hr | 1 Hr | 2 Hr | 3 Hr |
|---|---|---|---|---|---|---|
| 1 | Left | 1 | 1 | 1 | 1 | 1 |
| 2 | Right | 0 | 0 | 1 | 2 | 1 |
| 3 | Right | 0 | 0 | 0 | 0 | 0 |

### Composition: 5% Betadine/10% Poloxamer Gel

### Visual Acuity (VA)

| Subject | Eye | Init | ½ Hr | 1 Hr | 2 Hr | 3 Hr |
|---|---|---|---|---|---|---|
| 4 | Right | 0 | 1 | 1 | 1 | 1 |
| 5 | Right | 0 | 0 | 0 | 0 | 0 |
| 6 | Left | 0 | 1 | 0 | 0 | 0 |

Every subject experienced more vision loss with 5% betadine compared to the buffered solutions containing gel of the present invention. The use of higher levels of gel (10% by weight) resulted little or no loss after application (0=20/20, 1=20/25).

Pain was also subjectively rated by the subjects at the same time on a scale of 0 to 10, with no pain =0 and extreme eye pain =10. Pain was rated on initial application then at 30 min, 1 hr, 2 hr, and 3 hr intervals after treatment.

### Composition 5% Betadine

Pain

| Subject | Eye | Init | ½ Hr | 1 Hr | 2 Hr | 3 Hr |
|---|---|---|---|---|---|---|
| 1 | Right | 4 | 2 | 2 | 2 | 1 |
| 2 | Left | 3 | 5 | 5 | 3 | 1 |
| 3 | Left | 2 | 3 | 3 | 4 | 3 |
| 4 | Left | 4 | 6 | 6 | 4 | 4 |
| 5 | Left | 2 | 4 | 3 | 2 | 2 |
| 6 | Right | 3 | 4 | 5 | 5 | 4 |

### Composition 5% Betadine/5% Poloxamer Gel

Pain

| Subject | Eye | Init | ½ Hr | 1 Hr | 2 Hr | 3 Hr |
|---|---|---|---|---|---|---|
| 1 | Left | 3 | 1 | 0 | 0 | 0 |
| 2 | Right | 2 | 2 | 2 | 1 | 1 |
| 3 | Right | 1 | 1 | 0 | 0 | 0 |

### Composition 5% Betadine/10% Poloxamer Gel

Pain

| Subject | Eye | Init | ½ Hr | 1 Hr | 2 Hr | 3 Hr |
|---|---|---|---|---|---|---|
| 4 | Right | 3 | 0 | 0 | 0 | 0 |
| 5 | Right | 2 | 1 | 1 | 0 | 0 |
| 6 | Left | 2 | 0 | 0 | 0 | 0 |

Every subject experienced more pain with 5% betadine compared to the buffered solutions containing gel of the present invention.

The following experiments illustrate the improvements in adherence to anatomic surfaces obtained with the compositions of the present invention that enable extended contact time with these surfaces.

A standard 5% Betadine solution as used in the tests for patient comfort above was compared to two buffered betadine solutions of the present invention. One buffered betadine solution (pH 6.8) contained 5 wt. % Poloxamer gel based on the total weight of the composition and the other buffered betadine solution (pH 6.8) contained 10 wt. % of Poloxamer gel based on the weight of the composition. Adherence to false eyelashes was determined by measuring the weight of the false eyelash before and after submersion into the solutions. The eyelashes were allowed to “drip dry” for 30 seconds prior to re-weighing them. Each of the 3 solutions were tested 3 times. Each test was performed with new eyelashes.

| Eyelash Adherence (mg) | Test 1 | Test 2 | Test 3 | Average |
|---|---|---|---|---|
| 5% Betadine | 38 | 23 | 32 | 31 |
| 5% Betadine/5% Poloxamer gel | 58 | 82 | 70 | 70 |
| 5% Betadine/ 10% Poloxamer gel | 72 | 91 | 101 | 88 |

The results above show that the milligrams of adherent solution were lowest with 5% Betadine and highest with 5% Betadine/10% Poloxamer. The 5% Betadine/5% Poloxamer was in the middle.

1. An ophthalmic formulation comprising povidone-iodine in an amount of 1.0% to 20.0% by weight, based on the total weight of the ophthalmic formulation, and an amount of buffering agent sufficient to provide a pH in the range of 6.5-7.5 for said ophthalmic formulation, which additionally comprises an aqueous gel or semi-gel formulation in an amount greater than 5% by weight based on the total weight of the ophthalmic formulation, wherein said ophthalmic formulation does not contain lidocaine.

2. An ophthalmic formulation of claim 1 wherein the aqueous gel or semi-gel formulation is within a concentration range of greater than 5% to 15% by weight based on the total weight of the ophthalmic formulation.

3. An ophthalmic formulation of claim 1, wherein Lidocaine and other anesthetic agents are excluded, anesthetic agents.

4. An ophthalmic formulation of claim 1 which is shelf stable for over 6 months at ambient room temperature.

5. An ophthalmic formulation of claim 1 having a pH in the range of 7.0 to 7.4.

6. An ophthalmic formulation of claim 1 which comprises 1% to 10% by weight of povidone-iodine.

7. An ophthalmic formulation of claim 1 wherein the aqueous gel or semi-gel formulation is present in an amount sufficient to provide a viscosity for said ophthalmic formulation in the range of 10,000 cps to 50,000 cps at about 25° C.

8. An ophthalmic formulation of claim 1, wherein said buffering agent is boric acid, sodium borate, sodium hydroxide, sodium chloride, sodium acetate, sodium carbonate, sodium bicarbonate, sodium bisulfite, trisodium citrate, potassium tetroxalate dihydrate (KH3(C2O4)2.2H2O), phosphate buffered saline (PBS) or borate buffered saline (BBS).

9. An ophthalmic formulation of claim 1, wherein said aqueous gel or semi-gel formulation is selected from the group consisting of hydroxypropyl cellulose, methyl hydroxypropyl cellulose, hydroxypropyl methyl cellulose, cellulose acetate, methyl cellulose, ethyl cellulose, carboxymethyl cellulose salt, carboxymethyl cellulose sodium, methyl hydroxyethyl cellulose, hydroxyethyl cellulose, cellulose gum, dextran, polyvinyl alcohol, polyvinyl acrylates, polymeric mixtures of polyvinyl acrylates, aqueous cross-linked acrylic polymers, poly acrylic acid, pluronic polyol polymers, poloxamer, polyols, carboxy vinyl polymers and carbomers.

10. An ophthalmic formulation of claim 1, wherein said aqueous gel or semi-gel formulation is Poloxamer.

11. An ophthalmic formulation of claim 1, further comprising a steroid, a preservative or a combination thereof.

12. An ophthalmic formulation of claim 1, which is isotonic for treatment of an eye.

13. A method for antisepsis of an ocular surface comprising:
providing an ophthalmic formulation comprising povidone-iodine in an amount of 1.0% to 20.0% by weight, based on the total weight of the ophthalmic formulation, an amount of buffering agent sufficient to provide a pH in the range of 6.5-7.5 for said ophthalmic formulation, and an aqueous gel or semi-gel formulation in an amount greater than 5% by weight based on the total weight of the ophthalmic formulation; and
contacting said ocular surface with said ophthalmic formulation for a period of time sufficient to achieve antiseptic treatment of said ocular surface.

14. The method of claim 13, wherein said ocular surface comprises a cornea, conjunctiva, eyelids and eyelashes.

15. The method of claim 14, wherein said ocular surface comprises a cornea.

16. The method of claim 13 which provides sufficient antisepsis of an ocular surface for an intravitreal injection (IVI), intraocular surgery, and/or anterior chamber paracentesis.

17. A method which comprises applying a composition of claim 1 to an eye. performing an ophthalmic procedure on said eye and removing the composition of claim 1 by irrigation.
